(19) United States
(12) Patent Application Publication
(10) Pub. No.: US 2024/0174995 A1
(43) Pub. Date: May 30, 2024

(54) SYSTEM AND METHOD FOR GENOME EDITING BASED ON C2C1 NUCLEASES

(71) Applicant: Institute of Zoology, Chinese Academy of Sciences, Beijing (CN)

(72) Inventors: Wei Li, Beijing (CN); Qi Zhou, Beijing (CN); Fei Teng, Beijing (CN)

(73) Assignee: Institute of Zoology, Chinese Academy of Sciences, Beijing (CN)

(21) Appl. No.: 17/290,806

(22) PCT Filed: Nov. 30, 2018
(86) PCT No.: PCT/CN2018/118458

§ 371 Date: May 3, 2021

(30) Foreign Application Priority Data
Nov. 2, 2018 (CN) 201811300251.6

(51) Int. Cl. C12N 9/22; A61K 38/00; A61K 48/00; C12N 15/113

(52) U.S. Cl. CPC C12N 9/22; A61K 48/005; C12N 15/113; A61K 38/00

(57) ABSTRACT

The invention relates to the field of genetic engineering. In particular, the present invention relates to a genome editing system and method based on C2c1 nuclease. The present invention also relates to artificial guide RNA which can be combined with the C2c1 nuclease and used for genomic editing

### CROSS REFERENCE TO RELATED APPLICATIONS

This application is a U.S. National Phase Application of PCT International Patent Application PCT/CN2018/118458, filed Nov. 30, 2018, which claims the benefit of Chinese Patent Application Serial No. 201811300251.6, filed Nov. 2, 2018, the disclosure of each of which is incorporated herein by reference in its entirety.

### REFERENCE TO SEQUENCE LISTING

The Sequence Listing associated with the instant disclosure has been electronically submitted to the United States Patent and Trademark Office as a 177 kilobyte ASCII text file created on Jul. 27, 2021 and entitled “1547_35_PCT_US_ST25.txt”. The Sequence Listing submitted via EFS-Web is hereby incorporated by reference in its entirety.

### TECHNICAL FIELD

The invention relates to the field of genetic engineering. In particular, the present invention relates to genome editing systems based on C2c1 nuclease and methods. The present invention also relates to artificial guide RNA which can be combined with C2c1 nuclease and used for genome editing

### BACKGROUND

Precise genome editing is a promising area for its bright prospects in gene therapy, especially with the appearance of CRISPR-Cas (Clustered Regularly Interspaced Short Palindromic Repeats-CRISPR-associated proteins) systems. Up to now, three types of CRISPR-Cas systems, type II Cas9(Cong, L. et al. Science 339, 819-823 (2013); Mali, P. et al. Science 339, 823-826 (2013)), type V-A Cpf13 (Zetsche, B. et al. Cell 163, 759-771 (2015)) and type V-B C2c1, have been successfully harnessed to facilitate mammalian genome engineering. For type II and V CRISPR-Cas systems, the guide RNA and Cas effector protein are the two core components for target DNA recognition and cleavage (Wright, A. V., Nunez, J. K. & Doudna, J. A. Cell 164, 29-44 (2016); Shmakov, S. et al. Nat Rev Microbiol 15, 169-182 (2017)). Previous studies indicated that the RNA and protein components were interchangeable in closely related Cas9 systems (Fonfara, I. et al. Nucleic Acids Res 42, 2577-2590 (2014)) as well as Cpf1 systems (Zetsche, B. et al. Cell 163, 759-771 (2015)), and could be preliminarily optimized (Nishimasu, H. et al. Cell 156, 935-949 (2014); Zalatan, J. G. et al. Cell 160, 339-350 (2015)). Though a number of emerging CRISPR-Cas systems and boosting investigations promote the wide applications of CRISPR-Cas systems (Wright, A. V., Nunez, J. K. & Doudna, J. A. Cell 164, 29-44 (2016); Shmakov, S. et al. Nat Rev Microbiol 15, 169-182 (2017)), there is still little known about how to re-design or even de novo synthesize an enzymatic genome-engineering system.

The Type V-B CRISPR-C2c1 system is an emerging promising technology for genome engineering. However, C2c1, which can be used for mammalian genome editing, is rare, greatly limiting its application. There remains a need in the art for new C2c1 nuclease-based genome editing systems that can be used for mammalian genome editing.

### SUMMARY

In one aspect, the present invention provides a genome editing system for site-directed modification of a target sequence in the genome of a cell, comprising at least one of the following i) to v):

i) a C2c1 protein or variant thereof, and a guide RNA;

ii) an expression construct comprising a nucleotide sequence encoding a C2c1 protein or variant thereof, and a guide RNA;

iii) a C2c1 protein or variant thereof, and an expression construct comprising a nucleotide sequence encoding a guide RNA;

iv) an expression construct comprising a nucleotide sequence encoding a C2c1 protein or variant thereof, and an expression construct comprising a nucleotide sequence encoding a guide RNA;

v) an expression construct comprising a nucleotide sequence encoding a C2c1 protein or variant thereof and a nucleotide sequence encoding a guide RNA;

wherein the guide RNA is capable of forming complex with the C2c1 protein or variant thereof and targeting the C2c1 protein ortholog or variant thereof to the target sequence in the genome of the cell.

In some embodiments, the C2c1 protein is AaC2c1 protein derived from Alicyclobacillus acidiphilus, AkC2c1 protein derived from Alicyclobacillus kakegawensis, AmC2c1 protein derived from Alicyclobacillus macrosporangiidus, BhC2c1 protein derived from Bacillus hisashii, BsC2c1 protein derived from the genus Bacillus, Bs3C2c1 protein derived from the genus Bacillus, DiC2c1 protein derived from Desulfovibrio inopinatus, LsC2c1 protein derived from Laceyella sediminis, SbC2c1 protein derived from Spirochaetes bacterium, TcC2c1 protein derived from Tuberibacillus calidus. For example, the C2c1 protein is the AaC2c1 protein derived from Alicyclobacillus acidiphilus NBRC 100859, the AkC2c1 protein derived from Alicyclobacillus kakegawensis NBRC 103104, the AmC2c1 protein derived from Alicyclobacillus macrosporangiidus strain DSM 17980, the BhC2c1 protein derived from Bacillus hisashii strain C4, the BsC2c1 protein derived from genus Bacillus NSP2.1, the Bs3C2c1 protein derived from genus Bacillus V3-13 contig_40, the DiC2c1 protein derived from Desulfovibrio inopinatus DSM 10711, the LsC2c1 protein derived from Laceyella sediminis strain RHA1, the SbC2c1 protein derived from Spirochaetes bacterium GWB1_27_13, the TcC2c1 protein derived from Tuberibacillus calidus DSM 17572.

In the second aspect, the present invention provides a method of site-directed modifying a target sequence in the genome of a cell, comprising introducing the genome editing system of the invention into the cell.

In the third aspect, the invention provides a method of treating a disease in a subject in need thereof, comprising delivering to the subject an effective amount of a genome editing system of the invention to modify a gene related with the disease in the subject.

In the fourth aspect, the invention provides the use of a genome editing system of the invention for the preparation of a pharmaceutical composition for treating a disease in a subject in need thereof, wherein the genome editing system is for modifying a gene related with the disease in the subject.

In a fifth aspect, the invention provides a kit for use in the method of the invention, the kit comprising the genome editing system of the invention, and instructions for use.

In the sixth aspect, the invention provides a pharmaceutical composition for treating a disease in a subject in need thereof, comprising a genome editing system of the invention and a pharmaceutically acceptable carrier, wherein the genome editing system is for modifying a gene related with the disease in the subject.

### BRIEF DESCRIPTION OF THE FIGURES

FIGS. 1A and 1B. Phylogenetic tree of non-redundant C2c1 orthologs and their loci chosen for genome-editing testing. (FIG. 1A) Neighbor Joining phylogenetic tree showing the evolutionary relationships of C2c1 orthologs tested in this study. (FIG. 1B) Maps of bacterial genomic loci corresponding to the 8 C2c1 proteins highlighted in (FIG. 1A). In silico co-folding of the crRNA DR and putative tracrRNA shows stable secondary structure. DR, direct repeat. The number of each bacterial genomic spacers is indicated above or below their CRISPR array.

FIG. 2. Protein alignment of C2c1 orthologs. Multiple sequence alignment of the amino acid sequences the 10 C2c1 orthologs tested in this study (SEQ ID NOs:1-10). Residues that are conserved are highlighted with a red background and conserved mutations are highlighted with an outline and red font.

FIGS. 3A-3C. C2c1 orthologs mediated genome targeting in human 293T cells. (FIG. 3A) T7EI assay results indicating the genome targeting activity of the eight C2c1 proteins combined with their cognate sgRNAs in the human genome. Red triangles indicate the cleaved bands. (FIG. 3B) T7EI assay results indicating the simultaneous multiplex genome targeting mediated by Bs3C2c1 combined with its cognate sgRNAs (Bs3sgRNAs) in human 293T cells. (FIG. 3C) Sanger sequencing showing representative indels induced by Bs3C2c1 combined with Bs3sgRNAs. PAM and protospacer sequences are colored in red and blue, respectively. Indels and insertions are symbolled with purple dashes and green lowercases, respectively.

FIGS. 4A-4D. Orthogonal C2c1 proteins for RNA-guided genome editing. (FIG. 4A) Graphical overview of the 10 C2c1 orthologs tested in this study. Sizes (amino acids) are indicated. (FIG. 4B) T7EI assay results indicating the genome targeting activity of the eight C2c1 orthologs directed by their cognate sgRNAs in human 293T cells. Red triangles indicate the cleaved bands. (FIGS. 4C and 4D) T7EI assay results indicating the genome targeting activity of the eight C2c1 orthologs directed by AasgRNA (FIG. 4C) and AksgRNA (FIG. 25 4D) in human 293T cells. Red triangles indicate the cleaved bands.

FIG. 5. DNA alignment of sgRNAs of C2c1. Multiple sequence alignment of the DNA sequences of the 8 sgRNAs derived from the 10 C2c1 loci tested in this study.

FIGS. 6A-6E. Interchangeability between C2c1 orthologs and their sgRNAs. T7EI assay results indicating the genome targeting activity of the eight C2c1 orthologs directed by AasgRNA (FIG. 6A), AksgRNA (FIG. 6B), AmsgRNA (FIG. 6C), Bs3sgRNA (FIG. 6D) and LssgRNA (FIG. 6E) in human 293T cells. Red triangles indicate the cleaved bands.

FIGS. 7A-7F. Artificial sgRNAs mediated multiplex genome targeting. (FIG. 7A) Maps pf bacterial genomic loci corresponding to DiC2c1 and TcC2c1. The two C2c1 loci have no CRISPR array. (FIGS. 7B and 7C) T7EI assay results indicating the genome targeting activity of AaC2c1, DiC2c1 and TcC2c1 directed by AasgRNA (FIG. 7B) and AksgRNA (FIG. 7C) in the human 293T cells. Red triangles indicate the cleaved bands. (FIG. 7D) T7EI assay results indicating the simultaneous multiplex genome targeting mediated by TcC2c1 combined with AksgRNAs in human 293T cells. (FIG. 7E) Schematic illustrating the secondary structures of artificial sgRNA scaffold 13 (artsgRNA13). (FIG. 7F) T7EI assay results indicating the simultaneous multiplex genome targeting mediated by TcC2c1 combined with artsgRNA13s in human 293T cells.

FIGS. 8A-8F. Orthogonal sgRNAs directed C2c1 for genome editing.

T7EI assay results indicating the genome targeting activity of AaC2c1, DiC2c1 and TcC2c1 directed by AasgRNA (a), AksgRNA (b), AmsgRNA (c), Bs3sgRNA (d) and LssgRNA (e) in the human 293T cells. Red triangles indicate the cleaved bands.

FIGS. 9A-9C. TcC2c1-mediated multiplex genome editing. (FIG. 9A) T7EI assay results indicating the simultaneous multiplex genome targeting mediated by TcC2c1 combined with AmsgRNAs in human 293T cells. (FIGS. 9B and 9C) Sanger sequencing shows representative indels induced by TcC2c1 combined with AksgRNAs (FIG. 9B) and AmsgRNAs (FIG. 9C). PAM and protospacer sequences are colored in red and blue, respectively. Indels and insertions are symbolled with purple dashes and green lowercases, respectively.

FIGS. 10A-10C. Artificial sgRNAs directed TcC2c1 for genome editing. (FIG. 10A) Schematic illustrating the secondary structures of the 36 artificial sgRNA (artsgRNA) scaffolds (scaffold: 1-12 and 14-37). (FIG. 10B) T7EI assay results indicating the genome targeting activity of TcC2c1 directed by artsgRNAs in the human 293T cells. Red triangles indicate the cleaved bands. (FIG. 10C) T7EI assay results indicating the simultaneous multiplex genome targeting mediated by AaC2c1 combined with artsgRNA13s in human 293T cells.

### DETAILED DESCRIPTION

### 1. Definitions

In the present invention, the scientific and technical terms used herein have the meaning as commonly understood by a person skilled in the art unless otherwise specified. Also, the protein and nucleic acid chemistry, molecular biology, cell and tissue culture, microbiology, immunology related terms, and laboratory procedures used herein are terms and routine steps that are widely used in the corresponding field. For example, standard recombinant DNA and molecular cloning techniques used in the present invention are well known to those skilled in the art and are more fully described in the following document: Sambrook, J., Fritsch, E. F. and Maniatis, T., Molecular Cloning: A Laboratory Manual; Cold Spring Harbor Laboratory Press: Cold Spring Harbor, 1989 (hereinafter referred to as “Sambrook”). In the meantime, in order to better understand the present invention, definitions and explanations of related terms are provided below.

In one aspect, the present invention provides a genome editing system for site-directed modification of a target sequence in the genome of a cell, comprising at least one of the following i) to v):

i) a C2c1 protein or variant thereof, and a guide RNA;

ii) an expression construct comprising a nucleotide sequence encoding a C2c1 protein or variant thereof, and a guide RNA;

iii) a C2c1 protein or variant thereof, and an expression construct comprising a nucleotide sequence encoding a guide RNA;

iv) an expression construct comprising a nucleotide sequence encoding a C2c1 protein or variant thereof, and an expression construct comprising a nucleotide sequence encoding a guide RNA;

v) an expression construct comprising a nucleotide sequence encoding a C2c1 protein or variant thereof and a nucleotide sequence encoding a guide RNA;

wherein the guide RNA is capable of forming complex with the C2c1 protein or variant thereof and targeting the C2c1 protein or variant thereof to the target sequence in the genome of the cell. In some embodiments, the targeting results in substitution, deletion and/or addition of one or more nucleotides in the target sequence.

“Genome” as used herein encompasses not only chromosomal DNA present in the nucleus, but also organellar DNA present in the subcellular components (e.g., mitochondria, plastids) of the cell.

“C2c1 nuclease”, “C2c1 protein” and “C2c1” are used interchangeably herein and refer to an RNA-directed nuclease comprising a C2c1 protein or a fragment thereof. C2c1 has a guide RNA-mediated DNA binding activity and DNA cleavage activity, and can target and cleave DNA target sequences to form DNA double-strand breaks (DSBs) under the guidance of a guide RNA. DSB can activate the intracellular intrinsic repair mechanism, non-homologous end joining (NHEJ) and homologous recombination (HR), to repair DNA damage in cells. During the repair process, the specific DNA sequence is subjected to site-directed editing.

In some embodiments, the C2c1 protein is an AaC2c1 protein derived from Alicyclobacillus acidiphilus, an AkC2c1 protein derived from Alicyclobacillus kakegawensis, an AmC2c1 protein derived from Alicyclobacillus macrosporangiidus, a BhC2c1 protein derived from Bacillus hisashii, a BsC2c1 protein derived from the genus Bacillus, a Bs3C2c1 protein derived from the genus Bacillus, a DiC2c1 protein derived from Desulfovibrio inopinatus, a LsC2c1 protein derived from Laceyella sediminis, a SbC2c1 protein derived from Spirochaetes bacterium, a TcC2c1 protein derived from Tuberibacillus calidus.

For example, the C2c1 protein is the AaC2c1 protein derived from Alicyclobacillus acidiphilus NBRC 100859, the AkC2c1 protein derived from Alicyclobacillus kakegawensis NBRC 103104, the AmC2c1 protein derived from Alicyclobacillus macrosporangiidus strain DSM 17980, the BhC2c1 protein derived from Bacillus hisashii strain C4, and the BsC2c1l protein derived from genus Bacillus NSP2.1, the Bs3C2c1 protein derived from Bacillus genus V3-13 contig_40, the DiC2c1 protein derived from Desulfovibrio inopinatus DSM 10711, the LsC2c1 protein derived from Laceyella sediminis strain RHA1, the SbC2c1 protein derived from Spirochaetes bacterium GWB1_27_13, the TcC2c1 protein derived from Tuberibacillus calidus DSM 17572.

In some embodiments of the invention, the C2c1 protein is a C2c1 protein whose natural locus does not have a CRISPR array. In some embodiments, the C2c1 protein whose natural locus does not have a CRISPR array is a DiC2c1 or TcC2c1 protein.

In some embodiments, the C2c1 protein comprises the amino acid sequence set forth in any one of SEQ ID NOs:1-10. For example, the AaC2c1, AkC2c1, AmC2c1, BhC2c1, BsC2c1, Bs3C2c1, DiC2c1, LsC2c1, SbC2c1, and TcC2c1 proteins comprise the amino acid sequences set forth in SEQ ID NOs: 1-10, respectively.

In some embodiments, the variant of the C2c1 protein comprises an amino acid sequence having at least 80%, at least 85%, at least 90%, at least 95%, at least 96%, at least 97%, at least 98%, at least 99% sequence identity with a wild-type C2c1 protein (e.g., wild-type AaC2c1, AkC2c1, AmC2c1, BhC2c1, BsC2c1, Bs3C2c1, DiC2c1, LsC2c1, SbC2c1, or TcC2c1 protein), respectively, and has genomic editing and/or targeting activity of a wild-type C2c1 protein (e.g., wild-type AaC2c1, AkC2c1, AmC2c1, BhC2c1, BsC2c1, Bs3C2c1, DiC2c1, LsC2c1, SbC2c1, TcC2c1 protein), respectively.

In some embodiments, the variant of the C2c1 protein comprises an amino acid sequence in which one or more amino acid residues are substituted, deleted or added relative to a wild-type C2c1 protein (e.g., wild-type AaC2c1, AkC2c1, AmC2c1, BhC2c1, BsC2c1, Bs3C2c1, DiC2c1, LsC2c1, SbC2c1, TcC2c1 protein), respectively, and has genome editing and/or targeting activity of wild-type C2c1 proteins (e.g., wild-type AaC2c1, AkC2c1, AmC2c1, BhC2c1, BsC2c1, Bs3C2c1, DiC2c1, LsC2c1, SbC2c1, TcC2c1 protein). For example, the variant of the C2c1 protein comprises an amino acid sequence in which 1, 2, 3, 4, 5, 6, 7, 8, 9, 10 amino acid residue(s) are substituted, deleted or added relative to a wild-type C2c1 protein (e.g., wild-type AaC2c1, AkC2c1, AmC2c1, BhC2c1, BsC2c1, Bs3C2c1, DiC2c1, LsC2c1, SbC2c1, TcC2c1 proteins), respectively. In some embodiments, the amino acid substitution is a conservative substitution.

“Polypeptide,” “peptide,” and “protein” are used interchangeably in the present invention to refer to a polymer of amino acid residues. The terms apply to an amino acid polymer in which one or more amino acid residues is artificial chemical analogue of corresponding naturally occurring amino acid(s), as well as to a naturally occurring amino acid polymer. The terms “polypeptide,” “peptide,” “amino acid sequence,” and “protein” may also include modified forms including, but not limited to, glycosylation, lipid ligation, sulfation, Y carboxylation of glutamic acid residues, and ADP-ribosylation.

Sequence “identity” has recognized meaning in the art, and the percentage of sequence identity between two nucleic acids or polypeptide molecules or regions can be calculated using the disclosed techniques. Sequence identity can be measured along the entire length of a polynucleotide or polypeptide or along a region of the molecule. (See, for example, Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, 30) G., Academic Press, 1987; and Sequence Analysis Primer, Gribskov, M. and Devereux, J., eds., M Stockton Press, New York, 1991). Although there are many methods for measuring the identity between two polynucleotides or polypeptides, the term “identity” is well known to the skilled person (Carrillo, H. & Lipman, D., SIAM J Applied Math 48: 1073 (1988)).

Suitable conservative amino acid substitution in peptides or proteins are known to those skilled in the art and can generally be carried out without altering the biological activity of the resulting molecule. In general, one skilled in the art recognizes that a single amino acid substitution in a non-essential region of a polypeptide does not substantially alter biological activity (See, for example, Watson et al., Molecular Biology of the Gene, 4th Edition, 1987, The Benjamin/Cummings Pub. co., p.224).

In some embodiments, the variant of the C2c1 protein encompasses a nuclease-dead C2c1 protein (dC2c1). The nuclease-dead C2c1 protein refers to a C2c1 protein that retains the RNA-mediated DNA-binding activity but does not have DNA cleavage activity. In some embodiments, the nuclease-dead C2c1 protein encompasses a C2c1 nickase that cleaves only one strand of double-stranded target DNA.

In some embodiments, the variant of the C2c1 protein is a fusion protein of dC2c1 and a deaminase. For example, dC2c1 and deaminase in the fusion protein can be linked by a linker such as a peptide linker.

As used herein, “deaminase” refers to an enzyme that catalyzes a deamination reaction. In some embodiments of the invention, the deaminase refers to a cytosine deaminase capable of accepting single-stranded DNA as a substrate and capable of catalyzing the deamination of cytidine or deoxycytidine to uracil or deoxyuracil, respectively. In some embodiments of the invention, the deaminase refers to adenine deaminase capable of accepting single-stranded DNA as a substrate and capable of catalyzing adenosine or deoxyadenosine (A) into inosine (I). Base editing in the target DNA sequence, such as C to T conversion or A to G conversion, can be achieved by using a fusion protein of a C2c1 variant and a deaminase.

In some embodiments of the present invention, the C2c1 protein or variant thereof of the present invention further comprises a nuclear localization sequence (NLS). In general, one or more NLSs in the C2c1 protein or variant thereof should be of sufficient strength to drive the C2c1 protein or variant thereof in the nucleus of a plant cell to achieve an amount accumulation of base editing function. In general, the intensity of nuclear localization activity is determined by the number, location, one or more specific NLSs used of the NLS in the C2c1 protein or variant thereof, or a combination of these factors.

In some embodiments of the present invention, the NLS of the C2c1 protein or variant thereof of the present invention may be located at the N-terminus and/or C-terminus. In some embodiments, the C2c1 protein or variant thereof comprises about 1, 2, 3, 4, 5, 6, 7, 8, 9, 10 or more NLSs. In some embodiments, the C2c1 protein or variant thereof comprises about 1, 2, 3, 4, 5, 6, 7, 8, 9, 10 or more NLS at or near the N-terminus. In some embodiments, the C2c1 protein or variant thereof comprises about 1, 2, 3, 4, 5, 6, 7, 8, 9, 10 or more NLSs at or near the C-terminus. In some embodiments, the C2c1 protein or variant thereof comprises a combination of these, such as comprises one or more NLSs at the N-terminus and one or more NLSs at the C-terminus. When there is more than one NLS, each can be selected to be independent of other NLSs. In some preferred embodiments of the present invention, the C2c1 protein or variant thereof comprises two NLSs, for example, the two NLSs are located at the N-terminus and the C-terminus, respectively.

In general, NLS consists of one or more short sequences of positively charged lysine or arginine exposed on the surface of the protein, but other types of NLS are also known. Non-limiting examples of NLS include: KKRKV (SEQ ID NO: 81), PKKKRKV (SEQ ID NO: 82), or SGGSPKKKRKV (SEQ ID NO: 83).

Furthermore, depending on the location of the DNA to be edited, the C2c1 protein or variant thereof of the present invention may also include other localization sequences, such as cytoplasmic localization sequences, chloroplast localization sequences, mitochondrial localization sequences, and the like.

In some embodiments of the invention, the target sequence is 18-35 nucleotides in length, preferably 20 nucleotides. In some embodiments of the invention, the 5′-end flanking of the target sequence is a protospacer adjacent motif (PAM) sequence selected from 5′TTTN-3′, 5′ATTN-3′, 5′GTTN-3′, 5′CTTN-3′, 5′TTC-3′, 5′TTG-340 , 5′TTA-3′, 5′TTT-3′, 5′TAN-3′, 5′TGN-3′, 5′TCN-3′ and 5′ATC-3′, wherein N is selected from A, G, C and T.

In the present invention, the target sequence to be modified may be located at any location in the genome, for example, in a functional gene such as a protein-encoding gene, or may be, for example, located in a gene expression regulatory region such as a promoter region or an enhancer region, thereby the gene functional modification or gene expression modification can be achieved. The substitution, deletion and/or addition in the target sequence of the genome can be detected by T7EI, PCR/RE or sequencing methods.

“guide RNA” and “gRNA” can be used interchangeably herein, typically composed of crRNA and tracrRNA molecules that are partially complementary to each other to form a complex, wherein the crRNA comprises a sequence that is sufficiently identical to the target sequence to hybridize to the complement of the target sequence and direct the CRISPR complex (C2c1+crRNA+tracrRNA) to sequence specifically bind to the target sequence. However, a single guide RNA (sgRNA) containing both crRNA and tracrRNA characteristics can be designed and used.

In some embodiments of the invention, the guide RNA is sgRNA. In some particular embodiments, the sgRNA is encoded by a nucleotide sequence selected from the group consisting of:

(AasgRNA; SEQ ID NO: 31)
5′-GTCTAAAGGACAGAATTTTTCAACGGGTGTGCCAATGGCCACTTTCCAGGT
GGCAAAGCCCGTTGAACTTCTCAAAAAGAACGCTCGCTCAGTGTTCTGACGTCGG
ATCACTGAGCGAGCGATCTGAGAAGTGGCAC-Nₓ-3′;
(AksgRNA1; SEQ ID NO: 32)
5′-tcgtctataGGACGGCGAGGACAACGGGAAGTGCCAATGTGCTCTTTCCAAGAG
CAAACACCCCGTTGGCTTCAAGATGACCGCTCGCTCAGCGATCTGACAACGGATC
GCTGAGCGAGCGGTCTGAGAAGTGGCAC-Nₓ-3′;
(AmsgRNA1; SEQ ID NO: 33)
5′-ggaattgccgatctaTAGGACGGCAGATTCAACGGGATGTGCCAATGCACTCTTTCC
AGGAGTGAACACCCCGTTGGCTTCAACATGATCGCCCGCTCAACGGTCCGATGTC
GGATCGTTGAGCGGGCGATCTGAGAAGTGGCAC-Nₓ-3′;
(BhsgRNA; SEQ ID NO: 34)
5′-GAGGTTCTGTCTTTTGGTCAGGACAACCGTCTAGCTATAAGTGCTGCAGGG
TGTGAGAAACTCCTATTGCTGGACGATGTCTCTTTTATTTCTTTTTTCTTGGATGTCC
AAGAAAAAAGAAATGATACGAGGCATTAGCAC-Nₓ-3′;
(BssgRNA; SEQ ID NO: 35)
5′-CCATAAGTCGACTTACATATCCGTGCGTGTGCATTATGGGCCCATCCACAGG
TCTATTCCCACGGATAATCACGACTTTCCACTAAGCTTTCGAATGTTCGAAAGCTTA
GTGGAAAGCTTCGTGGTTAGCAC-Nₓ-3′;
(Bs3sgRNA; SEQ ID NO: 36)
5′-GGTGACCTATAGGGTCAATGAATCTGTGCGTGTGCCATAAGTAATTAAAAAT
TACCCACCACAGGATTATCTTATTTCTGCTAAGTGTTTAGTTGCCTGAATACTTAGC
AGAAATAATGATGATTGGCAC-Nₓ-3′;
(LssgRNA; SEQ ID NO: 37)
5′-GGCAAAGAATACTGTGCGTGTGCTAAGGATGGAAAAAATCCATTCAACCAC
AGGATTACATTATTTATCTAATCACTTAAATCTTTAAGTGATTAGATGAATTAAATGT
GATTAGCAC-Nₓ-3′;
or
(SbsgRNA; SEQ ID NO: 38)
5′-GTCTTAGGGTATATCCCAAATTTGTCTTAGTATGTGCATTGCTTACAGCGACA
ACTAAGGTTTGTTTATCTTTTTTTTACATTGTAAGATGTTTTACATTATAAAAAGAAG
ATAATCTTATTGCAC-Nₓ-3′;

wherein Nx represents nucleotide sequence that consists of X consecutive nucleotides (spacer sequence), N is independently selected from A, G, C and T; X is an integer of 18 [symbol] X [symbol] 35, preferably, X=20. In some embodiments, the sequence Nx (spacer sequence) is capable of specifically hybridizing to the complement of the target sequence. The sequence other than Nx in the sgRNA is a scaffold sequence of sgRNA. In some embodiments, the sgRNA comprises a scaffold sequence encoded by the nucleotide sequence of any one of SEQ ID NOs: 31-38.

The present inventors have surprisingly found that the C2c1 protein and the guide RNA in different C2c1 systems can be used interchangeably, thereby enabling the artificial design of universal guide RNAs.

Thus, in some embodiments, the invention provides an artificial sgRNA, which selected from the nucleotide sequence encoding:

(artsgRNA1; SEQ ID NO: 39)
5′-GGTCTAAAGGACAGAATTTTTCAACGGGTGTGCCAATGGCCACTTTCCAGG
TGGCAAAGCCCGTTGAACTTCAAGCGAAGTGGCAC-Nₓ-3′;
(artsgRNA2; SEQ ID NO: 40)
5′-GGTCTAAAGGACAGAAGACAACGGGAAGTGCCAATGTGCTCTTTCCAAGA
GCAAACACCCCGTTGACTTCAAGCGAAGTGGCAC-Nₓ-3′;
(artsgRNA3; SEQ ID NO: 41)
5′-GGTCTAAAGGACAGAAAATCTGTGCGTGTGCCATAAGTAATTAAAAATTAC
CCACCACAGACTTCAAGCGAAGTGGCAC-Nₓ-3′;
(artsgRNA4; SEQ ID NO: 42)
5′-GGTCGTCTATAGGACGGCGAGTTTTTCAACGGGTGTGCCAATGGCCACTTT
CCAGGTGGCAAAGCCCGTTGAACTTCAAGCGAAGTGGCAC-Nₓ-3′;
(artsgRNA5; SEQ ID NO: 43)
5′-GGTCGTCTATAGGACGGCGAGGACAACGGGAAGTGCCAATGTGCTCTTTCC
AAGAGCAAACACCCCGTTGACTTCAAGCGAAGTGGCAC-Nₓ-3′;
(artsgRNA6; SEQ ID NO: 44)
5′-GGTCGTCTATAGGACGGCGAGAATCTGTGCGTGTGCCATAAGTAATTAAAA
ATTACCCACCACAGACTTCAAGCGAAGTGGCAC-Nₓ-3′;
(artsgRNA7; SEQ ID NO: 45)
5′-GGTGACCTATAGGGTCAATGTTTTTCAACGGGTGTGCCAATGGCCACTTTCC
AGGTGGCAAAGCCCGTTGAACTTCAAGCGAAGTGGCAC-Nₓ-3′;
(artsgRNA8; SEQ ID NO: 46)
5′-GGTGACCTATAGGGTCAATGGACAACGGGAAGTGCCAATGTGCTCTTTCCA
AGAGCAAACACCCCGTTGACTTCAAGCGAAGTGGCAC-Nₓ-3′;
(artsgRNA9; SEQ ID NO: 47)
5′-GGTGACCTATAGGGTCAATGAATCTGTGCGTGTGCCATAAGTAATTAAAAAT
TACCCACCACAGACTTCAAGCGAAGTGGCAC-Nₓ-3′;
(artsgRNA10; SEQ ID NO: 48)
5′-GGTCTAAAGGACAGAATTTTTCAACGGGTGTGCCAATGGCCACTTTCCAGG
TGGCAAAGCCCGTTGAGCTTCAAAGAAGTGGCAC-Nₓ-3′;
(artsgRNA11; SEQ ID NO: 49)
5′-GGTCTAAAGGACAGAAGACAACGGGAAGTGCCAATGTGCTCTTTCCAAGA
GCAAACACCCCGTTGGCTTCAAAGAAGTGGCAC-Nₓ-3′;
(artsgRNA12; SEQ ID NO: 50)
5′-GGTCTAAAGGACAGAAAATCTGTGCGTGTGCCATAAGTAATTAAAAATTAC
CCACCACAGGCTTCAAAGAAGTGGCAC-Nₓ-3′;
(artsgRNA13; SEQ ID NO: 51)
5′-GGTCGTCTATAGGACGGCGAGTTTTTCAACGGGTGTGCCAATGGCCACTTT
CCAGGTGGCAAAGCCCGTTGAGCTTCAAAGAAGTGGCAC-Nₓ-3′;
(artsgRNA14; SEQ ID NO: 52)
5′-GGTCGTCTATAGGACGGCGAGGACAACGGGAAGTGCCAATGTGCTCTTTCC
AAGAGCAAACACCCCGTTGGCTTCAAAGAAGTGGCAC-Nₓ-3′;
(artsgRNA15; SEQ ID NO: 53)
5′-GGTCGTCTATAGGACGGCGAGAATCTGTGCGTGTGCCATAAGTAATTAAAA
ATTACCCACCACAGGCTTCAAAGAAGTGGCAC-Nₓ-3′;
(artsgRNA16; SEQ ID NO: 54)
5′-GGTGACCTATAGGGTCAATGTTTTTCAACGGGTGTGCCAATGGCCACTTTCC
AGGTGGCAAAGCCCGTTGAGCTTCAAAGAAGTGGCAC-Nₓ-3′;
(artsgRNA17; SEQ ID NO: 55)
5′-GGTGACCTATAGGGTCAATGGACAACGGGAAGTGCCAATGTGCTCTTTCCA
AGAGCAAACACCCCGTTGGCTTCAAAGAAGTGGCAC-Nₓ-3′;
(artsgRNA18; SEQ ID NO: 56)
5′-GGTGACCTATAGGGTCAATGAATCTGTGCGTGTGCCATAAGTAATTAAAAAT
TACCCACCACAGGCTTCAAAGAAGTGGCAC-Nₓ-3′;
(artsgRNA19; SEQ ID NO: 57)
5′-GGTCTAAAGGACAGAATTTTTCAACGGGTGTGCCAATGGCCACTTTCCAGG
TGGCAAAGCCCGTTGAGATTATCTATGATGATTGGCAC-Nₓ-3′;
(artsgRNA20; SEQ ID NO: 58)
5′-GGTCTAAAGGACAGAAGACAACGGGAAGTGCCAATGTGCTCTTTCCAAGA
GCAAACACCCCGTTGGATTATCTATGATGATTGGCAC-Nₓ-3′;
(artsgRNA21; SEQ ID NO: 59)
5′-GGTCTAAAGGACAGAAAATCTGTGCGTGTGCCATAAGTAATTAAAAATTAC
CCACCACAGGATTATCTATGATGATTGGCAC-Nₓ-3′;
(artsgRNA22; SEQ ID NO: 60)
5′-GGTCGTCTATAGGACGGCGAGTTTTTCAACGGGTGTGCCAATGGCCACTTT
CCAGGTGGCAAAGCCCGTTGAGATTATCTATGATGATTGGCAC-Nₓ-3′;
(artsgRNA23; SEQ ID NO: 61)
5′-GGTCGTCTATAGGACGGCGAGGACAACGGGAAGTGCCAATGTGCTCTTTCC
AAGAGCAAACACCCCGTTGGATTATCTATGATGATTGGCAC-Nₓ-3′;
(artsgRNA24; SEQ ID NO:62)
5′-GGTCGTCTATAGGACGGCGAGAATCTGTGCGTGTGCCATAAGTAATTAAAA
ATTACCCACCACAGGATTATCTATGATGATTGGCAC-Nₓ-3′;
(artsgRNA25; SEQ ID NO: 63)
5′-GGTGACCTATAGGGTCAATGTTTTTCAACGGGTGTGCCAATGGCCACTTTCC
AGGTGGCAAAGCCCGTTGAGATTATCTATGATGATTGGCAC-Nₓ-3′;
(artsgRNA26; SEQ ID NO: 64)
5′-GGTGACCTATAGGGTCAATGGACAACGGGAAGTGCCAATGTGCTCTTTCCA
AGAGCAAACACCCCGTTGGATTATCTATGATGATTGGCAC-Nₓ-3′;
(artsgRNA27; SEQ ID NO: 65)
5′-GGTGACCTATAGGGTCAATGAATCTGTGCGTGTGCCATAAGTAATTAAAAAT
TACCCACCACAGGATTATCTATGATGATTGGCAC-Nₓ-3′;
(artsgRNA28; SEQ ID NO: 66)
5′-GGTCTAAAGGACAGAACAACGGGATGTGCCAATGCACTCTTTCCAGGAGT
GAACACCCCGTTGACTTCAAGCGAAGTGGCAC-Nₓ-3′;
(artsgRNA29; SEQ ID NO: 67)
5′-GGTCGTCTATAGGACGGCGAGCAACGGGATGTGCCAATGCACTCTTTCCAG
GAGTGAACACCCCGTTGACTTCAAGCGAAGTGGCAC-Nₓ-3′;
(artsgRNA30; SEQ ID NO: 68)
5′-GGAATTGCCGATCTATAGGACGGCAGATTTTTTTCAACGGGTGTGCCAATG
GCCACTTTCCAGGTGGCAAAGCCCGTTGAACTTCAAGCGAAGTGGCAC-Nₓ-3′;
(artsgRNA31; SEQ ID NO: 69)
5′-GGAATTGCCGATCTATAGGACGGCAGATTGACAACGGGAAGTGCCAATGTG
CTCTTTCCAAGAGCAAACACCCCGTTGACTTCAAGCGAAGTGGCAC-Nₓ-3′;
(artsgRNA32; SEQ ID NO: 70)
5′-GGAATTGCCGATCTATAGGACGGCAGATTCAACGGGATGTGCCAATGCACT
CTTTCCAGGAGTGAACACCCCGTTGACTTCAAGCGAAGTGGCAC-Nₓ-3′;
(artsgRNA33; SEQ ID NO: 71)
5′-GGTCTAAAGGACAGAACAACGGGATGTGCCAATGCACTCTTTCCAGGAGT
GAACACCCCGTTGGCTTCAAAGAAGTGGCAC-Nₓ-3′;
(artsgRNA34; SEQ ID NO: 72)
5′-GGTCGTCTATAGGACGGCGAGCAACGGGATGTGCCAATGCACTCTTTCCAG
GAGTGAACACCCCGTTGGCTTCAAAGAAGTGGCAC-Nₓ-3′;
(artsgRNA35; SEQ ID NO: 73)
5′-GGAATTGCCGATCTATAGGACGGCAGATTTTTTTCAACGGGTGTGCCAATG
GCCACTTTCCAGGTGGCAAAGCCCGTTGAGCTTCAAAGAAGTGGCAC-Nₓ-3′;
(artsgRN36A; SEQ ID NO: 74)
5′-GGAATTGCCGATCTATAGGACGGCAGATTGACAACGGGAAGTGCCAATGTG
CTCTTTCCAAGAGCAAACACCCCGTTGGCTTCAAAGAAGTGGCAC-Nₓ-3′;
or
(artsgRNA37; SEQ ID NO: 75)
5′-GGAATTGCCGATCTATAGGACGGCAGATTCAACGGGATGTGCCAATGCACT
CTTTCCAGGAGTGAACACCCCGTTGGCTTCAAAGAAGTGGCAC-Nₓ-3′,

wherein Nx represents nucleotide sequence that consists of X consecutive nucleotides(spacer sequence), N is independently selected from A, G, C and T; X is an integer of 18 [symbol] X [symbol] 35, preferably, X=20. In some embodiments, the sequence Nx (spacer sequence) is capable of specifically hybridizing to the complement of the target sequence. The sequence other than Nx in the sgRNA is a scaffold sequence of sgRNA.

In some embodiments, the artificial sgRNA comprises a scaffold sequence encoded by the nucleotide sequence of any one of SEQ ID NOs:39-75.

In some embodiments, the guide RNA in the genome editing system of the invention is an artificial sgRNA of the invention.

To achieve efficient expression in target cells, in some embodiments of the invention, the nucleotide sequence encoding a C2c1 protein or variant thereof is codon optimized for the organism from which the cell to be genome edited is derived.

Codon optimization refers to the replacement of at least one codon (e.g., about or more than about 1, 2, 3, 4, 5, 10, 15, 20, 25, 50 or more codons) of a native sequence by a codon that is used more frequently or most frequently in the gene of the host cell, modifying the nucleic acid sequence while maintaining the native amino acid sequence to enhance expression in the host cell of interest. Different species show specific preferences for certain codons of a particular amino acid. Codon preference (difference in codon usage between organisms) is often associated with the efficiency of translation of messenger RNA (mRNA), which is believed to depend on the nature of the translated codon and the availability of specific transfer RNA (tRNA) molecules. The advantages of selected tRNAs within cells generally reflect the most frequently used codons for peptide synthesis. Therefore, genes can be customized to be best gene expressed in a given organism based on codon optimization. The codon usage table can be easily obtained, for example, in the Kazusa Codon Usage Database available on the World Wide Web, and these tables can be adjusted in different ways. See, Nakamura Y. et. al. “Codon usage tabulated from the international DNA sequence databases: status for the year 2000 Nucl. Acids Res, 28: 292 (2000).

The organism from which the cell can be genome-edited by the system of the present invention preferably is eukaryote, including but not limited to mammals such as human, mouse, rat, monkey, dog, pig, sheep, cattle, cat; poultry such as chicken, duck, goose; plants including monocots and dicots, such as rice, corn, wheat, sorghum, barley, soybean, peanut and Arabidopsis thaliana and so on.

In some embodiments of the invention, the nucleotide sequence encoding a C2c1 protein or variant thereof is codon optimized for human. In some embodiments, the codon-optimized nucleotide sequence encoding AaC2c1, AkC2c1, AmC2c1, BhC2c1, BsC2c1, Bs3C2c1, DiC2c1, LsC2c1, SbC2c1, TcC2c1 protein is selected from SEQ ID NOs: 11-20, respectively.

In some embodiments of the present invention, the nucleotide sequence encoding the C2c1 protein or variant thereof and/or the nucleotide sequence encoding the guide RNA is operably linked to an expression regulatory element such as a promoter.

As used in the present invention, “expression construct” refers to a vector such as a recombinant vector that is suitable for expression of a nucleotide sequence of interest in an organism. “Expression” refers to the production of a functional product. For example, expression of a nucleotide sequence may refer to the transcription of a nucleotide sequence (e.g., transcription to produce mRNA or functional RNA) and/or the translation of RNA into a precursor or mature protein. The “expression construct” of the present invention may be a linear nucleic acid fragment, a circular plasmid, a viral vector or, in some embodiments, an RNA that is capable of translation (such as mRNA).

The “expression construct” of the present invention may comprise regulatory sequences and nucleotide sequences of interest from different origins, or regulatory sequences and nucleotide sequences of interest from the same source but arranged in a manner different from that normally occurring in nature.

“Regulatory sequence” and “regulatory element” are used interchangeably to refer to a nucleotide sequence that is located upstream (5′ non-coding sequence), middle or downstream (3′ non-coding sequence) of a coding sequence and affects the transcription, RNA processing or stability or translation of the relevant coding sequence. Regulatory sequences may include, but are not limited to, promoters, translation leaders, introns and polyadenylation recognition sequences.

“Promoter” refers to a nucleic acid fragment capable of controlling the transcription of another nucleic acid fragment. In some embodiments of the present invention, the promoter is a promoter capable of controlling the transcription of a gene in a cell, whether or not it is derived from the cell. The promoter may be a constitutive promoter or tissue-specific promoter or developmentally-regulated promoter or inducible promoter.

“Constitutive promoter” refers to a promoter that may in general cause the gene to be expressed in most cases in most cell types. “Tissue-specific promoter” and “tissue-preferred promoter” are used interchangeably and mean that they are expressed primarily but not necessarily exclusively in one tissue or organ, but also in a specific cell or cell type. “Developmentally-regulated promoter” refers to a promoter whose activity is dictated by developmental events. “Inducible promoter” selectively expresses operably-linked DNA sequences in response to an endogenous or exogenous stimulus (environment, hormones, chemical signals, etc.).

As used herein, the term “operably linked” refers to the linkage of a regulatory element (e.g., but not limited to, a promoter sequence, a transcription termination sequence, etc.) to a nucleic acid sequence (e.g., a coding sequence or an open reading frame) such that transcription of the nucleotide sequence is controlled and regulated by the transcriptional regulatory element. Techniques for operably linking regulatory element regions to nucleic acid molecules are known in the art.

Examples of promoters that can be used in the present invention include, but are not limited to, the polymerase (pol) I, pol II or pol III promoters. Examples of the pol I promoter include the gallus RNA pol I promoter. Examples of the pol II promoters include, but are not limited to, the immediate-early cytomegalovirus (CMV) promoter, the Rous sarcoma virus long terminal repeat (RSV-LTR) promoter, and the immediate-early simian virus 40 (SV40) promoter. Examples of pol III promoters include the U6 and H1 promoters. An inducible promoter such as a metallothionein promoter can be used. Other examples of promoters include the T7 phage promoter, the T3 phage promoter, the β-galactosidase promoter, and the Sp6 phage promoter, and the like. Promoters that can be used in plants include, but are not limited to, cauliflower mosaic virus 35S promoter, maize Ubi-1 promoter, wheat U6 promoter, rice U3 promoter, maize U3 promoter, rice actin promoter.

The cell that can be edited by the method of the present invention preferably is an eukaryotic cell, including but not limited to a mammalian cell such as a cell of human, mouse, rat, monkey, dog, pig, sheep, cattle, cat; a cell of poultry such as chicken, duck, goose; a cell of plants including monocots and dicots, such as rice, corn, wheat, sorghum, barley, soybean, peanut and Arabidopsis thaliana and so on. In some embodiments of the invention, the cell is a eukaryotic cell, preferably a mammalian cell, more preferably a human cell.

In another aspect, the present invention provides a method of modifying the target sequence in cell genome, comprising introducing the genome editing system of the invention into the cell, whereby the guide RNA targets the C2c1 protein or variant thereof to a target sequence in the genome of the cell. In some embodiments, the targeting results in one or more nucleotides being substituted, deleted and/or added in the target sequence.

“Introduction” of a nucleic acid molecule (e.g., plasmid, linear nucleic acid fragment,

RNA, etc.) or protein of the invention into a cell means that the nucleic acid or protein is used to transform a cell such that the nucleic acid or protein is capable of functioning in the cell. As used in the present invention, “transformation” includes both stable and transient transformations. “Stable transformation” refers to the introduction of exogenous nucleotide sequence into the genome, resulting in the stable inheritance of foreign sequence. Once stably transformed, the exogenous nucleic acid sequence is stably integrated into the genome of the organism and any of its successive generations. “Transient transformation” refers to the introduction of a nucleic acid molecule or protein into a cell, executing its function without the stable inheritance of an exogenous sequence. In transient transformation, the exogenous nucleic acid sequence is not integrated into the genome.

Methods that can be used to introduce the genome editing system of the present invention into a cell include, but are not limited to, calcium phosphate transfection, protoplast fusion, electroporation, lipofection, microinjection, viral infection (e.g., baculovirus, vaccinia virus, adenovirus, adeno-associated virus, lentivirus and other viruses), gene gun method, PEG-mediated protoplast transformation, Agrobacterium-mediated transformation.

In some embodiments, the method of the invention is performed in vitro. For example, the cell is an isolated cell. In some embodiments, the cell is a CAR-T cell. In some embodiments, the cell is an induced embryonic stem cell.

In other embodiments, the method of the invention may also be performed in vivo. For example, the cell is a cell within an organism, and the system of the present invention can be introduced into the cell in vivo by, for example, a virus-mediated method. For example, the cell can be a tumor cell within a patient.

In another aspect, the present invention provides a method of producing a genetically modified cell, comprising introducing the genome editing system of the present invention into a cell, whereby the guide RNA targets the C2c1 protein or variant thereof to a target sequence in the genome of the cell. In some embodiments, the targeting results in one or more nucleotides being substituted, deleted and/or added in the target sequence.

In another aspect, the invention also provides a genetically modified organism comprising a genetically modified cell produced by the methods of the invention or a progeny cell thereof.

As used herein, “organism” includes any organism that is suitable for genome editing, eukaryotes are preferred. Examples of the organism include, but are not limited to, mammals such as human, mouse, rat, monkey, dog, pig, sheep, cattle, cat; poultry such as chicken, duck, goose; plants including monocots and dicots such as rice, corn, wheat, sorghum, barley, soybean, peanut, Arabidopsis and the like. In some embodiments of the invention, the organism is eukaryote, preferably a mammal, and more preferably a human.

A “genetically modified organism” or “genetically modified cell” includes the organism or the cell which comprises within its genome an exogenous polynucleotide or a modified gene or modified expression regulatory sequence. For example, the exogenous polynucleotide is stably integrated within the genome of the organism or the cell such that the polynucleotide is passed on to successive generations. The exogenous polynucleotide may be integrated into the genome alone or as part of a recombinant DNA construct. The modified gene or modified expression regulatory sequence means that, in the genome of the organism or the cell, said sequence comprises one or more nucleotide substitution, deletion, or addition. “Exogenous” in reference to a sequence means a sequence from a foreign species, or refers to a sequence in which significant changes in composition and/or locus occur from its native form through deliberate human intervention if from the same species.

In another aspect, the invention provides a gene expression regulatory system based on a nuclease-dead C2c1 protein of the invention. This system, although not changing the sequence of the target gene, is also defined as a genome editing system within the scope herein.

In some embodiments, the gene expression regulation system is a gene suppressing or silencing system, comprising one of the following:

i) a nuclease-dead C2c1 protein or a fusion protein of the nuclease-dead C2c1 protein and a transcriptional repressor protein, and a guide RNA;

ii) an expression construct comprising a nucleotide sequence encoding a nuclease-dead C2c1 protein or a fusion protein of the nuclease-dead C2c1 protein and a transcriptional repressor protein, and a guide RNA;

iii) a nuclease-dead C2c1 protein or a fusion protein of the nuclease-dead C2c1 protein and a transcriptional repressor protein, and an expression construct comprising a nucleotide sequence encoding a guide RNA;

iv) an expression construct comprising a nucleotide sequence encoding a nuclease-dead C2c1 protein or a fusion protein of the nuclease-dead C2c1 protein and a transcriptional repressor protein, and an expression construct comprising a nucleotide sequence encoding a guide RNA; or

v) an expression construct comprising a nucleotide sequence encoding a nuclease-dead C2c1 protein or a fusion protein of the nuclease-dead C2c1 protein and a transcriptional repressor protein and a nucleotide sequence encoding a guide RNA.

The definition of the nuclease-dead C2c1 protein or the guide RNA is as described above. Selection of the transcriptional repressor protein is within the skill of those ordinary people in the art.

As used herein, gene suppression or silencing refers to the down-regulation or elimination of gene expression, preferably at the transcriptional level.

However, the gene expression regulatory system of the present invention can also use a fusion protein of a nuclease-dead C2c1 protein and a transcriptional activator protein. In this case, the gene expression regulatory system is a gene expression activation system. For example, the gene expression activation system of the present invention may comprise one of the following:

i) a fusion protein of a nuclease-dead C2c1 protein and a transcriptional activator protein, and a guide RNA;

ii) an expression construct comprising a nucleotide sequence encoding a fusion protein of a nuclease-dead C2c1 protein and a transcriptional activator protein, and a guide RNA;

iii) a fusion protein of a nuclease-dead C2c1 protein and a transcriptional activator protein, and an expression construct comprising a nucleotide sequence encoding a guide RNA;

iv) an expression construct comprising a nucleotide sequence encoding a fusion protein of a nuclease-dead C2c1 protein and a transcriptional activator protein, and an expression construct comprising a nucleotide sequence encoding a guide RNA; or

v) an expression construct comprising a nucleotide sequence encoding a fusion protein of a nuclease-dead C2c1 protein and a transcriptional activator protein and a nucleotide sequence encoding a guide RNA.

The definition of the nuclease-dead C2c1 protein or the guide RNA is as described above. Selection of the transcriptional activator protein is within the skill of those ordinary people in the art.

As used herein, gene activation refers to up-regulation of gene expression levels, preferably at the transcriptional level.

In another aspect, the invention also encompasses the use of the genome editing system of the invention in the treatment of diseases.

By modifying a disease-related gene by the genome editing system of the present invention, it is possible to achieve up-regulation, down-regulation, inactivation, activation, or mutation correction of the disease-related gene, thereby achieving prevention and/or treatment of the disease. For example, in the present invention, the target sequence may be located in the protein coding region of the disease-related gene, or may be, for example, located in a gene expression regulatory region such as a promoter region or an enhancer region, thereby enabling functional modification of the disease-related gene or modification of the expression of the disease-related gene.

A “disease-related” gene refers to any gene that produces a transcriptional or translational product at an abnormal level or in an abnormal form in a cell derived from a disease-affected tissue as compared to a tissue or cell of non-disease control. When altered expression is related with the appearance and/or progression of a disease, it may be a gene that is expressed at an abnormally high level or it may be a gene that is expressed at an abnormally low level. A disease-related gene also refers to a gene having one or more mutations or a genetic variation that is directly responsible for or has genetic linkage with one or more genes responsible for the etiology of the disease. The transcribed or translated product may be known or unknown and may be at normal or abnormal levels.

Accordingly, in another aspect, the invention also provides a method of treating a disease in a subject in need thereof, comprising delivering to the subject an effective amount of a genome editing system of the invention to modify a gene related to the disease.

In another aspect, the invention also provides the use of a genome editing system of the invention for the preparation of a pharmaceutical composition for treating a disease in a subject in need thereof, wherein the genome editing system is for modifying a gene related to the disease.

In another aspect, the invention also provides a pharmaceutical composition for treating a disease in a subject in need thereof, comprising a genome editing system of the invention and a pharmaceutically acceptable carrier, wherein the genome editing system is for modifying a gene related to the disease.

In some embodiments, the subject is a mammal, such as a human.

Examples of such diseases include, but are not limited to, tumors, inflammation, Parkinson's disease, cardiovascular disease, Alzheimer's disease, autism, drug addiction, age-related macular degeneration, schizophrenia, hereditary diseases, and the like.

In another aspect, the invention also includes a kit for use in the methods of the invention, the kit comprising the genome editing system of the invention, and an instruction. The kits generally include a label indicating the intended use and/or method of use of the contents in the kit. The term label includes any written or recorded material provided on or with the kit or otherwise provided with the kit.

### EXAMPLES

In order to facilitate the understanding of the present invention, the present invention will be described more fully hereinafter with reference to the specific examples and the accompanying drawings. Preferred embodiments of the invention are shown in the drawings. However, the invention may be embodied in many different forms and is not limited to the embodiments described herein. Rather, these embodiments are provided so that the present disclosure will be more fully understood.

### Materials and Methods

### 1. DNA Manipulations.

DNA manipulation including DNA preparation, digestion, ligation, amplification, synthesis, purification, agarose gel electrophoresis, etc. were conducted according to Molecular Cloning: A Laboratory Manual with some modifications. Briefly, targeting chimeric single guide RNA (sgRNA) scaffolds for cell transfection assay were constructed by ligation annealed oligonucleotides (oligos) (Table 1) into BsaI-digested pUC19-U6-sgRNA (SEQ ID NO:23-30) vectors.

TABLE 1
Target sequences of human genomic targets.

| Gene | Protospacer ID | Protospacer sequences (5′-3′) | 5′PAM | Strand |
|---|---|---|---|---|
| CCR5 | CCR5 site 10 | TCCTTCTCCTGAACACCTTC | TTG | + |
| | | (SEQ ID NO: 76) | | |
| | CCR5 site 28 | TTTGGCCTGAATAATTGCAG | TTC | + |
| | | (SEQ ID NO: 77) | | |
| DNMT1 | DNMT1 site 16 | CCCTTCAGCTAAAATAAAGG | TTT | + |
| | | (SEQ ID NO: 78) | | |
| RNF2 | RNF2 site 8 | TAGTCATGGTGTTCTTCAAC | TTG | − |
| | | (SEQ ID NO: 79) | | |
| — | Site 5 | GCTCTCAAGACCCACAATCC | TTT | + |
| | | (SEQ ID NO: 80) | | |

### 2. De Novo Gene Synthesis and Plasmid Construction.

PSI-BLAST program (Altschul, S. F. et al. Nucleic Acids Res 25, 3389-3402 (1997)) was adopted to identify new CRISPR-C2c1 orthologs. Their coding sequences were humanized (Grote, A. et al., Nucleic Acids Res 33, W526-531 (2005)) and oligos for C2c1 gene and sgRNA synthesis were designed using GeneDesign program (Richardson, S. M. et al., Genome Res 16, 550-556 (2006)). Each C2c1 gene was synthesized according to article (Li, G. et al., Methods Mol Biol 1073, 9-17 (2013)). Purified products were assembled into expression vectors via homologous recombination in vitro using NEBuilder® HiFi DNA Assembly Master Mix (NEB). The pCAG-2AeGFP (SEQ ID NO:21) and pUC19-U6 vectors (SEQ ID NO:22) were applied for mammalian expression of C2c1 proteins and sgRNAs, respectively.

### 3. Cell Culture and Transfection.

Human embryonic kidney 293T cells were cultured in Dulbecco's Modified Eagle's Medium (DMEM, Gibco) supplemented with 10% fetal bovine serum (FBS, Gibco) and 1% Antibiotic-Antimycotic (Gibco) at 37° C. with 5% CO2 incubation. 293T cells were transfected using Lipofectamine LTX (Invitrogen) following the manufacturer's recommended protocol. For each well of a 48-well plate, a total of 400 ng plasmid (C2c1: sgRNA=2:1) were used. Then 48 h following transfection, cells were harvested directly for genomic DNA extraction.

### 4. T7 Endonuclease I (T7EI) Assay and Sanger Sequencing.

Harvested cells were lysed directly with Buffer L (Bimake) supplemented with Protease K and incubated at 55° C. for 3 h and inactivated at 95° C. for 10 min. Genomic region surrounding the C2c1 target site for each gene was PCR-amplified (Supplementary Table 5). 200˜400 ng PCR products were mixed with ddH2O to a final volume of 10 μL, and subjected to re-annealing process to enable heteroduplex formation according to previous methods. After re-annealing, products were treated with 1/10 volume of NEBuffer™M 2.1 and 0.2 μL T7EI (NEB) at 37° C.for 30 min, and analyzed on 3% agarose gels. Indels were quantitated based on relative band intensities (Cong, L. et al., Science 339, 819-823 (2013)). T7EI assay identified mutated products were subjected to be cloned into TA cloning vector and transformed into competent E. coli strain (Transgen Biotech). After overnight culture, colonies were randomly picked out and sequenced.

### Example 1. New C2c1 Proteins Idenfication

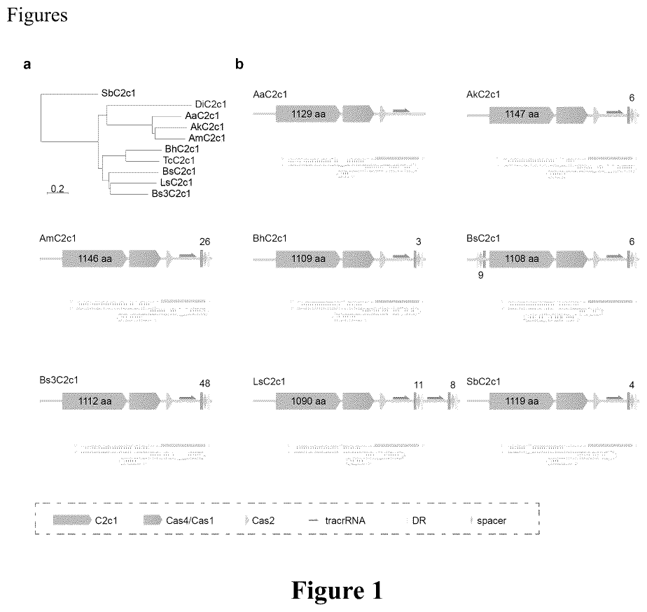

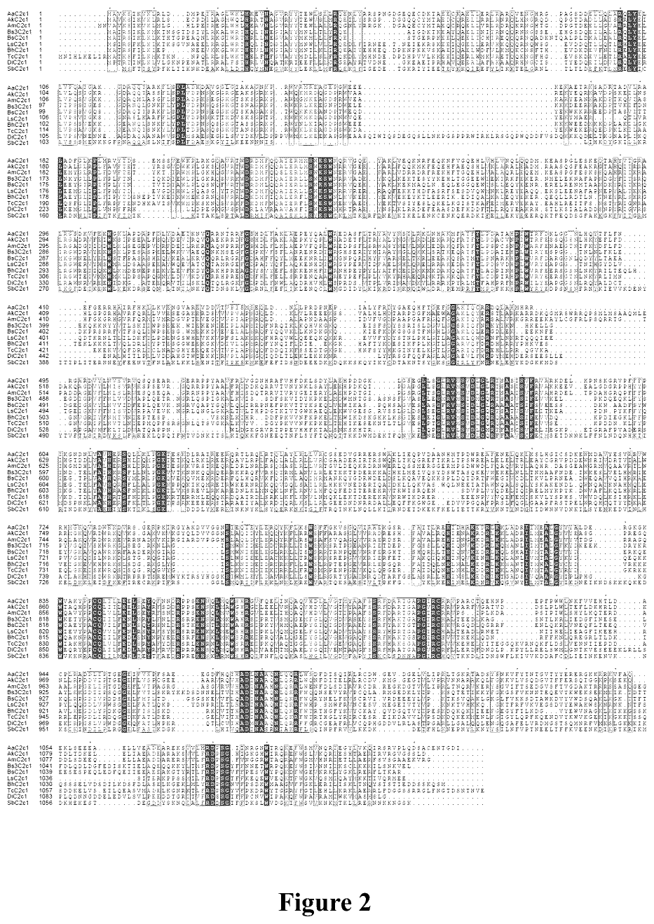

Six representative C2c1-family proteins from diverse bacteria as well as four previously reported C2c1 orthologs were selected, and de novo synthesized to conduct genome editing in human embryonic kidney 293T cells (FIGS. 1 and 2 and SEQ ID NO:1-10). Among these ten C2c1 orthologs, C2c1 from D. inopinatus (DiC2cl) and T. calidus (TcC2c1) have neither predicted precursor CRISPR RNA (pre-crRNA) nor the reverse-repeated trans-activating crRNA (tracrRNA) (FIG. 1B), suggesting that these two C2c1 proteins might be unsuitable for genome-editing applications.

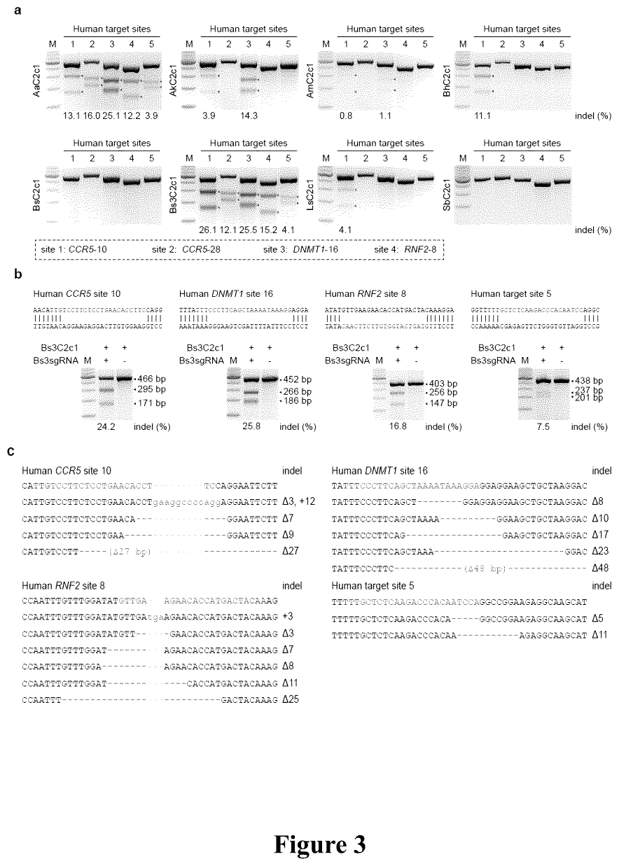

To conduct mammalian genome editing, we co-transfected 293T cells with individual C2c1 enzymes and their cognate chimeric single guide RNAs (sgRNAs) targeting human endogenous loci containing appropriate PAMs (FIGS. 1A and 1B). Results of T7 Endonuclease (T7EI) assay showed that apart from previously reported AaC2c1 and AkC2c1, four novel C2c1 orthologs (AmC2c1, BhC2c1, Bs3C2c1 and LsC2c1) could robustly edit the human genome, though their targeting efficiencies varied among different orthologs and at different targeting sites (FIGS. 1B and 3A). We also achieved multiplex genome engineering by programming Bs3C2c1 to simultaneously edit four sites in the human genome by simply using multiple sgRNAs (FIGS. 3B and 3C). These newly-identified C2c1 orthologs expand our options for C2c1-based genome engineering.

### Example 2. Exchangeability Between Different C2c1 and Ddual-RNA

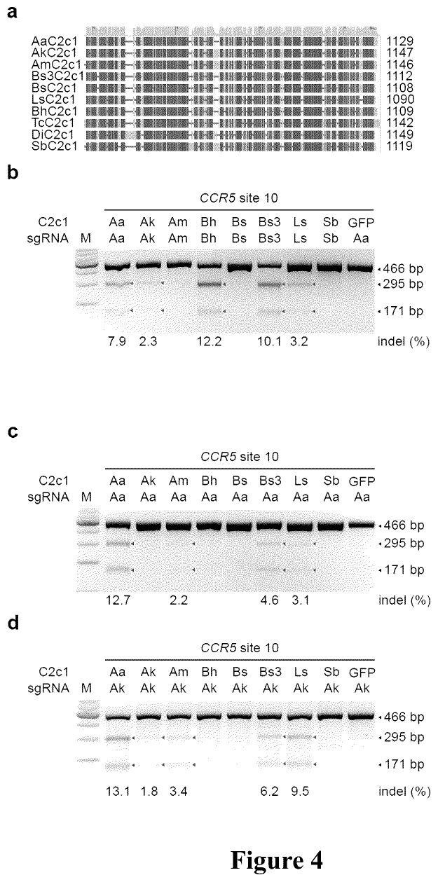

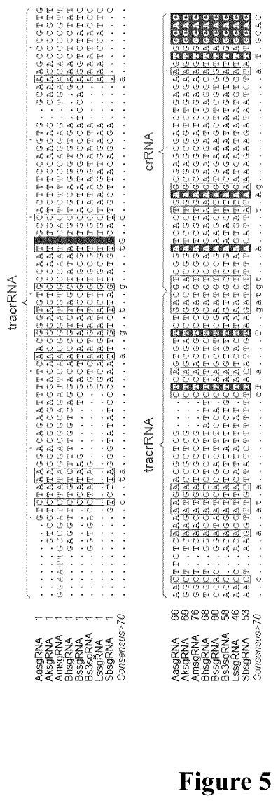

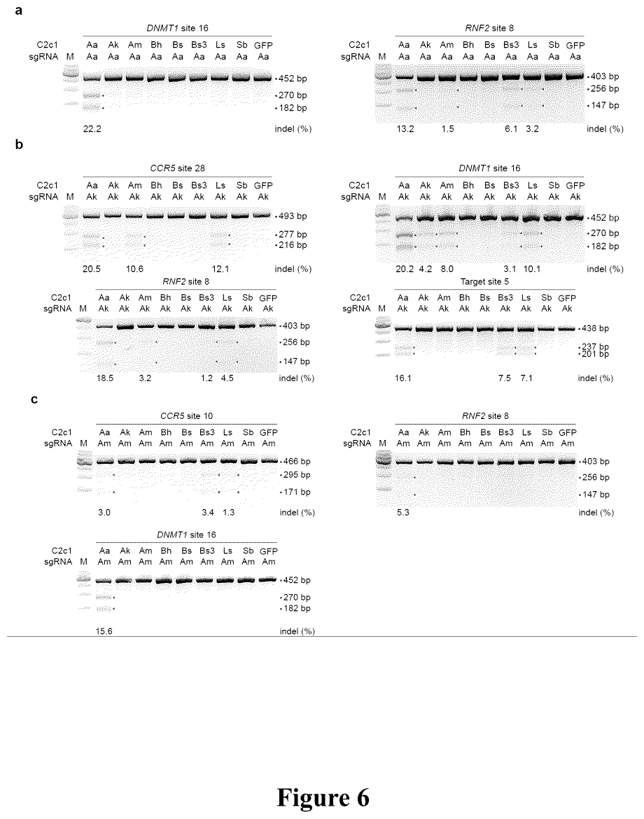

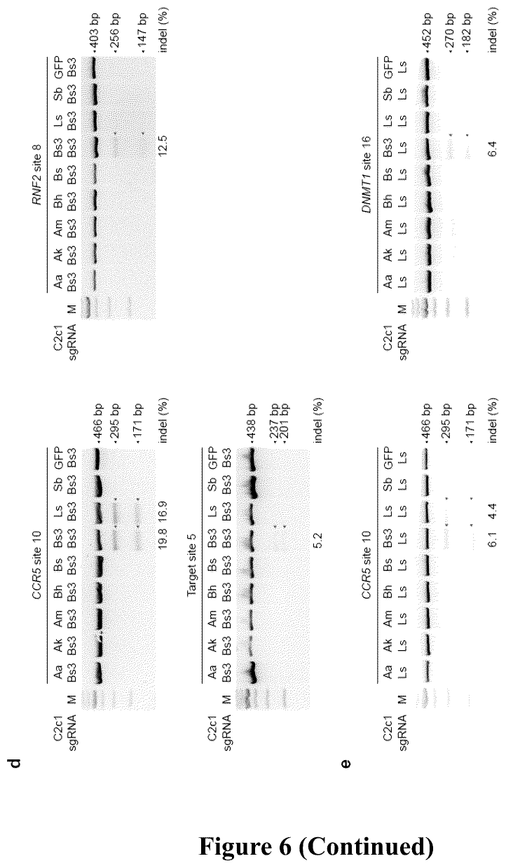

To investigate the interchangeability between the dual-RNA and protein components in C2c1 systems, we first analyze the conservation of both C2c1 proteins and dual-RNAs. Besides the conserved amino acid sequences of C2c1 orthologs (FIGS. 4A and 2), the DNA sequences of pre-crRNA:tracrRNA duplex and their secondary structures also exhibited high conservation (FIGS. 1B and 5). Next, we conducted genome editing in 293T cells with the eight C2c1 orthologs complexed with respective sgRNAs from the eight C2c1 systems separately. As results of T7EI assay indicated, sgRNAs derived from AaC2c1, AkC2c1, AmC2c1, Bs3C2c1 and LsC2c1 loci could substitute the original sgRNAs for mammalian genome editing, though their activities varied between different C2c1 orthologs and sgRNAs (FIGS. 4C, 4D, and 6A-6F). These results demonstrated the exchangeability between C2c1 and dual-RNA from different C2c1 loci.

### Example 3. Using the C2c1 Ortholog With No CRISPR Array In Natural Loci For Genome Editing

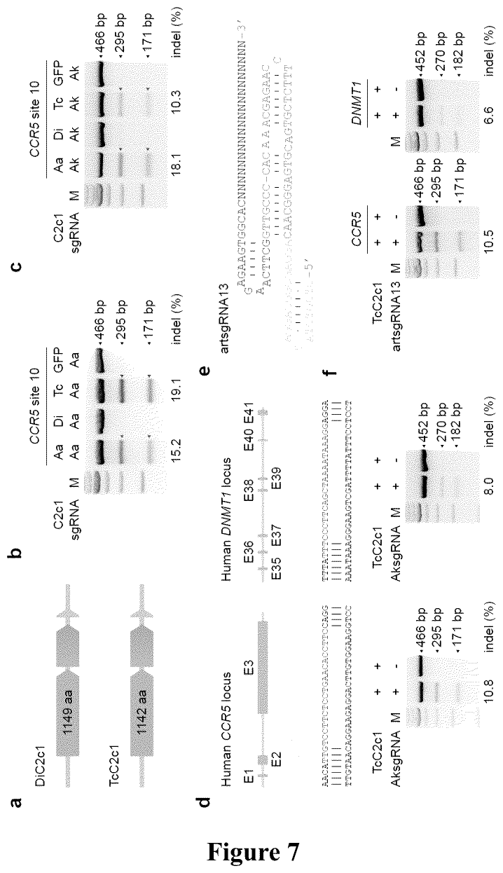

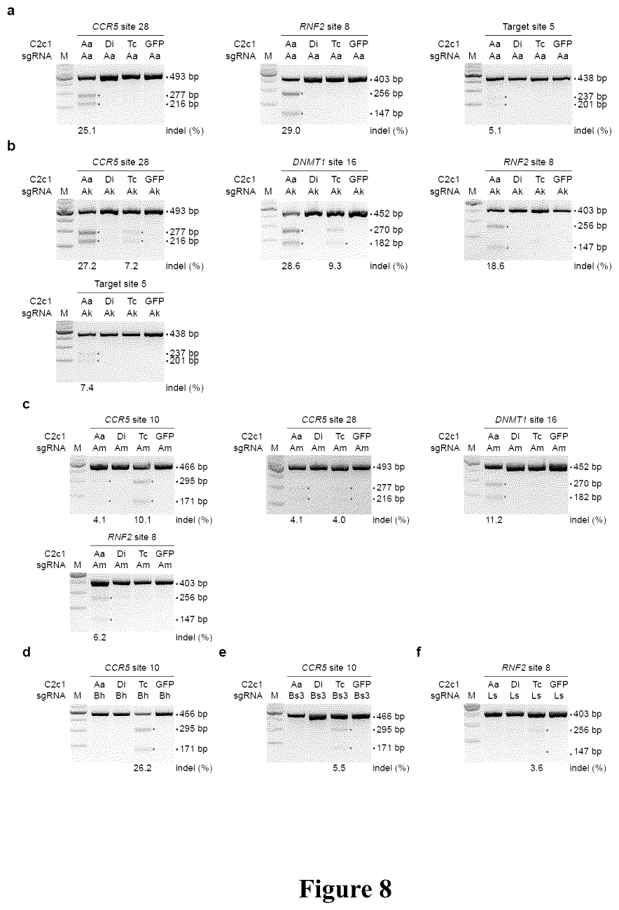

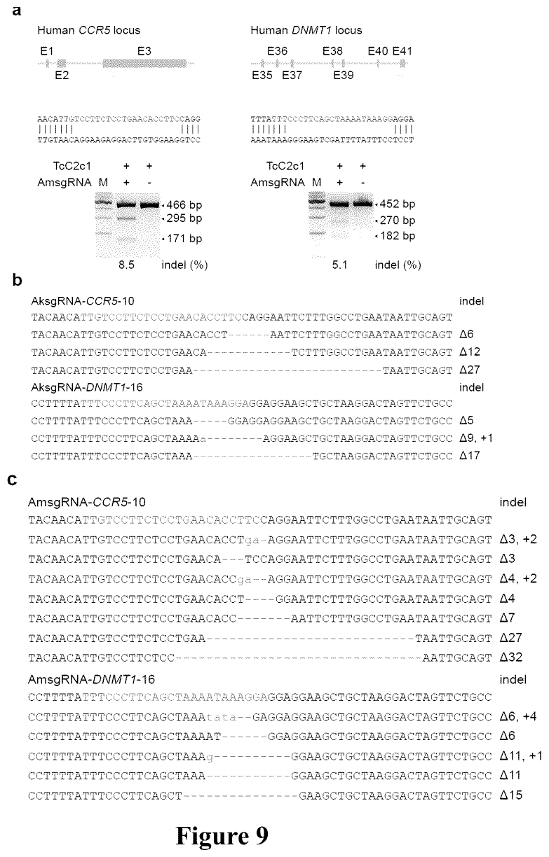

To further demonstrate our hypothesis, we chose two C2c1 orthologs (DiC2c1 and TcC2c1) whose loci harbored no CRISPR array (FIG. 7A), making the sequences of their crRNA:tracrRNA duplex unpredictable, to conduct subsequent experiments. We co-transfected DiC2c1 and TcC2c1 as well as AaC2c1 combined with the sgRNAs derived from loci of other eight C2c1 orthologs targeting different genomic sites in 293T cells. T7EI assay results indicated that sgRNAs derived from AaC2c1, AkC2c1, AmC2c1, Bs3C2c1 and LsC2c1 could enable TcC2c1 to robustly edit the human genome (FIGS. 7B, 7C, and 8A-8F). Furthermore, TcC2c1 could facilitate multiplex genome editing simultaneously directed by either AasgRNAs or AksgRNAs (FIGS. 7D and 9A-9C). These above results demonstrated that the interchangeability between C2c1 and dual-RNA from different systems could empower the C2c1 ortholog with no CRISPR array in natural loci to engineer the mammalian genomes.

### Example 4. Design Artificial sgRNA for C2c1-Mediated Genome Editing

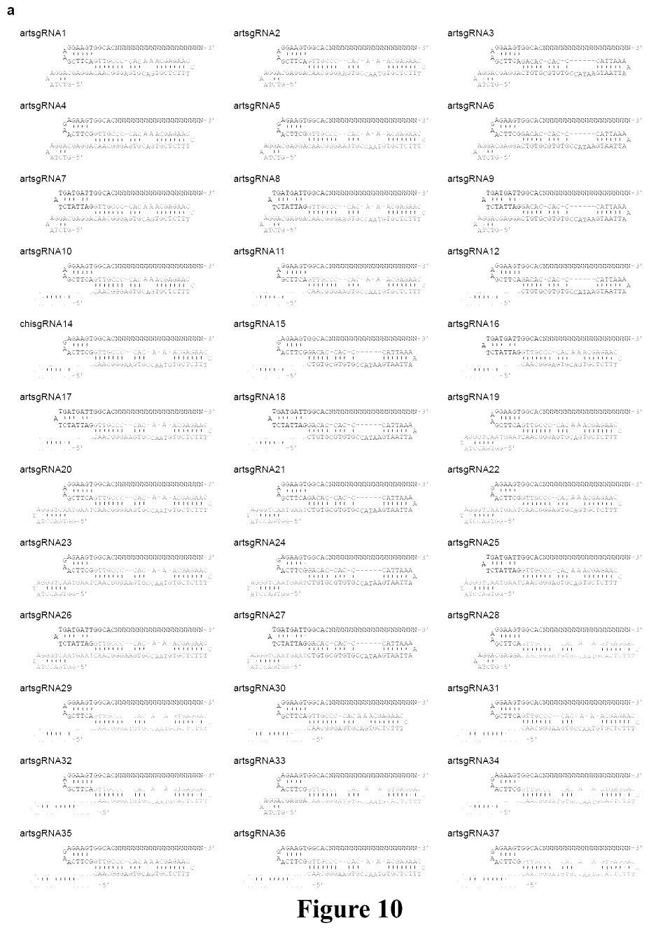

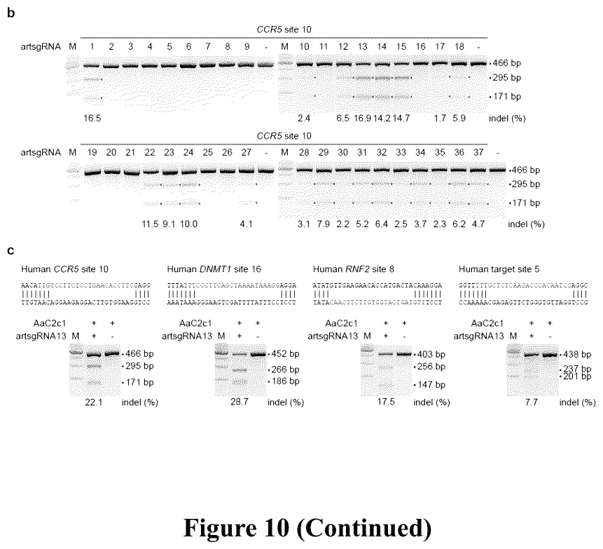

The exchangeability between C2c1 and dual-RNA in different C2c1 systems further promoted us to design novel artificial sgRNA (artsgRNA) scaffolds to facilitate C2c1-mediated genome editing. Considering the conservation of DNA sequences and secondary structure among C2c1 orthologs (FIGS. 1B and 3A-3C), we designed and de novo synthesized 37 sgRNA scaffolds targeting the human CCR5 locus (FIGS. 7E and 10A). The results of T7EI assay suggested that 22 artsgRNA scaffolds could work efficiently (FIG. 10B). To verify the generalization of our artsgRNAs, we leveraged multiplex genome editing using TcC2c1 or AaC2c1 directed by artsgRNA13 (FIG. 10A). T7EI assay results indicated that artsgRNA13 could simultaneously facilitate multiplex genome engineering with both TcC2c1 and AaC2c1 (FIG. 7F and 10C). Our results suggested that we could design and synthesize artsgRNAs to facilitate C2c1-mediated multiplex genome editing.

TABLE 2
Sequences and information involved in the present invention

| SEQ ID NO | Information |
|---|---|
| SEQ ID NO: 1 | AaC2c1 amino acid sequence from Alicyclobacillus acidiphilus NBRC |
| | 100859 (GeneBank ID: NZ_BCQI01000053.1) |
| SEQ ID NO: 2 | AkC2c1 amino acid sequence from Alicyclobacillus kakegawensis NBRC |
| | 103104 (GeneBank ID: NZ_BCRP01000027.1) |
| SEQ ID NO: 3 | AmC2c1 amino acid sequence from Alicyclobacillus macrosporangiidus |
| | strain DSM 17980 (GeneBank ID: NZ_FPBV01000001.1) |
| SEQ ID NO: 4 | BhC2c1 amino acid sequence from Bacillus hisashii strain C4 |
| | (GeneBank ID: NZ_NJGA01000060.1) |
| SEQ ID NO: 5 | BsC2c1 amino acid sequence from Bacillus sp. NSP2.1 (GeneBank ID: |
| | NZ_KI301973.1) |
| SEQ ID NO: 6 | Bs3C2c1 amino acid sequence from Bacillus sp. V3-13 contig_40 (GeneBank |
| | ID: NZ_PGUZ01000040.1) |
| SEQ ID NO: 7 | DiC2c1 amino acid sequence from Desulfovibrio inopinatus DSM 10711 |
| | (GeneBank: NZ_KE386879.1) |
| SEQ ID NO: 8 | LsC2c1 amino acid sequence from Laceyella sediminis strain RHA1 |
| | (GeneBank ID: NZ_PVTZ01000002.1) |
| SEQ ID NO: 9 | SbC2c1 amino acid sequence from Spirochaetes bacterium GWB1_27_13 |
| | (GeneBank ID: MIAN01000063.1) |
| SEQ ID NO: 10 | TcC2c1 amino acid sequence from Tuberibacillus calidus DSM 17572 |
| | (GeneBank ID: NZ_KE387196.1) |
| SEQ ID NO: 11 | Humanized AaC2c1 coding sequence from Alicyclobacillus acidiphilus |
| | NBRC 100859 (GeneBank ID: NZ_BCQI01000053.1) |
| SEQ ID NO: 12 | Humanized AkC2c1 coding sequence from Alicyclobacillus kakegawensis |
| | NBRC 103104 (GeneBank ID: NZ_BCRP01000027.1) |
| SEQ ID NO: 13 | Humanized AmC2c1 coding sequence from Alicyclobacillus |
| | macrosporangiidus strain DSM 17980 (GeneBank ID: |
| | NZ_FPBV01000001.1) |
| SEQ ID NO: 14 | Humanized BhC2c1 coding sequence from Bacillus hisashii strain C4 |
| | (GeneBank ID: NZ_NJGA01000060.1) |
| SEQ ID NO: 15 | Humanized BsC2c1 coding sequence from Bacillus sp. NSP2.1 |
| | (GeneBank ID: NZ_KI301973.1) |
| SEQ ID NO: 16 | Humanized Bs3C2c1 coding sequence from Bacillus sp. V3-13 contig_40 |
| | (GeneBank ID: NZ_PGUZ01000040.1) |
| SEQ ID NO: 17 | Humanized DiC2c1 coding sequence from Desulfovibrio inopinatus DSM |
| | 10711 (GeneBank: NZ_KE386879.1) |
| SEQ ID NO: 18 | Humanized LsC2c1 coding sequence from Laceyella sediminis strain RHA1 |
| | (GeneBank ID: NZ_PVTZ01000002.1) |
| SEQ ID NO: 19 | Humanized SbC2c1 coding sequence from Spirochaetes bacterium |
| | GWB1_27_13 (GeneBank ID: MIAN01000063.1) |
| SEQ ID NO: 20 | Humanized TcC2cl coding sequence from Tuberibacillus calidus DSM |
| | 17572 (GeneBank ID: NZ_KE387196.1) |
| SEQ ID NO: 21 | pCAG-2AeGFP partial sequence (CAG- NLS- Xmal- NheI- NLS- T2A- |
| | eGFP- SV40) |
| SEQ ID NO: 22 | pUC19-U6 partial sequence (U6-BasI-HindIII) |
| SEQ ID NO: 23 | pUC19-U6-AasgRNA partial sequence (U6-AasgRNA_scaffold- |
| | BasI-BasI-terminator) |
| SEQ ID NO: 24 | pUC19-U6-AksgRNA partial sequence (U6-AksgRNA1_scaffold- |
| | BasI-BasI-terminator) |
| SEQ ID NO: 25 | pUC19-U6-AmsgRNA partial sequence (U6-AmsgRNA1_scaffold- |
| | BasI-BasI- terminator) |
| SEQ ID NO: 26 | pUC19-U6-BhsgRNA partial sequence (U6-BhsgRNA1_scaffold- |
| | BasI-BasI-terminator) |
| SEQ ID NO: 27 | pUC19-U6-BssgRNA partial sequence (U6-BssgRNA1_scaffold- |
| | BasI-BasI-terminator) |
| SEQ ID NO: 28 | pUC19-U6-Bs3sgRNA partial sequence (U6-BssgRNA1_scaffold- |
| | BasI-BasI-terminator) |
| SEQ ID NO: 29 | pUC19-U6-LssgRNA partial sequence (U6-BssgRNA1_scaffold- |
| | BasI-BasI-terminator) |
| SEQ ID NO: 30 | pUC19-U6-SbsgRNA partial sequence (U6-BssgRNA1_scaffold- |
| | BasI-BasI-terminator) |
| SEQ ID NO: 31 | AasgRNA_scaffold |
| SEQ ID NO: 32 | AksgRNA1_scaffold |
| SEQ ID NO: 33 | AmsgRNA1_scaffold |
| SEQ ID NO: 34 | BhsgRNA_scaffold |
| SEQ ID NO: 35 | BssgRNA_scaffold |
| SEQ ID NO: 36 | Bs3sgRNA_scaffold |
| SEQ ID NO: 37 | LssgRNA_scaffold |
| SEQ ID NO: 38 | SbsgRNA_scaffold |
| SEQ ID NO: 39 | artsgRA1 scaffold |
| SEQ ID NO: 40 | artsgRA2 scaffold |
| SEQ ID NO: 41 | artsgRA3 scaffold |
| SEQ ID NO: 42 | artsgRA4 scaffold |
| SEQ ID NO: 43 | artsgRA5 scaffold |
| SEQ ID NO: 44 | artsgRA6 scaffold |
| SEQ ID NO: 45 | artsgRA7 scaffold |
| SEQ ID NO: 46 | artsgRA8 scaffold |
| SEQ ID NO: 47 | artsgRA9 scaffold |
| SEQ ID NO: 48 | artsgRA10 scaffold |
| SEQ ID NO: 49 | artsgRA11 scaffold |
| SEQ ID NO: 50 | artsgRA12 scaffold |
| SEQ ID NO: 51 | artsgRA13 scaffold |
| SEQ ID NO: 52 | artsgRA14 scaffold |
| SEQ ID NO: 53 | artsgRA15 scaffold |
| SEQ ID NO: 54 | artsgRA16 scaffold |
| SEQ ID NO: 55 | artsgRA17 scaffold |
| SEQ ID NO: 56 | artsgRA18 scaffold |
| SEQ ID NO: 57 | artsgRA19 scaffold |
| SEQ ID NO: 58 | artsgRA20 scaffold |
| SEQ ID NO: 59 | artsgRA21 scaffold |
| SEQ ID NO: 60 | artsgRA22 scaffold |
| SEQ ID NO: 61 | artsgRA23 scaffold |
| SEQ ID NO: 62 | artsgRA24 scaffold |
| SEQ ID NO: 63 | artsgRA25 scaffold |
| SEQ ID NO: 64 | artsgRA26 scaffold |
| SEQ ID NO: 65 | artsgRA27 scaffold |
| SEQ ID NO: 66 | artsgRA28 scaffold |
| SEQ ID NO: 67 | artsgRA29 scaffold |
| SEQ ID NO: 68 | artsgRA30 scaffold |
| SEQ ID NO: 69 | artsgRA31 scaffold |
| SEQ ID NO: 70 | artsgRA32 scaffold |
| SEQ ID NO: 71 | artsgRA33 scaffold |
| SEQ ID NO: 72 | artsgRA34 scaffold |
| SEQ ID NO: 73 | artsgRA35 scaffold |
| SEQ ID NO: 74 | artsgRA36 scaffold |
| SEQ ID NO: 75 | artsgRA37 scaffold |
| SEQ ID NO: 76 | CCR5 site 10 |
| SEQ ID NO: 77 | CCR5 site 28 |
| SEQ ID NO: 78 | DNMT1 site 16 |
| SEQ ID NO: 79 | RNF2 site 8 |
| SEQ ID NO: 80 | Site 5 |

1. A genome editing system for site-directed modification of a target sequence in the genome of a cell, comprising at least one of the following i) to v):
i) a C2c1 protein or variant thereof, and a guide RNA;
ii) an expression construct comprising a nucleotide sequence encoding a C2c1 protein or variant thereof, and a guide RNA;
iii) a C2c1 protein or variant thereof, and an expression construct comprising a nucleotide sequence encoding a guide RNA;
iv) an expression construct comprising a nucleotide sequence encoding a C2c1 protein or variant thereof, and an expression construct comprising a nucleotide sequence encoding a guide RNA;
v) an expression construct comprising a nucleotide sequence encoding a C2c1 protein or variant thereof and a nucleotide sequence encoding a guide RNA;
wherein the guide RNA is capable of forming complex with the C2c1 protein or variant thereof and targeting the C2c1 protein ortholog or variant thereof to the target sequence in the cell genome.

2. The system of claim 1, wherein the C2c1 protein is AaC2c1 protein derived from Alicyclobacillus acidiphilus, AkC2c1 protein derived from Alicyclobacillus kakegawensis, AmC2c1 protein derived from Alicyclobacillus macrosporangiidus, BhC2c1 protein derived from Bacillus hisashii, BsC2c1 protein derived from the genus Bacillus, Bs3C2c1 protein derived from the genus Bacillus, DiC2c1 protein derived from Desulfovibrio inopinatus, LsC2c1 protein derived from Laceyella sediminis, SbC2c1 protein derived from Spirochaetes bacterium, or TcC2c1 protein derived from Tuberibacillus calidus.

3. The system of claim 1, wherein the C2c1 protein is the AaC2c1 protein derived from Alicyclobacillus acidiphilus NBRC 100859, the AkC2c1 protein derived from Alicyclobacillus kakegawensis NBRC 103104, the AmC2c1 protein derived from Alicyclobacillus macrosporangiidus strain DSM 17980, the BhC2c1 protein derived from Bacillus hisashii strain C4, the BsC2c1 protein derived from genus Bacillus NSP2.1, the Bs3C2c1 protein derived from genus Bacillus V3-13 contig_40, the DiC2c1 protein derived from Desulfovibrio inopinatus DSM 10711, the LsC2c1 protein derived from Laceyella sediminis strain RHA1, the SbC2c1 protein derived from Spirochaetes bacterium GWB1_27_13, or the TcC2c1 protein derived from Tuberibacillus calidus DSM 17572.

4. The system of claim 1, wherein the C2c1 protein comprises the amino acid sequence set forth in any one of SEQ ID NOs: 1-10, or the variant of the C2c1 protein comprises amino acid sequence having at least 80%, at least 85%, at least 90%, at least 95%, at least 96%, at least 97%, at least 98%, at least 99% sequence identity with the amino acid sequence set forth in any one of SEQ ID NOs: 1-10.

5. The system of claim 1, wherein the guide RNA is an sgRNA.

6. The system of claim 5, the sgRNA comprises an sgRNA scaffold sequence encoded by the nucleotide sequence selected from SEQ ID NOs: 31-38 or 39-75.

7. The system of claim 1, wherein the C2c1 protein is a C2c1 protein whose natural locus does not have a CRISPR array.

8. The system of claim 1, wherein the nucleotide sequence encoding a C2c1 protein or variant thereof is codon optimized.

9. The system of claim 8, wherein the nucleotide sequence encoding the C2c1 protein or variant thereof is selected from SEQ ID NOs: 11-20.

10. A method of site-directed modifying a target sequence in the genome of a cell, comprising introducing the system of claim 1 into the cell.

11. The method of claim 10, wherein the cell is derived from mammals such as human, mouse, rat, monkey, dog, pig, sheep, cattle, cat; poultry such as chicken, duck, goose; plants including monocots and dicots, such as rice, corn, wheat, sorghum, barley, soybean, peanut and Arabidopsis thaliana and so on.

12. The method of claim 10, wherein the system is introduced into the cell by a method selected from calcium phosphate transfection, protoplast fusion, electroporation, lipofection, microinjection, viral infection (e.g., baculovirus, vaccinia virus, adenovirus, adeno-associated virus, lentivirus and other viruses), gene gun method, PEG-mediated protoplast transformation, Agrobacterium-mediated transformation.

13. A method of treating a disease in a subject in need thereof, comprising delivering to the subject an effective amount of the genome editing system of claim 1 to modify a gene related to the disease in the subject.

14. Use of the genome editing system of claim 1 for the preparation of a pharmaceutical composition for treating a disease in a subject in need thereof, wherein the genome editing system is for modifying a gene related to the disease in the subject.

15. A pharmaceutical composition for treating a disease in a subject in need thereof, comprising the genome editing system of claim 1 and a pharmaceutically acceptable carrier, wherein the genome editing system is for modifying a gene related to the disease in the subject.

16. The method of claim 13, wherein the subject is a mammal, optionally a human.

17. The method of claim 16, wherein the disease is selected from tumors, inflammation, Parkinson's disease, cardiovascular disease, Alzheimer's disease, autism, drug addiction, age-related macular degeneration, schizophrenia, and hereditary diseases.


SEQUENCE LISTING (83 sequences)

SEQ ID NO: 1 - protein, 1129 aa, Unknown
  Alicyclobacillus acidiphilus NBRC 100859
MAVKSMKVKLRLDNMPEIRAGLWKLHTEVNAGVRYYTEWLSLLRQENLYRRSPNGDGEQECYKTAEECKAELLERLRARQVENGHCGPAGSDDELLQLARQLYELLVPQAIGAKGDAQQIARKFLSPLADKDAVGGLGIAKAGNKPRWVRMREAGEPGWEEEKAKAEARKSTDRTADVLRALADFGLKPLMRVYTDSDMSSVQWKPLRKGQAVRTWDRDMFQQAIERMMSWESWNQRVGEAYAKLVEQKSRFEQKNFVGQEHLVQLVNQLQQDMKEASHGLESKEQTAHYLTGRALRGSDKVFEKWEKLDPDAPFDLYDTEIKNVQRRNTRRFGSHDLFAKLAEPKYQALWREDASFLTRYAVYNSIVRKLNHAKMFATFTLPDATAHPIWTRFDKLGGNLHQYTFLFNEFGEGRHAIRFQKLLTVEDGVAKEVDDVTVPISMSAQLDDLLPRDPHELVALYFQDYGAEQHLAGEFGGAKIQYRRDQLNHLHARRGARDVYLNLSVRVQSQSEARGERRPPYAAVFRLVGDNHRAFVHFDKLSDYLAEHPDDGKLGSEGLLSGLRVMSVDLGLRTSASISVFRVARKDELKPNSEGRVPFCFPIEGNENLVAVHERSQLLKLPGETESKDLRAIREERQRTLRQLRTQLAYLRLLVRCGSEDVGRRERSWAKLIEQPMDANQMTPDWREAFEDELQKLKSLYGICGDREWTEAVYESVRRVWRHMGKQVRDWRKDVRSGERPKIRGYQKDVVGGNSIEQIEYLERQYKFLKSWSFFGKVSGQVIRAEKGSRFAITLREHIDHAKEDRLKKLADRIIMEALGYVYALDDERGKGKWVAKYPPCQLILLEELSEYQFNNDRPPSENNQLMQWSHRGVFQELLNQAQVHDLLVGTMYAAFSSRFDARTGAPGIRCRRVPARCAREQNPEPFPWWLNKFVAEHKLDGCPLRADDLIPTGEGEFFVSPFSAEEGDFHQIHADLNAAQNLQRRLWSDFDISQIRLRCDWGEVDGEPVLIPRTTGKRTADSYGNKVFYTKTGVTYYERERGKKRRKVFAQEELSEEEAELLVEADEAREKSVVLMRDPSGIINRGDWTRQKEFWSMVNQRIEGYLVKQIRSRVRLQESACENTGDI

SEQ ID NO: 2 - protein, 1147 aa, Unknown
  Alicyclobacillus kakegawensis NBRC 103104
MAVKSIKVKLRLSECPDILAGMWQLHRATNAGVRYYTEWVSLMRQEILYSRGPDGGQQCYMTAEDCQRELLRRLRNRQLHNGRQDQPGTDADLLAISRRLYEILVLQSIGKRGDAQQIASSFLSPLVDPNSKGGRGEAKSGRKPAWQKMRDQGDPRWVAAREKYEQRKAVDPSKEILNSLDALGLRPLFAVFTETYRSGVDWKPLGKSQGVRTWDRDMFQQALERLMSWESWNRRVGEEYARLFQQKMKFEQEHFAEQSHLVKLARALEADMRAASQGFEAKRGTAHQITRRALRGADRVFEIWKSIPEEALFSQYDEVIRQVQAEKRRDFGSHDLFAKLAEPKYQPLWRADETFLTRYALYNGVLRDLEKARQFATFTLPDACVNPIWTRFESSQGSNLHKYEFLFDHLGPGRHAVRFQRLLVVESEGAKERDSVVVPVAPSGQLDKLVLREEEKSSVALHLHDTARPDGFMAEWAGAKLQYERSTLARKARRDKQGMRSWRRQPSMLMSAAQMLEDAKQAGDVYLNISVRVKSPSEVRGQRRPPYAALFRIDDKQRRVTVNYNKLSAYLEEHPDKQIPGAPGLLSGLRVMSVDLGLRTSASISVFRVAKKEEVEALGDGRPPHYYPIHGTDDLVAVHERSHLIQMPGETETKQLRKLREERQAVLRPLFAQLALLRLLVRCGAADERIRTRSWQRLTKQGREFTKRLTPSWREALELELTRLEAYCGRVPDDEWSRIVDRTVIALWRRMGKQVRDWRKQVKSGAKVKVKGYQLDVVGGNSLAQIDYLEQQYKFLRRWSFFARASGLVVRADRESHFAVALRQHIENAKRDRLKKLADRILMEALGYVYEASGPREGQWTAQHPPCQLIILEELSAYRFSDDRPPSENSKLMAWGHRGILEELVNQAQVHDVLVGTVYAAFSSRFDARTGAPGVRCRRVPARFVGATVDDSLPLWLTEFLDKHRLDKNLLRPDDVIPTGEGEFLVSPCGEEAARVRQVHADINAAQNLQRRLWQNFDITELRLRCDVKMGGEGTVLVPRVNNARAKQLFGKKVLVSQDGVTFFERSQTGGKPHSEKQTDLTDKELELIAEADEARAKSVVLFRDPSGHIGKGHWIRQREFWSLVKQRIESHTAERIRVRGVGSSLD

SEQ ID NO: 3 - protein, 1146 aa, Unknown
  Alicyclobacillus macrosporangiidus strain DSM 17980
MNVAVKSIKVKLMLGHLPEIREGLWHLHEAVNLGVRYYTEWLALLRQGNLYRRGKDGAQECYMTAEQCRQELLVRLRDRQKRNGHTGDPGTDEELLGVARRLYELLVPQSVGKKGQAQMLASGFLSPLADPKSEGGKGTSKSGRKPAWMGMKEAGDSRWVEAKARYEANKAKDPTKQVIASLEMYGLRPLFDVFTETYKTIRWMPLGKHQGVRAWDRDMFQQSLERLMSWESWNERVGAEFARLVDRRDRFREKHFTGQEHLVALAQRLEQEMKEASPGFESKSSQAHRITKRALRGADGIIDDWLKLSEGEPVDRFDEILRKRQAQNPRRFGSHDLFLKLAEPVFQPLWREDPSFLSRWASYNEVLNKLEDAKQFATFTLPSPCSNPVWARFENAEGTNIFKYDFLFDHFGKGRHGVRFQRMIVMRDGVPTEVEGIVVPIAPSRQLDALAPNDAASPIDVFVGDPAAPGAFRGQFGGAKIQYRRSALVRKGRREEKAYLCGFRLPSQRRTGTPADDAGEVFLNLSLRVESQSEQAGRRNPPYAAVFHISDQTRRVIVRYGEIERYLAEHPDTGIPGSRGLTSGLRVMSVDLGLRTSAAISVFRVAHRDELTPDAHGRQPFFFPIHGMDHLVALHERSHLIRLPGETESKKVRSIREQRLDRLNRLRSQMASLRLLVRTGVLDEQKRDRNWERLQSSMERGGERMPSDWWDLFQAQVRYLAQHRDASGEAWGRMVQAAVRTLWRQLAKQVRDWRKEVRRNADKVKIRGIARDVPGGHSLAQLDYLERQYRFLRSWSAFSVQAGQVVRAERDSRFAVALREHIDNGKKDRLKKLADRILMEALGYVYVTDGRRAGQWQAVYPPCQLVLLEELSEYRFSNDRPPSENSQLMVWSHRGVLEELIHQAQVHDVLVGTIPAAFSSRFDARTGAPGIRCRRVPSIPLKDAPSIPIWLSHYLKQTERDAAALRPGELIPTGDGEFLVTPAGRGASGVRVVHADINAAHNLQRRLWENFDLSDIRVRCDRREGKDGTVVLIPRLTNQRVKERYSGVIFTSEDGVSFTVGDAKTRRRSSASQGEGDDLSDEEQELLAEADDARERSVVLFRDPSGFVNGGRWTAQRAFWGMVHNRIETLLAERFSVSGAAEKVRG

SEQ ID NO: 4 - protein, 1108 aa, Unknown
  Bacillus hisashii strain C4
MATRSFILKIEPNEEVKKGLWKTHEVLNHGIAYYMNILKLIRQEAIYEHHEQDPKNPKKVSKAEIQAELWDFVLKMQKCNSFTHEVDKDEVFNILRELYEELVPSSVEKKGEANQLSNKFLYPLVDPNSQSGKGTASSGRKPRWYNLKIAGDPSWEEEKKKWEEDKKKDPLAKILGKLAEYGLIPLFIPYTDSNEPIVKEIKWMEKSRNQSVRRLDKDMFIQALERFLSWESWNLKVKEEYEKVEKEYKTLEERIKEDIQALKALEQYEKERQEQLLRDTLNTNEYRLSKRGLRGWREIIQKWLKMDENEPSEKYLEVFKDYQRKHPREAGDYSVYEFLSKKENHFIWRNHPEYPYLYATFCEIDKKKKDAKQQATFTLADPINHPLWVRFEERSGSNLNKYRILTEQLHTEKLKKKLTVQLDRLIYPTESGGWEEKGKVDIVLLPSRQFYNQIFLDIEEKGKHAFTYKDESIKFPLKGTLGGARVQFDRDHLRRYPHKVESGNVGRIYFNMTVNIEPTESPVSKSLKIHRDDFPKVVNFKPKELTEWIKDSKGKKLKSGIESLEIGLRVMSIDLGQRQAAAASIFEVVDQKPDIEGKLFFPIKGTELYAVHRASFNIKLPGETLVKSREVLRKAREDNLKLMNQKLNFLRNVLHFQQFEDITEREKRVTKWISRQENSDVPLVYQDELIQIRELMYKPYKDWVAFLKQLHKRLEVEIGKEVKHWRKSLSDGRKGLYGISLKNIDEIDRTRKFLLRWSLRPTEPGEVRRLEPGQRFAIDQLNHLNALKEDRLKKMANTIIMHALGYCYDVRKKKWQAKNPACQIILFEDLSNYNPYEERSRFENSKLMKWSRREIPRQVALQGEIYGLQVGEVGAQFSSRFHAKTGSPGIRCSVVTKEKLQDNRFFKNLQREGRLTLDKIAVLKEGDLYPDKGGEKFISLSKDRKCVTTHADINAAQNLQKRFWTRTHGFYKVYCKAYQVDGQTVYIPESKDQKQKIIEEFGEGYFILKDGVYEWVNAGKLKIKKGSSKQSSSELVDSDILKDSFDLASELKGEKLMLYRDPSGNVFPSDKWMAAGVFFGKLERILISKLTNQYSISTIEDDSSKQSM

SEQ ID NO: 5 - protein, 1108 aa, Unknown
  Bacillus sp. NSP2.1
MAIRSIKLKLKTHTGPEAQNLRKGIWRTHRLLNEGVAYYMKMLLLFRQESTGERPKEELQEELICHIREQQQRNQADKNTQALPLDKALEALRQLYELLVPSSVGQSGDAQIISRKFLSPLVDPNSEGGKGTSKAGAKPTWQKKKEANDPTWEQDYEKWKKRREEDPTASVITTLEEYGIRPIFPLYTNTVTDIAWLPLQSNQFVRTWDRDMLQQAIERLLSWESWNKRVQEEYAKLKEKMAQLNEQLEGGQEWISLLEQYEENRERELRENMTAANDKYRITKRQMKGWNELYELWSTFPASASHEQYKEALKRVQQRLRGRFGDAHFFQYLMEEKNRLIWKGNPQRIHYFVARNELTKRLEEAKQSATMTLPNARKHPLWVRFDARGGNLQDYYLTAEADKPRSRRFVTFSQLIWPSESGWMEKKDVEVELALSRQFYQQVKLLKNDKGKQKIEFKDKGSGSTFNGHLGGAKLQLERGDLEKEEKNFEDGEIGSVYLNVVIDFEPLQEVKNGRVQAPYGQVLQLIRRPNEFPKVTTYKSEQLVEWIKASPQHSAGVESLASGFRVMSIDLGLRAAAATSIFSVEESSDKNAADFSYWIEGTPLVAVHQRSYMLRLPGEQVEKQVMEKRDERFQLHQRVKFQIRVLAQIMRMANKQYGDRWDELDSLKQAVEQKKSPLDQTDRTFWEGIVCDLTKVLPRNEADWEQAVVQIHRKAEEYVGKAVQAWRKRFAADERKGIAGLSMWNIEELEGLRKLLISWSRRTRNPQEVNRFERGHTSHQRLLTHIQNVKEDRLKQLSHAIVMTALGYVYDERKQEWCAEYPACQVILFENLSQYRSNLDRSTKENSTLMKWAHRSIPKYVHMQAEPYGIQIGDVRAEYSSRFYAKTGTPGIRCKKVRGQDLQGRRFENLQKRLVNEQFLTEEQVKQLRPGDIVPDDSGELFMTLTDGSGSKEVVFLQADINAAHNLQKRFWQRYNELFKVSCRVIVRDEEEYLVPKTKSVQAKLGKGLFVKKSDTAWKDVYVWDSQAKLKGKTTFTEESESPEQLEDFQEIIEEAEEAKGTYRTLFRDPSGVFFPESVWYPQKDFWGEVKRKLYGKLRERFLTKAR

SEQ ID NO: 6 - protein, 1112 aa, Unknown
  Bacillus sp. V3-13
MAIRSIKLKMKTNSGTDSIYLRKALWRTHQLINEGIAYYMNLLTLYRQEAIGDKTKEAYQAELINIIRNQQRNNGSSEEHGSDQEILALLRQLYELIIPSSIGESGDANQLGNKFLYPLVDPNSQSGKGTSNAGRKPRWKRLKEEGNPDWELEKKKDEERKAKDPTVKIFDNLNKYGLLPLFPLFTNIQKDIEWLPLGKRQSVRKWDKDMFIQAIERLLSWESWNRRVADEYKQLKEKTESYYKEHLTGGEEWIEKIRKFEKERNMELEKNAFAPNDGYFITSRQIRGWDRVYEKWSKLPESASPEELWKVVAEQQNKMSEGFGDPKVFSFLANRENRDIWRGHSERIYHIAAYNGLQKKLSRTKEQATFTLPDAIEHPLWIRYESPGGTNLNLFKLEEKQKKNYYVTLSKIIWPSEEKWIEKENIEIPLAPSIQFNRQIKLKQHVKGKQEISFSDYSSRISLDGVLGGSRIQFNRKYIKNHKELLGEGDIGPVFFNLVVDVAPLQETRNGRLQSPIGKALKVISSDFSKVIDYKPKELMDWMNTGSASNSFGVASLLEGMRVMSIDMGQRTSASVSIFEVVKELPKDQEQKLFYSINDTELFAIHKRSFLLNLPGEVVTKNNKQQRQERRKKRQFVRSQIRMLANVLRLETKKTPDERKKAIHKLMEIVQSYDSWTASQKEVWEKELNLLTNMAAFNDEIWKESLVELHHRIEPYVGQIVSKWRKGLSEGRKNLAGISMWNIDELEDTRRLLISWSKRSRTPGEANRIETDEPFGSSLLQHIQNVKDDRLKQMANLIIMTALGFKYDKEEKDRYKRWKETYPACQIILFENLNRYLFNLDRSRRENSRLMKWAHRSIPRTVSMQGEMFGLQVGDVRSEYSSRFHAKTGAPGIRCHALTEEDLKAGSNTLKRLIEDGFINESELAYLKKGDIIPSQGGELFVTLSKRYKKDSDNNELTVIHADINAAQNLQKRFWQQNSEVYRVPCQLARMGEDKLYIPKSQTETIKKYFGKGSFVKNNTEQEVYKWEKSEKMKIKTDTTFDLQDLDGFEDISKTIELAQEQQKKYLTMFRDPSGYFFNNETWRPQKEYWSIVNNIIKSCLKKKILSNKVEL

SEQ ID NO: 7 - protein, 1149 aa, Unknown
  Desulfovibrio inopinatus DSM 10711
MPTRTINLKLVLGKNPENATLRRALFSTHRLVNQATKRIEEFLLLCRGEAYRTVDNEGKEAEIPRHAVQEEALAFAKAAQRHNGCISTYEDQEILDVLRQLYERLVPSVNENNEAGDAQAANAWVSPLMSAESEGGLSVYDKVLDPPPVWMKLKEEKAPGWEAASQIWIQSDEGQSLLNKPGSPPRWIRKLRSGQPWQDDFVSDQKKKQDELTKGNAPLIKQLKEMGLLPLVNPFFRHLLDPEGKGVSPWDRLAVRAAVAHFISWESWNHRTRAEYNSLKLRRDEFEAASDEFKDDFTLLRQYEAKRHSTLKSIALADDSNPYRIGVRSLRAWNRVREEWIDKGATEEQRVTILSKLQTQLRGKFGDPDLFNWLAQDRHVHLWSPRDSVTPLVRINAVDKVLRRRKPYALMTFAHPRFHPRWILYEAPGGSNLRQYALDCTENALHITLPLLVDDAHGTWIEKKIRVPLAPSGQIQDLTLEKLEKKKNRLYYRSGFQQFAGLAGGAEVLFHRPYMEHDERSEESLLERPGAVWFKLTLDVATQAPPNWLDGKGRVRTPPEVHHFKTALSNKSKHTRTLQPGLRVLSVDLGMRTFASCSVFELIEGKPETGRAFPVADERSMDSPNKLWAKHERSFKLTLPGETPSRKEEEERSIARAEIYALKRDIQRLKSLLRLGEEDNDNRRDALLEQFFKGWGEEDVVPGQAFPRSLFQGLGAAPFRSTPELWRQHCQTYYDKAEACLAKHISDWRKRTRPRPTSREMWYKTRSYHGGKSIWMLEYLDAVRKLLLSWSLRGRTYGAINRQDTARFGSLASRLLHHINSLKEDRIKTGADSIVQAARGYIPLPHGKGWEQRYEPCQLILFEDLARYRFRVDRPRRENSQLMQWNHRAIVAETTMQAELYGQIVENTAAGFSSRFHAATGAPGVRCRFLLERDFDNDLPKPYLLRELSWMLGNTKVESEEEKLRLLSEKIRPGSLVPWDGGEQFATLHPKRQTLCVIHADMNAAQNLQRRFFGRCGEAFRLVCQPHGDDVLRLASTPGARLLGALQQLENGQGAFELVRDMGSTSQMNRFVMKSLGKKKIKPLQDNNGDDELEDVLSVLPEEDDTGRITVFRDSSGIFFPCNVWIPAKQFWPAVRAMIWKVMASHSLG

SEQ ID NO: 8 - protein, 1090 aa, Unknown
  Laceyella sediminis strain RHA1
MSIRSFKLKIKTKSGVNAEELRRGLWRTHQLINDGIAYYMNWLVLLRQEDLFIRNEETNEIEKRSKEEIQGELLERVHKQQQRNQWSGEVDDQTLLQTLRHLYEEIVPSVIGKSGNASLKARFFLGPLVDPNNKTTKDVSKSGPTPKWKKMKDAGDPNWVQEYEKYMAERQTLVRLEEMGLIPLFPMYTDEVGDIHWLPQASGYTRTWDRDMFQQAIERLLSWESWNRRVRERRAQFEKKTHDFASRFSESDVQWMNKLREYEAQQEKSLEENAFAPNEPYALTKKALRGWERVYHSWMRLDSAASEEAYWQEVATCQTAMRGEFGDPAIYQFLAQKENHDIWRGYPERVIDFAELNHLQRELRRAKEDATFTLPDSVDHPLWVRYEAPGGTNIHGYDLVQDTKRNLTLILDKFILPDENGSWHEVKKVPFSLAKSKQFHRQVWLQEEQKQKKREVVFYDYSTNLPHLGTLAGAKLQWDRNFLNKRTQQQIEETGEIGKVFFNISVDVRPAVEVKNGRLQNGLGKALTVLTHPDGTKIVTGWKAEQLEKWVGESGRVSSLGLDSLSEGLRVMSIDLGQRTSATVSVFEITKEAPDNPYKFFYQLEGTELFAVHQRSFLLALPGENPPQKIKQMREIRWKERNRIKQQVDQLSAILRLHKKVNEDERIQAIDKLLQKVASWQLNEEIATAWNQALSQLYSKAKENDLQWNQAIKNAHHQLEPVVGKQISLWRKDLSTGRQGIAGLSLWSIEELEATKKLLTRWSKRSREPGVVKRIERFETFAKQIQHHINQVKENRLKQLANLIVMTALGYKYDQEQKKWIEVYPACQVVLFENLRSYRFSYERSRRENKKLMEWSHRSIPKLVQMQGELFGLQVADVYAAYSSRYHGRTGAPGIRCHALTEADLRNETNIIHELIEAGFIKEEHRPYLQQGDLVPWSGGELFATLQKPYDNPRILTLHADINAAQNIQKRFWHPSMWFRVNCESVMEGEIVTYVPKNKTVHKKQGKTFRFVKVEGSDVYEWAKWSKNRNKNTFSSITERKPPSSMILFRDPSGTFFKEQEWVEQKTFWGKVQSMIQAYMKKTIVQRMEE

SEQ ID NO: 9 - protein, 1119 aa, Unknown
  Spirochaetes bacterium GWB1_27_13
MSFTISYPFKLIIKNKDEAKALLDTHQYMNEGVKYYLEKLLMFRQEKIFIGEDETGKRIYIEETEYKKQIEEFYLIKKTELGRNLTLTLDEFKTLMRELYICLVSSSMENKKGFPNAQQASLNIFSPLFDAESKGYILKEENNNISLIHKDYGKILLKRLRDNNLIPIFTKFTDIKKITAKLSPTALDRMIFAQAIEKLLSYESWCKLMIKERFDKEVKIKELENKCENKQERDKIFEILEKYEEERQKTFEQDSGFAKKGKFYITGRMLKGFDEIKEKWLKEKDRSEQNLINILNKYQTDNSKLVGDRNLFEFIIKLENQCLWNGDIDYLKIKRDINKNQIWLDRPEMPRFTMPDFKKHPLWYRYEDPSNSNFRNYKIEVVKDENYITIPLITERNNEYFEENYTFNLAKLKKLSENITFIPKSKNKEFEFIDSNDEEEDKKDQKKSKQYIKYCDTAKNTSYGKSGGIRLYFNRNELENYKDGKKMDSYTVFTLSIRDYKSLFAKEKLQPQIFNTVDNKITSLKIQKKFGNEEQTNFLSYFTQNQITKKDWMDEKTFQNVKELNEGIRVLSVDLGQRFFAAVSCFEIMSEIDNNKLFFNLNDQNHKIIRINDKNYYAKHIYSKTIKLSGEDDDLYKERKINKNYKLSYQERKNKIGIFTRQINKLNQLLKIIRNDEIDKEKFKELIETTKRYVKNTYNDGIIDWNNVDNKILSYENKEDVINLHKELDKKLEIDFKEFIRECRKPIFRSGGLSMQRIDFLEKLNKLKRKWVARTQKSAESIVLTPKFGYKLKEHINELKDNRVKQGVNYILMTALGYIKDNEIKNDSKKKQKEDWVKKNRACQIILMEKLTEYTFAEDRPREENSKLRMWSHRQIFNFLQQKASLWGILVGDVFAPYTSKCLSDNNAPGIRCHQVTKKDLIDNSWFLKIVVKDDAFCDLIEINKENVKNKSIKINDILPLRGGELFASIKDGKLHIVQADINASRNIAKRFLSQINPFRVVLKKDKDETFHLKNEPNYLKNYYSILNFVPTNEELTFFKVEENKDIKPTKRIKMDKHEKESTDEGDDYSKNQIALFRDDSGIFFDKSLWVDGKIFWSVVKNKMTKLLRERNNKKNGSK

SEQ ID NO: 10 - protein, 1142 aa, Unknown
  Tuberibacillus calidus DSM 17572
MNIHLKELIRMATKSFILKMKTKNNPQLRLSLWKTHELFNFGVAYYMDLLSLFRQKDLYMHNDEDPDHPVVLKKEEIQERLWMKVRETQQKNGFHGEVSKDEVLETLRALYEELVPSAVGKSGEANQISNKYLYPLTDPASQSGKGTANSGRKPRWKKLKEAGDPSWKDAYEKWEKERQEDPKLKILAALQSFGLIPLFRPFTENDHKAVISVKWMPKSKNQSVRKFDKDMFNQAIERFLSWESWNEKVAEDYEKTVSIYESLQKELKGISTKAFEIMERVEKAYEAHLREITFSNSTYRIGNRAIRGWTEIVKKWMKLDPSAPQGNYLDVVKDYQRRHPRESGDFKLFELLSRPENQAAWREYPEFLPLYVKYRHAEQRMKTAKKQATFTLCDPIRHPLWVRYEERSGTNLNKYRLIMNEKEKVVQFDRLICLNADGHYEEQEDVTVPLAPSQQFDDQIKFSSEDTGKGKHNFSYYHKGINYELKGTLGGARIQFDREHLLRRQGVKAGNVGRIFLNVTLNIEPMQPFSRSGNLQTSVGKALKVYVDGYPKVVNFKPKELTEHIKESEKNTLTLGVESLPTGLRVMSVDLGQRQAAAISIFEVVSEKPDDNKLFYPVKDTDLFAVHRTSFNIKLPGEKRTERRMLEQQKRDQAIRDLSRKLKFLKNVLNMQKLEKTDEREKRVNRWIKDREREEENPVYVQEFEMISKVLYSPHSVWVDQLKSIHRKLEEQLGKEISKWRQSISQGRQGVYGISLKNIEDIEKTRRLLFRWSMRPENPGEVKQLQPGERFAIDQQNHLNHLKDDRIKKLANQIVMTALGYRYDGKRKKWIAKHPACQLVLFEDLSRYAFYDERSRLENRNLMRWSRREIPKQVAQIGGLYGLLVGEVGAQYSSRFHAKSGAPGIRCRVVKEHELYITEGGQKVRNQKFLDSLVENNIIEPDDARRLEPGDLIRDQGGDKFATLDERGELVITHADINAAQNLQKRFWTRTHGLYRIRCESREIKDAVVLVPSDKDQKEKMENLFGIGYLQPFKQENDVYKWVKGEKIKGKKTSSQSDDKELVSEILQEASVMADELKGNRKTLFRDPSGYVFPKDRWYTGGRYFGTLEHLLKRKLAERRLFDGGSSRRGLFNGTDSNTNVE

SEQ ID NO: 11 - DNA, 3387 bp, Unknown
  Alicyclobacillus acidiphilus NBRC 100859
atggccgtgaagagcatgaaggtgaagctgcgcctggacaacatgcccgagatccgcgccggcctgtggaagctgcacaccgaggtgaacgccggcgtgcgctactacaccgagtggctgagcctgctgcgccaggagaacctgtaccgccgcagccccaacggcgacggcgagcaggagtgctacaagaccgccgaggagtgcaaggccgagctgctggagcgcctgcgcgcccgccaggtggagaacggccactgcggccccgccggcagcgacgacgagctgctgcagctggcccgccagctgtacgagctgctggtgccccaggccatcggcgccaagggcgacgcccagcagatcgcccgcaagttcctgagccccctggccgacaaggacgccgtgggcggcctgggcatcgccaaggccggcaacaagccccgctgggtgcgcatgcgcgaggccggcgagcccggctgggaggaggagaaggccaaggccgaggcccgcaagagcaccgaccgcaccgccgacgtgctgcgcgccctggccgacttcggcctgaagcccctgatgcgcgtgtacaccgacagcgacatgagcagcgtgcagtggaagcccctgcgcaagggccaggccgtgcgcacctgggaccgcgacatgttccagcaggccatcgagcgcatgatgagctgggagagctggaaccagcgcgtgggcgaggcctacgccaagctggtggagcagaagagccgcttcgagcagaagaacttcgtgggccaggagcacctggtgcagctggtgaaccagctgcagcaggacatgaaggaggccagccacggcctggagagcaaggagcagaccgcccactacctgaccggccgcgccctgcgcggcagcgacaaggtgttcgagaagtgggagaagctggaccccgacgcccccttcgacctgtacgacaccgagatcaagaacgtgcagcgccgcaacacccgccgcttcggcagccacgacctgttcgccaagctggccgagcccaagtaccaggccctgtggcgcgaggacgccagcttcctgacccgctacgccgtgtacaacagcatcgtgcgcaagctgaaccacgccaagatgttcgccaccttcaccctgcccgacgccaccgcccaccccatctggacccgcttcgacaagctgggcggcaacctgcaccagtacaccttcctgttcaacgagttcggcgagggccgccacgccatccgcttccagaagctgctgaccgtggaggacggcgtggccaaggaggtggacgacgtgaccgtgcccatcagcatgagcgcccagctggacgacctgctgccccgcgacccccacgagctggtggccctgtacttccaggactacggcgccgagcagcacctggccggcgagttcggcggcgccaagatccagtaccgccgcgaccagctgaaccacctgcacgcccgccgcggcgcccgcgacgtgtacctgaacctgagcgtgcgcgtgcagagccagagcgaggcccgcggcgagcgccgccccccctacgccgccgtgttccgcctggtgggcgacaaccaccgcgccttcgtgcacttcgacaagctgagcgactacctggccgagcaccccgacgacggcaagctgggcagcgagggcctgctgagcggcctgcgcgtgatgagcgtggacctgggcctgcgcaccagcgccagcatcagcgtgttccgcgtggcccgcaaggacgagctgaagcccaacagcgagggccgcgtgcccttctgcttccccatcgagggcaacgagaacctggtggccgtgcacgagcgcagccagctgctgaagctgcccggcgagaccgagagcaaggacctgcgcgccatccgcgaggagcgccagcgcaccctgcgccagctgcgcacccagctggcctacctgcgcctgctggtgcgctgcggcagcgaggacgtgggccgccgcgagcgcagctgggccaagctgatcgagcagcccatggacgccaaccagatgacccccgactggcgcgaggccttcgaggacgagctgcagaagctgaagagcctgtacggcatctgcggcgaccgcgagtggaccgaggccgtgtacgagagcgtgcgccgcgtgtggcgccacatgggcaagcaggtgcgcgactggcgcaaggacgtgcgcagcggcgagcgccccaagatccgcggctaccagaaggacgtggtgggcggcaacagcatcgagcagatcgagtacctggagcgccagtacaagttcctgaagagctggagcttcttcggcaaggtgagcggccaggtgatccgcgccgagaagggcagccgcttcgccatcaccctgcgcgagcacatcgaccacgccaaggaggaccgcctgaagaagctggccgaccgcatcatcatggaggccctgggctacgtgtacgccctggacgacgagcgcggcaagggcaagtgggtggccaagtaccccccctgccagctgatcctgctggaggagctgagcgagtaccagttcaacaacgaccgcccccccagcgagaacaaccagctgatgcagtggagccaccgcggcgtgttccaggagctgctgaaccaggcccaggtgcacgacctgctggtgggcaccatgtacgccgccttcagcagccgcttcgacgcccgcaccggcgcccccggcatccgctgccgccgcgtgcccgcccgctgcgcccgcgagcagaaccccgagcccttcccctggtggctgaacaagttcgtggccgagcacaagctggacggctgccccctgcgcgccgacgacctgatccccaccggcgagggcgagttcttcgtgagccccttcagcgccgaggagggcgacttccaccagatccacgccgacctgaacgccgcccagaacctgcagcgccgcctgtggagcgacttcgacatcagccagatccgcctgcgctgcgactggggcgaggtggacggcgagcccgtgctgatcccccgcaccaccggcaagcgcaccgccgacagctacggcaacaaggtgttctacaccaagaccggcgtgacctactacgagcgcgagcgcggcaagaagcgccgcaaggtgttcgcccaggaggagctgagcgaggaggaggccgagctgctggtggaggccgacgaggcccgcgagaagagcgtggtgctgatgcgcgaccccagcggcatcatcaaccgcggcgactggacccgccagaaggagttctggagcatggtgaaccagcgcatcgagggctacctggtgaagcagatccgcagccgcgtgcgcctgcaggagagcgcctgcgagaacaccggcgacatc

SEQ ID NO: 12 - DNA, 3441 bp, Unknown
  Alicyclobacillus kakegawensis NBRC 103104
atggccgtgaagagcatcaaggtgaagctgcgcctgagcgagtgccccgacatcctggccggcatgtggcagctgcaccgcgccaccaacgccggcgtgcgctactacaccgagtgggtgagcctgatgcgccaggagatcctgtacagccgcggccccgacggcggccagcagtgctacatgaccgccgaggactgccagcgcgagctgctgcgccgcctgcgcaaccgccagctgcacaacggccgccaggaccagcccggcaccgacgccgacctgctggccatcagccgccgcctgtacgagatcctggtgctgcagagcatcggcaagcgcggcgacgcccagcagatcgccagcagcttcctgagccccctggtggaccccaacagcaagggcggccgcggcgaggccaagagcggccgcaagcccgcctggcagaagatgcgcgaccagggcgacccccgctgggtggccgcccgcgagaagtacgagcagcgcaaggccgtggaccccagcaaggagatcctgaacagcctggacgccctgggcctgcgccccctgttcgccgtgttcaccgagacctaccgcagcggcgtggactggaagcccctgggcaagagccagggcgtgcgcacctgggaccgcgacatgttccagcaggccctggagcgcctgatgagctgggagagctggaaccgccgcgtgggcgaggagtacgcccgcctgttccagcagaagatgaagttcgagcaggagcacttcgccgagcagagccacctggtgaagctggcccgcgccctggaggccgacatgcgcgccgccagccagggcttcgaggccaagcgcggcaccgcccaccagatcacccgccgcgccctgcgcggcgccgaccgcgtgttcgagatctggaagagcatccccgaggaggccctgttcagccagtacgacgaggtgatccgccaggtgcaggccgagaagcgccgcgacttcggcagccacgacctgttcgccaagctggccgagcccaagtaccagcccctgtggcgcgccgacgagaccttcctgacccgctacgccctgtacaacggcgtgctgcgcgacctggagaaggcccgccagttcgccaccttcaccctgcccgacgcctgcgtgaaccccatctggacccgcttcgagagcagccagggcagcaacctgcacaagtacgagttcctgttcgaccacctgggccccggccgccacgccgtgcgcttccagcgcctgctggtggtggagagcgagggcgccaaggagcgcgacagcgtggtggtgcccgtggcccccagcggccagctggacaagctggtgctgcgcgaggaggagaagagcagcgtggccctgcacctgcacgacaccgcccgccccgacggcttcatggccgagtgggccggcgccaagctgcagtacgagcgcagcaccctggcccgcaaggcccgccgcgacaagcagggcatgcgcagctggcgccgccagcccagcatgctgatgagcgccgcccagatgctggaggacgccaagcaggccggcgacgtgtacctgaacatcagcgtgcgcgtgaagagccccagcgaggtgcgcggccagcgccgccccccctacgccgccctgttccgcatcgacgacaagcagcgccgcgtgaccgtgaactacaacaagctgagcgcctacctggaggagcaccccgacaagcagatccccggcgcccccggcctgctgagcggcctgcgcgtgatgagcgtggacctgggcctgcgcaccagcgccagcatcagcgtgttccgcgtggccaagaaggaggaggtggaggccctgggcgacggccgccccccccactactaccccatccacggcaccgacgacctggtggccgtgcacgagcgcagccacctgatccagatgcccggcgagaccgagaccaagcagctgcgcaagctgcgcgaggagcgccaggccgtgctgcgccccctgttcgcccagctggccctgctgcgcctgctggtgcgctgcggcgccgccgacgagcgcatccgcacccgcagctggcagcgcctgaccaagcagggccgcgagttcaccaagcgcctgacccccagctggcgcgaggccctggagctggagctgacccgcctggaggcctactgcggccgcgtgcccgacgacgagtggagccgcatcgtggaccgcaccgtgatcgccctgtggcgccgcatgggcaagcaggtgcgcgactggcgcaagcaggtgaagagcggcgccaaggtgaaggtgaagggctaccagctggacgtggtgggcggcaacagcctggcccagatcgactacctggagcagcagtacaagttcctgcgccgctggagcttcttcgcccgcgccagcggcctggtggtgcgcgccgaccgcgagagccacttcgccgtggccctgcgccagcacatcgagaacgccaagcgcgaccgcctgaagaagctggccgaccgcatcctgatggaggccctgggctacgtgtacgaggccagcggcccccgcgagggccagtggaccgcccagcaccccccctgccagctgatcatcctggaggagctgagcgcctaccgcttcagcgacgaccgcccccccagcgagaacagcaagctgatggcctggggccaccgcggcatcctggaggagctggtgaaccaggcccaggtgcacgacgtgctggtgggcaccgtgtacgccgccttcagcagccgcttcgacgcccgcaccggcgcccccggcgtgcgctgccgccgcgtgcccgcccgcttcgtgggcgccaccgtggacgacagcctgcccctgtggctgaccgagttcctggacaagcaccgcctggacaagaacctgctgcgccccgacgacgtgatccccaccggcgagggcgagttcctggtgagcccctgcggcgaggaggccgcccgcgtgcgccaggtgcacgccgacatcaacgccgcccagaacctgcagcgccgcctgtggcagaacttcgacatcaccgagctgcgcctgcgctgcgacgtgaagatgggcggcgagggcaccgtgctggtgccccgcgtgaacaacgcccgcgccaagcagctgttcggcaagaaggtgctggtgagccaggacggcgtgaccttcttcgagcgcagccagaccggcggcaagccccacagcgagaagcagaccgacctgaccgacaaggagctggagctgatcgccgaggccgacgaggcccgcgccaagagcgtggtgctgttccgcgaccccagcggccacatcggcaagggccactggatccgccagcgcgagttctggagcctggtgaagcagcgcatcgagagccacaccgccgagcgcatccgcgtgcgcggcgtgggcagcagcctggac

SEQ ID NO: 13 - DNA, 3438 bp, Unknown
  Alicyclobacillus macrosporangiidus strain DSM 17980
atgaacgtggccgtgaagagcatcaaggtgaagctgatgctgggccacctgcccgagatccgcgagggcctgtggcacctgcacgaggccgtgaacctgggcgtgcgctactacaccgagtggctggccctgctgcgccagggcaacctgtaccgccgcggcaaggacggcgcccaggagtgctacatgaccgccgagcagtgccgccaggagctgctggtgcgcctgcgcgaccgccagaagcgcaacggccacaccggcgaccccggcaccgacgaggagctgctgggcgtggcccgccgcctgtacgagctgctggtgccccagagcgtgggcaagaagggccaggcccagatgctggccagcggcttcctgagccccctggccgaccccaagagcgagggcggcaagggcaccagcaagagcggccgcaagcccgcctggatgggcatgaaggaggccggcgacagccgctgggtggaggccaaggcccgctacgaggccaacaaggccaaggaccccaccaagcaggtgatcgccagcctggagatgtacggcctgcgccccctgttcgacgtgttcaccgagacctacaagaccatccgctggatgcccctgggcaagcaccagggcgtgcgcgcctgggaccgcgacatgttccagcagagcctggagcgcctgatgagctgggagagctggaacgagcgcgtgggcgccgagttcgcccgcctggtggaccgccgcgaccgcttccgcgagaagcacttcaccggccaggagcacctggtggccctggcccagcgcctggagcaggagatgaaggaggccagccccggcttcgagagcaagagcagccaggcccaccgcatcaccaagcgcgccctgcgcggcgccgacggcatcatcgacgactggctgaagctgagcgagggcgagcccgtggaccgcttcgacgagatcctgcgcaagcgccaggcccagaacccccgccgcttcggcagccacgacctgttcctgaagctggccgagcccgtgttccagcccctgtggcgcgaggaccccagcttcctgagccgctgggccagctacaacgaggtgctgaacaagctggaggacgccaagcagttcgccaccttcaccctgcccagcccctgcagcaaccccgtgtgggcccgcttcgagaacgccgagggcaccaacatcttcaagtacgacttcctgttcgaccacttcggcaagggccgccacggcgtgcgcttccagcgcatgatcgtgatgcgcgacggcgtgcccaccgaggtggagggcatcgtggtgcccatcgcccccagccgccagctggacgccctggcccccaacgacgccgccagccccatcgacgtgttcgtgggcgaccccgccgcccccggcgccttccgcggccagttcggcggcgccaagatccagtaccgccgcagcgccctggtgcgcaagggccgccgcgaggagaaggcctacctgtgcggcttccgcctgcccagccagcgccgcaccggcacccccgccgacgacgccggcgaggtgttcctgaacctgagcctgcgcgtggagagccagagcgagcaggccggccgccgcaaccccccctacgccgccgtgttccacatcagcgaccagacccgccgcgtgatcgtgcgctacggcgagatcgagcgctacctggccgagcaccccgacaccggcatccccggcagccgcggcctgaccagcggcctgcgcgtgatgagcgtggacctgggcctgcgcaccagcgccgccatcagcgtgttccgcgtggcccaccgcgacgagctgacccccgacgcccacggccgccagcccttcttcttccccatccacggcatggaccacctggtggccctgcacgagcgcagccacctgatccgcctgcccggcgagaccgagagcaagaaggtgcgcagcatccgcgagcagcgcctggaccgcctgaaccgcctgcgcagccagatggccagcctgcgcctgctggtgcgcaccggcgtgctggacgagcagaagcgcgaccgcaactgggagcgcctgcagagcagcatggagcgcggcggcgagcgcatgcccagcgactggtgggacctgttccaggcccaggtgcgctacctggcccagcaccgcgacgccagcggcgaggcctggggccgcatggtgcaggccgccgtgcgcaccctgtggcgccagctggccaagcaggtgcgcgactggcgcaaggaggtgcgccgcaacgccgacaaggtgaagatccgcggcatcgcccgcgacgtgcccggcggccacagcctggcccagctggactacctggagcgccagtaccgcttcctgcgcagctggagcgccttcagcgtgcaggccggccaggtggtgcgcgccgagcgcgacagccgcttcgccgtggccctgcgcgagcacatcgacaacggcaagaaggaccgcctgaagaagctggccgaccgcatcctgatggaggccctgggctacgtgtacgtgaccgacggccgccgcgccggccagtggcaggccgtgtaccccccctgccagctggtgctgctggaggagctgagcgagtaccgcttcagcaacgaccgcccccccagcgagaacagccagctgatggtgtggagccaccgcggcgtgctggaggagctgatccaccaggcccaggtgcacgacgtgctggtgggcaccatccccgccgccttcagcagccgcttcgacgcccgcaccggcgcccccggcatccgctgccgccgcgtgcccagcatccccctgaaggacgcccccagcatccccatctggctgagccactacctgaagcagaccgagcgcgacgccgccgccctgcgccccggcgagctgatccccaccggcgacggcgagttcctggtgacccccgccggccgcggcgccagcggcgtgcgcgtggtgcacgccgacatcaacgccgcccacaacctgcagcgccgcctgtgggagaacttcgacctgagcgacatccgcgtgcgctgcgaccgccgcgagggcaaggacggcaccgtggtgctgatcccccgcctgaccaaccagcgcgtgaaggagcgctacagcggcgtgatcttcaccagcgaggacggcgtgagcttcaccgtgggcgacgccaagacccgccgccgcagcagcgccagccagggcgagggcgacgacctgagcgacgaggagcaggagctgctggccgaggccgacgacgcccgcgagcgcagcgtggtgctgttccgcgaccccagcggcttcgtgaacggcggccgctggaccgcccagcgcgccttctggggcatggtgcacaaccgcatcgagaccctgctggccgagcgcttcagcgtgagcggcgccgccgagaaggtgcgcggc

SEQ ID NO: 14 - DNA, 3324 bp, Unknown
  Bacillus hisashii strain C4
atggccacccgcagcttcatcctgaagatcgagcccaacgaggaggtgaagaagggcctgtggaagacccacgaggtgctgaaccacggcatcgcctactacatgaacatcctgaagctgatccgccaggaggccatctacgagcaccacgagcaggaccccaagaaccccaagaaggtgagcaaggccgagatccaggccgagctgtgggacttcgtgctgaagatgcagaagtgcaacagcttcacccacgaggtggacaaggacgaggtgttcaacatcctgcgcgagctgtacgaggagctggtgcccagcagcgtggagaagaagggcgaggccaaccagctgagcaacaagttcctgtaccccctggtggaccccaacagccagagcggcaagggcaccgccagcagcggccgcaagccccgctggtacaacctgaagatcgccggcgaccccagctgggaggaggagaagaagaagtgggaggaggacaagaagaaggaccccctggccaagatcctgggcaagctggccgagtacggcctgatccccctgttcatcccctacaccgacagcaacgagcccatcgtgaaggagatcaagtggatggagaagagccgcaaccagagcgtgcgccgcctggacaaggacatgttcatccaggccctggagcgcttcctgagctgggagagctggaacctgaaggtgaaggaggagtacgagaaggtggagaaggagtacaagaccctggaggagcgcatcaaggaggacatccaggccctgaaggccctggagcagtacgagaaggagcgccaggagcagctgctgcgcgacaccctgaacaccaacgagtaccgcctgagcaagcgcggcctgcgcggctggcgcgagatcatccagaagtggctgaagatggacgagaacgagcccagcgagaagtacctggaggtgttcaaggactaccagcgcaagcacccccgcgaggccggcgactacagcgtgtacgagttcctgagcaagaaggagaaccacttcatctggcgcaaccaccccgagtacccctacctgtacgccaccttctgcgagatcgacaagaagaagaaggacgccaagcagcaggccaccttcaccctggccgaccccatcaaccaccccctgtgggtgcgcttcgaggagcgcagcggcagcaacctgaacaagtaccgcatcctgaccgagcagctgcacaccgagaagctgaagaagaagctgaccgtgcagctggaccgcctgatctaccccaccgagagcggcggctgggaggagaagggcaaggtggacatcgtgctgctgcccagccgccagttctacaaccagatcttcctggacatcgaggagaagggcaagcacgccttcacctacaaggacgagagcatcaagttccccctgaagggcaccctgggcggcgcccgcgtgcagttcgaccgcgaccacctgcgccgctacccccacaaggtggagagcggcaacgtgggccgcatctacttcaacatgaccgtgaacatcgagcccaccgagagccccgtgagcaagagcctgaagatccaccgcgacgacttccccaaggtggtgaacttcaagcccaaggagctgaccgagtggatcaaggacagcaagggcaagaagctgaagagcggcatcgagagcctggagatcggcctgcgcgtgatgagcatcgacctgggccagcgccaggccgccgccgccagcatcttcgaggtggtggaccagaagcccgacatcgagggcaagctgttcttccccatcaagggcaccgagctgtacgccgtgcaccgcgccagcttcaacatcaagctgcccggcgagaccctggtgaagagccgcgaggtgctgcgcaaggcccgcgaggacaacctgaagctgatgaaccagaagctgaacttcctgcgcaacgtgctgcacttccagcagttcgaggacatcaccgagcgcgagaagcgcgtgaccaagtggatcagccgccaggagaacagcgacgtgcccctggtgtaccaggacgagctgatccagatccgcgagctgatgtacaagccctacaaggactgggtggccttcctgaagcagctgcacaagcgcctggaggtggagatcggcaaggaggtgaagcactggcgcaagagcctgagcgacggccgcaagggcctgtacggcatcagcctgaagaacatcgacgagatcgaccgcacccgcaagttcctgctgcgctggagcctgcgccccaccgagcccggcgaggtgcgccgcctggagcccggccagcgcttcgccatcgaccagctgaaccacctgaacgccctgaaggaggaccgcctgaagaagatggccaacaccatcatcatgcacgccctgggctactgctacgacgtgcgcaagaagaagtggcaggccaagaaccccgcctgccagatcatcctgttcgaggacctgagcaactacaacccctacgaggagcgcagccgcttcgagaacagcaagctgatgaagtggagccgccgcgagatcccccgccaggtggccctgcagggcgagatctacggcctgcaggtgggcgaggtgggcgcccagttcagcagccgcttccacgccaagaccggcagccccggcatccgctgcagcgtggtgaccaaggagaagctgcaggacaaccgcttcttcaagaacctgcagcgcgagggccgcctgaccctggacaagatcgccgtgctgaaggagggcgacctgtaccccgacaagggcggcgagaagttcatcagcctgagcaaggaccgcaagtgcgtgaccacccacgccgacatcaacgccgcccagaacctgcagaagcgcttctggacccgcacccacggcttctacaaggtgtactgcaaggcctaccaggtggacggccagaccgtgtacatccccgagagcaaggaccagaagcagaagatcatcgaggagttcggcgagggctacttcatcctgaaggacggcgtgtacgagtgggtgaacgccggcaagctgaagatcaagaagggcagcagcaagcagagcagcagcgagctggtggacagcgacatcctgaaggacagcttcgacctggccagcgagctgaagggcgagaagctgatgctgtaccgcgaccccagcggcaacgtgttccccagcgacaagtggatggccgccggcgtgttcttcggcaagctggagcgcatcctgatcagcaagctgaccaaccagtacagcatcagcaccatcgaggacgacagcagcaagcagagcatg

SEQ ID NO: 15 - DNA, 3324 bp, Unknown
  Bacillus sp. NSP2.1
atggccatccgcagcatcaagctgaagctgaagacccacaccggccccgaggcccagaacctgcgcaagggcatctggcgcacccaccgcctgctgaacgagggcgtggcctactacatgaagatgctgctgctgttccgccaggagagcaccggcgagcgccccaaggaggagctgcaggaggagctgatctgccacatccgcgagcagcagcagcgcaaccaggccgacaagaacacccaggccctgcccctggacaaggccctggaggccctgcgccagctgtacgagctgctggtgcccagcagcgtgggccagagcggcgacgcccagatcatcagccgcaagttcctgagccccctggtggaccccaacagcgagggcggcaagggcaccagcaaggccggcgccaagcccacctggcagaagaagaaggaggccaacgaccccacctgggagcaggactacgagaagtggaagaagcgccgcgaggaggaccccaccgccagcgtgatcaccaccctggaggagtacggcatccgccccatcttccccctgtacaccaacaccgtgaccgacatcgcctggctgcccctgcagagcaaccagttcgtgcgcacctgggaccgcgacatgctgcagcaggccatcgagcgcctgctgagctgggagagctggaacaagcgcgtgcaggaggagtacgccaagctgaaggagaagatggcccagctgaacgagcagctggagggcggccaggagtggatcagcctgctggagcagtacgaggagaaccgcgagcgcgagctgcgcgagaacatgaccgccgccaacgacaagtaccgcatcaccaagcgccagatgaagggctggaacgagctgtacgagctgtggagcaccttccccgccagcgccagccacgagcagtacaaggaggccctgaagcgcgtgcagcagcgcctgcgcggccgcttcggcgacgcccacttcttccagtacctgatggaggagaagaaccgcctgatctggaagggcaacccccagcgcatccactacttcgtggcccgcaacgagctgaccaagcgcctggaggaggccaagcagagcgccaccatgaccctgcccaacgcccgcaagcaccccctgtgggtgcgcttcgacgcccgcggcggcaacctgcaggactactacctgaccgccgaggccgacaagccccgcagccgccgcttcgtgaccttcagccagctgatctggcccagcgagagcggctggatggagaagaaggacgtggaggtggagctggccctgagccgccagttctaccagcaggtgaagctgctgaagaacgacaagggcaagcagaagatcgagttcaaggacaagggcagcggcagcaccttcaacggccacctgggcggcgccaagctgcagctggagcgcggcgacctggagaaggaggagaagaacttcgaggacggcgagatcggcagcgtgtacctgaacgtggtgatcgacttcgagcccctgcaggaggtgaagaacggccgcgtgcaggccccctacggccaggtgctgcagctgatccgccgccccaacgagttccccaaggtgaccacctacaagagcgagcagctggtggagtggatcaaggccagcccccagcacagcgccggcgtggagagcctggccagcggcttccgcgtgatgagcatcgacctgggcctgcgcgccgccgccgccaccagcatcttcagcgtggaggagagcagcgacaagaacgccgccgacttcagctactggatcgagggcacccccctggtggccgtgcaccagcgcagctacatgctgcgcctgcccggcgagcaggtggagaagcaggtgatggagaagcgcgacgagcgcttccagctgcaccagcgcgtgaagttccagatccgcgtgctggcccagatcatgcgcatggccaacaagcagtacggcgaccgctgggacgagctggacagcctgaagcaggccgtggagcagaagaagagccccctggaccagaccgaccgcaccttctgggagggcatcgtgtgcgacctgaccaaggtgctgccccgcaacgaggccgactgggagcaggccgtggtgcagatccaccgcaaggccgaggagtacgtgggcaaggccgtgcaggcctggcgcaagcgcttcgccgccgacgagcgcaagggcatcgccggcctgagcatgtggaacatcgaggagctggagggcctgcgcaagctgctgatcagctggagccgccgcacccgcaacccccaggaggtgaaccgcttcgagcgcggccacaccagccaccagcgcctgctgacccacatccagaacgtgaaggaggaccgcctgaagcagctgagccacgccatcgtgatgaccgccctgggctacgtgtacgacgagcgcaagcaggagtggtgcgccgagtaccccgcctgccaggtgatcctgttcgagaacctgagccagtaccgcagcaacctggaccgcagcaccaaggagaacagcaccctgatgaagtgggcccaccgcagcatccccaagtacgtgcacatgcaggccgagccctacggcatccagatcggcgacgtgcgcgccgagtacagcagccgcttctacgccaagaccggcacccccggcatccgctgcaagaaggtgcgcggccaggacctgcagggccgccgcttcgagaacctgcagaagcgcctggtgaacgagcagttcctgaccgaggagcaggtgaagcagctgcgccccggcgacatcgtgcccgacgacagcggcgagctgttcatgaccctgaccgacggcagcggcagcaaggaggtggtgttcctgcaggccgacatcaacgccgcccacaacctgcagaagcgcttctggcagcgctacaacgagctgttcaaggtgagctgccgcgtgatcgtgcgcgacgaggaggagtacctggtgcccaagaccaagagcgtgcaggccaagctgggcaagggcctgttcgtgaagaagagcgacaccgcctggaaggacgtgtacgtgtgggacagccaggccaagctgaagggcaagaccaccttcaccgaggagagcgagagccccgagcagctggaggacttccaggagatcatcgaggaggccgaggaggccaagggcacctaccgcaccctgttccgcgaccccagcggcgtgttcttccccgagagcgtgtggtacccccagaaggacttctggggcgaggtgaagcgcaagctgtacggcaagctgcgcgagcgcttcctgaccaaggcccgc

SEQ ID NO: 16 - DNA, 3336 bp, Unknown
  Bacillus sp. V3-13
atggccatccgcagcatcaagctgaagatgaagaccaacagcggcaccgacagcatctacctgcgcaaggccctgtggcgcacccaccagctgatcaacgagggcatcgcctactacatgaacctgctgaccctgtaccgccaggaggccatcggcgacaagaccaaggaggcctaccaggccgagctgatcaacatcatccgcaaccagcagcgcaacaacggcagcagcgaggagcacggcagcgaccaggagatcctggccctgctgcgccagctgtacgagctgatcatccccagcagcatcggcgagagcggcgacgccaaccagctgggcaacaagttcctgtaccccctggtggaccccaacagccagagcggcaagggcaccagcaacgccggccgcaagccccgctggaagcgcctgaaggaggagggcaaccccgactgggagctggagaagaagaaggacgaggagcgcaaggccaaggaccccaccgtgaagatcttcgacaacctgaacaagtacggcctgctgcccctgttccccctgttcaccaacatccagaaggacatcgagtggctgcccctgggcaagcgccagagcgtgcgcaagtgggacaaggacatgttcatccaggccatcgagcgcctgctgagctgggagagctggaaccgccgcgtggccgacgagtacaagcagctgaaggagaagaccgagagctactacaaggagcacctgaccggcggcgaggagtggatcgagaagatccgcaagttcgagaaggagcgcaacatggagctggagaagaacgccttcgcccccaacgacggctacttcatcaccagccgccagatccgcggctgggaccgcgtgtacgagaagtggagcaagctgcccgagagcgccagccccgaggagctgtggaaggtggtggccgagcagcagaacaagatgagcgagggcttcggcgaccccaaggtgttcagcttcctggccaaccgcgagaaccgcgacatctggcgcggccacagcgagcgcatctaccacatcgccgcctacaacggcctgcagaagaagctgagccgcaccaaggagcaggccaccttcaccctgcccgacgccatcgagcaccccctgtggatccgctacgagagccccggcggcaccaacctgaacctgttcaagctggaggagaagcagaagaagaactactacgtgaccctgagcaagatcatctggcccagcgaggagaagtggatcgagaaggagaacatcgagatccccctggcccccagcatccagttcaaccgccagatcaagctgaagcagcacgtgaagggcaagcaggagatcagcttcagcgactacagcagccgcatcagcctggacggcgtgctgggcggcagccgcatccagttcaaccgcaagtacatcaagaaccacaaggagctgctgggcgagggcgacatcggccccgtgttcttcaacctggtggtggacgtggcccccctgcaggagacccgcaacggccgcctgcagagccccatcggcaaggccctgaaggtgatcagcagcgacttcagcaaggtgatcgactacaagcccaaggagctgatggactggatgaacaccggcagcgccagcaacagcttcggcgtggccagcctgctggagggcatgcgcgtgatgagcatcgacatgggccagcgcaccagcgccagcgtgagcatcttcgaggtggtgaaggagctgcccaaggaccaggagcagaagctgttctacagcatcaacgacaccgagctgttcgccatccacaagcgcagcttcctgctgaacctgcccggcgaggtggtgaccaagaacaacaagcagcagcgccaggagcgccgcaagaagcgccagttcgtgcgcagccagatccgcatgctggccaacgtgctgcgcctggagaccaagaagacccccgacgagcgcaagaaggccatccacaagctgatggagatcgtgcagagctacgacagctggaccgccagccagaaggaggtgtgggagaaggagctgaacctgctgaccaacatggccgccttcaacgacgagatctggaaggagagcctggtggagctgcaccaccgcatcgagccctacgtgggccagatcgtgagcaagtggcgcaagggcctgagcgagggccgcaagaacctggccggcatcagcatgtggaacatcgacgagctggaggacacccgccgcctgctgatcagctggagcaagcgcagccgcacccccggcgaggccaaccgcatcgagaccgacgagcccttcggcagcagcctgctgcagcacatccagaacgtgaaggacgaccgcctgaagcagatggccaacctgatcatcatgaccgccctgggcttcaagtacgacaaggaggagaaggaccgctacaagcgctggaaggagacctaccccgcctgccagatcatcctgttcgagaacctgaaccgctacctgttcaacctggaccgcagccgccgcgagaacagccgcctgatgaagtgggcccaccgcagcatcccccgcaccgtgagcatgcagggcgagatgttcggcctgcaggtgggcgacgtgcgcagcgagtacagcagccgcttccacgccaagaccggcgcccccggcatccgctgccacgccctgaccgaggaggacctgaaggccggcagcaacaccctgaagcgcctgatcgaggacggcttcatcaacgagagcgagctggcctacctgaagaagggcgacatcatccccagccagggcggcgagctgttcgtgaccctgagcaagcgctacaagaaggacagcgacaacaacgagctgaccgtgatccacgccgacatcaacgccgcccagaacctgcagaagcgcttctggcagcagaacagcgaggtgtaccgcgtgccctgccagctggcccgcatgggcgaggacaagctgtacatccccaagagccagaccgagaccatcaagaagtacttcggcaagggcagcttcgtgaagaacaacaccgagcaggaggtgtacaagtgggagaagagcgagaagatgaagatcaagaccgacaccaccttcgacctgcaggacctggacggcttcgaggacatcagcaagaccatcgagctggcccaggagcagcagaagaagtacctgaccatgttccgcgaccccagcggctacttcttcaacaacgagacctggcgcccccagaaggagtactggagcatcgtgaacaacatcatcaagagctgcctgaagaagaagatcctgagcaacaaggtggagctg

SEQ ID NO: 17 - DNA, 3447 bp, Unknown
  Desulfovibrio inopinatus DSM 10711
atgcccacccgcaccatcaacctgaagctggtgctgggcaagaaccccgagaacgccaccctgcgccgcgccctgttcagcacccaccgcctggtgaaccaggccaccaagcgcatcgaggagttcctgctgctgtgccgcggcgaggcctaccgcaccgtggacaacgagggcaaggaggccgagatcccccgccacgccgtgcaggaggaggccctggccttcgccaaggccgcccagcgccacaacggctgcatcagcacctacgaggaccaggagatcctggacgtgctgcgccagctgtacgagcgcctggtgcccagcgtgaacgagaacaacgaggccggcgacgcccaggccgccaacgcctgggtgagccccctgatgagcgccgagagcgagggcggcctgagcgtgtacgacaaggtgctggaccccccccccgtgtggatgaagctgaaggaggagaaggcccccggctgggaggccgccagccagatctggatccagagcgacgagggccagagcctgctgaacaagcccggcagccccccccgctggatccgcaagctgcgcagcggccagccctggcaggacgacttcgtgagcgaccagaagaagaagcaggacgagctgaccaagggcaacgcccccctgatcaagcagctgaaggagatgggcctgctgcccctggtgaaccccttcttccgccacctgctggaccccgagggcaagggcgtgagcccctgggaccgcctggccgtgcgcgccgccgtggcccacttcatcagctgggagagctggaaccaccgcacccgcgccgagtacaacagcctgaagctgcgccgcgacgagttcgaggccgccagcgacgagttcaaggacgacttcaccctgctgcgccagtacgaggccaagcgccacagcaccctgaagagcatcgccctggccgacgacagcaacccctaccgcatcggcgtgcgcagcctgcgcgcctggaaccgcgtgcgcgaggagtggatcgacaagggcgccaccgaggagcagcgcgtgaccatcctgagcaagctgcagacccagctgcgcggcaagttcggcgaccccgacctgttcaactggctggcccaggaccgccacgtgcacctgtggagcccccgcgacagcgtgacccccctggtgcgcatcaacgccgtggacaaggtgctgcgccgccgcaagccctacgccctgatgaccttcgcccacccccgcttccacccccgctggatcctgtacgaggcccccggcggcagcaacctgcgccagtacgccctggactgcaccgagaacgccctgcacatcaccctgcccctgctggtggacgacgcccacggcacctggatcgagaagaagatccgcgtgcccctggcccccagcggccagatccaggacctgaccctggagaagctggagaagaagaagaaccgcctgtactaccgcagcggcttccagcagttcgccggcctggccggcggcgccgaggtgctgttccaccgcccctacatggagcacgacgagcgcagcgaggagagcctgctggagcgccccggcgccgtgtggttcaagctgaccctggacgtggccacccaggccccccccaactggctggacggcaagggccgcgtgcgcaccccccccgaggtgcaccacttcaagaccgccctgagcaacaagagcaagcacacccgcaccctgcagcccggcctgcgcgtgctgagcgtggacctgggcatgcgcaccttcgccagctgcagcgtgttcgagctgatcgagggcaagcccgagaccggccgcgccttccccgtggccgacgagcgcagcatggacagccccaacaagctgtgggccaagcacgagcgcagcttcaagctgaccctgcccggcgagacccccagccgcaaggaggaggaggagcgcagcatcgcccgcgccgagatctacgccctgaagcgcgacatccagcgcctgaagagcctgctgcgcctgggcgaggaggacaacgacaaccgccgcgacgccctgctggagcagttcttcaagggctggggcgaggaggacgtggtgcccggccaggccttcccccgcagcctgttccagggcctgggcgccgcccccttccgcagcacccccgagctgtggcgccagcactgccagacctactacgacaaggccgaggcctgcctggccaagcacatcagcgactggcgcaagcgcacccgcccccgccccaccagccgcgagatgtggtacaagacccgcagctaccacggcggcaagagcatctggatgctggagtacctggacgccgtgcgcaagctgctgctgagctggagcctgcgcggccgcacctacggcgccatcaaccgccaggacaccgcccgcttcggcagcctggccagccgcctgctgcaccacatcaacagcctgaaggaggaccgcatcaagaccggcgccgacagcatcgtgcaggccgcccgcggctacatccccctgccccacggcaagggctgggagcagcgctacgagccctgccagctgatcctgttcgaggacctggcccgctaccgcttccgcgtggaccgcccccgccgcgagaacagccagctgatgcagtggaaccaccgcgccatcgtggccgagaccaccatgcaggccgagctgtacggccagatcgtggagaacaccgccgccggcttcagcagccgcttccacgccgccaccggcgcccccggcgtgcgctgccgcttcctgctggagcgcgacttcgacaacgacctgcccaagccctacctgctgcgcgagctgagctggatgctgggcaacaccaaggtggagagcgaggaggagaagctgcgcctgctgagcgagaagatccgccccggcagcctggtgccctgggacggcggcgagcagttcgccaccctgcaccccaagcgccagaccctgtgcgtgatccacgccgacatgaacgccgcccagaacctgcagcgccgcttcttcggccgctgcggcgaggccttccgcctggtgtgccagccccacggcgacgacgtgctgcgcctggccagcacccccggcgcccgcctgctgggcgccctgcagcagctggagaacggccagggcgccttcgagctggtgcgcgacatgggcagcaccagccagatgaaccgcttcgtgatgaagagcctgggcaagaagaagatcaagcccctgcaggacaacaacggcgacgacgagctggaggacgtgctgagcgtgctgcccgaggaggacgacaccggccgcatcaccgtgttccgcgacagcagcggcatcttcttcccctgcaacgtgtggatccccgccaagcagttctggcccgccgtgcgcgccatgatctggaaggtgatggccagccacagcctgggc

SEQ ID NO: 18 - DNA, 3270 bp, Unknown
  Laceyella sediminis strain RHA1
atgagcatccgcagcttcaagctgaagatcaagaccaagagcggcgtgaacgccgaggagctgcgccgcggcctgtggcgcacccaccagctgatcaacgacggcatcgcctactacatgaactggctggtgctgctgcgccaggaggacctgttcatccgcaacgaggagaccaacgagatcgagaagcgcagcaaggaggagatccagggcgagctgctggagcgcgtgcacaagcagcagcagcgcaaccagtggagcggcgaggtggacgaccagaccctgctgcagaccctgcgccacctgtacgaggagatcgtgcccagcgtgatcggcaagagcggcaacgccagcctgaaggcccgcttcttcctgggccccctggtggaccccaacaacaagaccaccaaggacgtgagcaagagcggccccacccccaagtggaagaagatgaaggacgccggcgaccccaactgggtgcaggagtacgagaagtacatggccgagcgccagaccctggtgcgcctggaggagatgggcctgatccccctgttccccatgtacaccgacgaggtgggcgacatccactggctgccccaggccagcggctacacccgcacctgggaccgcgacatgttccagcaggccatcgagcgcctgctgagctgggagagctggaaccgccgcgtgcgcgagcgccgcgcccagttcgagaagaagacccacgacttcgccagccgcttcagcgagagcgacgtgcagtggatgaacaagctgcgcgagtacgaggcccagcaggagaagagcctggaggagaacgccttcgcccccaacgagccctacgccctgaccaagaaggccctgcgcggctgggagcgcgtgtaccacagctggatgcgcctggacagcgccgccagcgaggaggcctactggcaggaggtggccacctgccagaccgccatgcgcggcgagttcggcgaccccgccatctaccagttcctggcccagaaggagaaccacgacatctggcgcggctaccccgagcgcgtgatcgacttcgccgagctgaaccacctgcagcgcgagctgcgccgcgccaaggaggacgccaccttcaccctgcccgacagcgtggaccaccccctgtgggtgcgctacgaggcccccggcggcaccaacatccacggctacgacctggtgcaggacaccaagcgcaacctgaccctgatcctggacaagttcatcctgcccgacgagaacggcagctggcacgaggtgaagaaggtgcccttcagcctggccaagagcaagcagttccaccgccaggtgtggctgcaggaggagcagaagcagaagaagcgcgaggtggtgttctacgactacagcaccaacctgccccacctgggcaccctggccggcgccaagctgcagtgggaccgcaacttcctgaacaagcgcacccagcagcagatcgaggagaccggcgagatcggcaaggtgttcttcaacatcagcgtggacgtgcgccccgccgtggaggtgaagaacggccgcctgcagaacggcctgggcaaggccctgaccgtgctgacccaccccgacggcaccaagatcgtgaccggctggaaggccgagcagctggagaagtgggtgggcgagagcggccgcgtgagcagcctgggcctggacagcctgagcgagggcctgcgcgtgatgagcatcgacctgggccagcgcaccagcgccaccgtgagcgtgttcgagatcaccaaggaggcccccgacaacccctacaagttcttctaccagctggagggcaccgagctgttcgccgtgcaccagcgcagcttcctgctggccctgcccggcgagaaccccccccagaagatcaagcagatgcgcgagatccgctggaaggagcgcaaccgcatcaagcagcaggtggaccagctgagcgccatcctgcgcctgcacaagaaggtgaacgaggacgagcgcatccaggccatcgacaagctgctgcagaaggtggccagctggcagctgaacgaggagatcgccaccgcctggaaccaggccctgagccagctgtacagcaaggccaaggagaacgacctgcagtggaaccaggccatcaagaacgcccaccaccagctggagcccgtggtgggcaagcagatcagcctgtggcgcaaggacctgagcaccggccgccagggcatcgccggcctgagcctgtggagcatcgaggagctggaggccaccaagaagctgctgacccgctggagcaagcgcagccgcgagcccggcgtggtgaagcgcatcgagcgcttcgagaccttcgccaagcagatccagcaccacatcaaccaggtgaaggagaaccgcctgaagcagctggccaacctgatcgtgatgaccgccctgggctacaagtacgaccaggagcagaagaagtggatcgaggtgtaccccgcctgccaggtggtgctgttcgagaacctgcgcagctaccgcttcagctacgagcgcagccgccgcgagaacaagaagctgatggagtggagccaccgcagcatccccaagctggtgcagatgcagggcgagctgttcggcctgcaggtggccgacgtgtacgccgcctacagcagccgctaccacggccgcaccggcgcccccggcatccgctgccacgccctgaccgaggccgacctgcgcaacgagaccaacatcatccacgagctgatcgaggccggcttcatcaaggaggagcaccgcccctacctgcagcagggcgacctggtgccctggagcggcggcgagctgttcgccaccctgcagaagccctacgacaacccccgcatcctgaccctgcacgccgacatcaacgccgcccagaacatccagaagcgcttctggcaccccagcatgtggttccgcgtgaactgcgagagcgtgatggagggcgagatcgtgacctacgtgcccaagaacaagaccgtgcacaagaagcagggcaagaccttccgcttcgtgaaggtggagggcagcgacgtgtacgagtgggccaagtggagcaagaaccgcaacaagaacaccttcagcagcatcaccgagcgcaagccccccagcagcatgatcctgttccgcgaccccagcggcaccttcttcaaggagcaggagtgggtggagcagaagaccttctggggcaaggtgcagagcatgatccaggcctacatgaagaagaccatcgtgcagcgcatggaggag

SEQ ID NO: 19 - DNA, 3357 bp, Unknown
  Spirochaetes bacterium GWB1_27_13
atgagcttcaccatcagctaccccttcaagctgatcatcaagaacaaggacgaggccaaggccctgctggacacccaccagtacatgaacgagggcgtgaagtactacctggagaagctgctgatgttccgccaggagaagatcttcatcggcgaggacgagaccggcaagcgcatctacatcgaggagaccgagtacaagaagcagatcgaggagttctacctgatcaagaagaccgagctgggccgcaacctgaccctgaccctggacgagttcaagaccctgatgcgcgagctgtacatctgcctggtgagcagcagcatggagaacaagaagggcttccccaacgcccagcaggccagcctgaacatcttcagccccctgttcgacgccgagagcaagggctacatcctgaaggaggagaacaacaacatcagcctgatccacaaggactacggcaagatcctgctgaagcgcctgcgcgacaacaacctgatccccatcttcaccaagttcaccgacatcaagaagatcaccgccaagctgagccccaccgccctggaccgcatgatcttcgcccaggccatcgagaagctgctgagctacgagagctggtgcaagctgatgatcaaggagcgcttcgacaaggaggtgaagatcaaggagctggagaacaagtgcgagaacaagcaggagcgcgacaagatcttcgagatcctggagaagtacgaggaggagcgccagaagaccttcgagcaggacagcggcttcgccaagaagggcaagttctacatcaccggccgcatgctgaagggcttcgacgagatcaaggagaagtggctgaaggagaaggaccgcagcgagcagaacctgatcaacatcctgaacaagtaccagaccgacaacagcaagctggtgggcgaccgcaacctgttcgagttcatcatcaagctggagaaccagtgcctgtggaacggcgacatcgactacctgaagatcaagcgcgacatcaacaagaaccagatctggctggaccgccccgagatgccccgcttcaccatgcccgacttcaagaagcaccccctgtggtaccgctacgaggaccccagcaacagcaacttccgcaactacaagatcgaggtggtgaaggacgagaactacatcaccatccccctgatcaccgagcgcaacaacgagtacttcgaggagaactacaccttcaacctggccaagctgaagaagctgagcgagaacatcaccttcatccccaagagcaagaacaaggagttcgagttcatcgacagcaacgacgaggaggaggacaagaaggaccagaagaagagcaagcagtacatcaagtactgcgacaccgccaagaacaccagctacggcaagagcggcggcatccgcctgtacttcaaccgcaacgagctggagaactacaaggacggcaagaagatggacagctacaccgtgttcaccctgagcatccgcgactacaagagcctgttcgccaaggagaagctgcagccccagatcttcaacaccgtggacaacaagatcaccagcctgaagatccagaagaagttcggcaacgaggagcagaccaacttcctgagctacttcacccagaaccagatcaccaagaaggactggatggacgagaagaccttccagaacgtgaaggagctgaacgagggcatccgcgtgctgagcgtggacctgggccagcgcttcttcgccgccgtgagctgcttcgagatcatgagcgagatcgacaacaacaagctgttcttcaacctgaacgaccagaaccacaagatcatccgcatcaacgacaagaactactacgccaagcacatctacagcaagaccatcaagctgagcggcgaggacgacgacctgtacaaggagcgcaagatcaacaagaactacaagctgagctaccaggagcgcaagaacaagatcggcatcttcacccgccagatcaacaagctgaaccagctgctgaagatcatccgcaacgacgagatcgacaaggagaagttcaaggagctgatcgagaccaccaagcgctacgtgaagaacacctacaacgacggcatcatcgactggaacaacgtggacaacaagatcctgagctacgagaacaaggaggacgtgatcaacctgcacaaggagctggacaagaagctggagatcgacttcaaggagttcatccgcgagtgccgcaagcccatcttccgcagcggcggcctgagcatgcagcgcatcgacttcctggagaagctgaacaagctgaagcgcaagtgggtggcccgcacccagaagagcgccgagagcatcgtgctgacccccaagttcggctacaagctgaaggagcacatcaacgagctgaaggacaaccgcgtgaagcagggcgtgaactacatcctgatgaccgccctgggctacatcaaggacaacgagatcaagaacgacagcaagaagaagcagaaggaggactgggtgaagaagaaccgcgcctgccagatcatcctgatggagaagctgaccgagtacaccttcgccgaggaccgcccccgcgaggagaacagcaagctgcgcatgtggagccaccgccagatcttcaacttcctgcagcagaaggccagcctgtggggcatcctggtgggcgacgtgttcgccccctacaccagcaagtgcctgagcgacaacaacgcccccggcatccgctgccaccaggtgaccaagaaggacctgatcgacaacagctggttcctgaagatcgtggtgaaggacgacgccttctgcgacctgatcgagatcaacaaggagaacgtgaagaacaagagcatcaagatcaacgacatcctgcccctgcgcggcggcgagctgttcgccagcatcaaggacggcaagctgcacatcgtgcaggccgacatcaacgccagccgcaacatcgccaagcgcttcctgagccagatcaaccccttccgcgtggtgctgaagaaggacaaggacgagaccttccacctgaagaacgagcccaactacctgaagaactactacagcatcctgaacttcgtgcccaccaacgaggagctgaccttcttcaaggtggaggagaacaaggacatcaagcccaccaagcgcatcaagatggacaagcacgagaaggagagcaccgacgagggcgacgactacagcaagaaccagatcgccctgttccgcgacgacagcggcatcttcttcgacaagagcctgtgggtggacggcaagatcttctggagcgtggtgaagaacaagatgaccaagctgctgcgcgagcgcaacaacaagaagaacggcagcaag

SEQ ID NO: 20 - DNA, 3426 bp, Unknown
  Tuberibacillus calidus DSM 17572
atgaacatccacctgaaggagctgatccgcatggccaccaagagcttcatcctgaagatgaagaccaagaacaacccccagctgcgcctgagcctgtggaagacccacgagctgttcaacttcggcgtggcctactacatggacctgctgagcctgttccgccagaaggacctgtacatgcacaacgacgaggaccccgaccaccccgtggtgctgaagaaggaggagatccaggagcgcctgtggatgaaggtgcgcgagacccagcagaagaacggcttccacggcgaggtgagcaaggacgaggtgctggagaccctgcgcgccctgtacgaggagctggtgcccagcgccgtgggcaagagcggcgaggccaaccagatcagcaacaagtacctgtaccccctgaccgaccccgccagccagagcggcaagggcaccgccaacagcggccgcaagccccgctggaagaagctgaaggaggccggcgaccccagctggaaggacgcctacgagaagtgggagaaggagcgccaggaggaccccaagctgaagatcctggccgccctgcagagcttcggcctgatccccctgttccgccccttcaccgagaacgaccacaaggccgtgatcagcgtgaagtggatgcccaagagcaagaaccagagcgtgcgcaagttcgacaaggacatgttcaaccaggccatcgagcgcttcctgagctgggagagctggaacgagaaggtggccgaggactacgagaagaccgtgagcatctacgagagcctgcagaaggagctgaagggcatcagcaccaaggccttcgagatcatggagcgcgtggagaaggcctacgaggcccacctgcgcgagatcaccttcagcaacagcacctaccgcatcggcaaccgcgccatccgcggctggaccgagatcgtgaagaagtggatgaagctggaccccagcgccccccagggcaactacctggacgtggtgaaggactaccagcgccgccacccccgcgagagcggcgacttcaagctgttcgagctgctgagccgccccgagaaccaggccgcctggcgcgagtaccccgagttcctgcccctgtacgtgaagtaccgccacgccgagcagcgcatgaagaccgccaagaagcaggccaccttcaccctgtgcgaccccatccgccaccccctgtgggtgcgctacgaggagcgcagcggcaccaacctgaacaagtaccgcctgatcatgaacgagaaggagaaggtggtgcagttcgaccgcctgatctgcctgaacgccgacggccactacgaggagcaggaggacgtgaccgtgcccctggcccccagccagcagttcgacgaccagatcaagttcagcagcgaggacaccggcaagggcaagcacaacttcagctactaccacaagggcatcaactacgagctgaagggcaccctgggcggcgcccgcatccagttcgaccgcgagcacctgctgcgccgccagggcgtgaaggccggcaacgtgggccgcatcttcctgaacgtgaccctgaacatcgagcccatgcagcccttcagccgcagcggcaacctgcagaccagcgtgggcaaggccctgaaggtgtacgtggacggctaccccaaggtggtgaacttcaagcccaaggagctgaccgagcacatcaaggagagcgagaagaacaccctgaccctgggcgtggagagcctgcccaccggcctgcgcgtgatgagcgtggacctgggccagcgccaggccgccgccatcagcatcttcgaggtggtgagcgagaagcccgacgacaacaagctgttctaccccgtgaaggacaccgacctgttcgccgtgcaccgcaccagcttcaacatcaagctgcccggcgagaagcgcaccgagcgccgcatgctggagcagcagaagcgcgaccaggccatccgcgacctgagccgcaagctgaagttcctgaagaacgtgctgaacatgcagaagctggagaagaccgacgagcgcgagaagcgcgtgaaccgctggatcaaggaccgcgagcgcgaggaggagaaccccgtgtacgtgcaggagttcgagatgatcagcaaggtgctgtacagcccccacagcgtgtgggtggaccagctgaagagcatccaccgcaagctggaggagcagctgggcaaggagatcagcaagtggcgccagagcatcagccagggccgccagggcgtgtacggcatcagcctgaagaacatcgaggacatcgagaagacccgccgcctgctgttccgctggagcatgcgccccgagaaccccggcgaggtgaagcagctgcagcccggcgagcgcttcgccatcgaccagcagaaccacctgaaccacctgaaggacgaccgcatcaagaagctggccaaccagatcgtgatgaccgccctgggctaccgctacgacggcaagcgcaagaagtggatcgccaagcaccccgcctgccagctggtgctgttcgaggacctgagccgctacgccttctacgacgagcgcagccgcctggagaaccgcaacctgatgcgctggagccgccgcgagatccccaagcaggtggcccagatcggcggcctgtacggcctgctggtgggcgaggtgggcgcccagtacagcagccgcttccacgccaagagcggcgcccccggcatccgctgccgcgtggtgaaggagcacgagctgtacatcaccgagggcggccagaaggtgcgcaaccagaagttcctggacagcctggtggagaacaacatcatcgagcccgacgacgcccgccgcctggagcccggcgacctgatccgcgaccagggcggcgacaagttcgccaccctggacgagcgcggcgagctggtgatcacccacgccgacatcaacgccgcccagaacctgcagaagcgcttctggacccgcacccacggcctgtaccgcatccgctgcgagagccgcgagatcaaggacgccgtggtgctggtgcccagcgacaaggaccagaaggagaagatggagaacctgttcggcatcggctacctgcagcccttcaagcaggagaacgacgtgtacaagtgggtgaagggcgagaagatcaagggcaagaagaccagcagccagagcgacgacaaggagctggtgagcgagatcctgcaggaggcgagcgtgatggccgacgagctgaagggcaaccgcaagaccctgttccgcgaccccagcggctacgtgttccccaaggaccgctggtacaccggcggccgctacttcggcaccctggagcacctgctgaagcgcaagctggccgagcgccgcctgttcgacggcggcagcagccgccgcggcctgttcaacggcaccgacagcaacaccaacgtggag

SEQ ID NO: 21 - DNA, 2870 bp, Artificial Sequence
  pCAG-2AeGFP (CAG-NLS-XmaI-NheI-NLS-T2A-eGFP-SV40)
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccatctccagcctcggggctgtccgcagggggacggctgccttcgggggggacggggcagggcggggttcggcttctggcgtgtgaccggcggctctagcgcctctgctaaccatgttcatgccttcttctttttcctacagctcctgggcaacgtgctggttattgtgctgtctcatcattttggcaaagctagtgaattctaatacgactcactataggccgccaccatgcccaagaagaagaggaaggttcccggggctagcccaaagaagaagaggaaagtctctagatacccttatgatgttccagattatgccggatacccatacgatgtccctgactatgcaggctcctacccttatgacgtcccagactacgccggatccaggtccggcggcggagagggcagaggaagtcttctaacatgcggtgacgtggaggagaatcccggcccaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaactgcagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatctta

SEQ ID NO: 22 - DNA, 410 bp, Artificial Sequence
  pUC19-U6 (U6-BasI-HindIII)
  misc_feature (283)..(289): n is a, c, g, or t
  misc_feature (297)..(375): n is a, c, g, or t
tgtaaaacgacggccagtgaattcgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggagagaccnnnnnnnggtctcannnnnnnnnnnnnnnnnnnnnnnnnnnnnnnnnnnnnnnnnnnnnnnnnnnnnnnnnnnnnnnnnnnnnnnnnnnnnnnaagcttggcgtaatcatggtcatagctgtttcctg

SEQ ID NO: 23 - DNA, 476 bp, Artificial Sequence
  pUC19-U6-AasgRNA (U6-AasgRNA_ scaffold-BasI-BasI-terminator)
tgtaaaacgacggccagtgaattcgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgggtctaaaggacagaatttttcaacgggtgtgccaatggccactttccaggtggcaaagcccgttgaacttctcaaaaagaacgctcgctcagtgttctgacgtcggatcactgagcgagcgatctgagaagtggcacagagaccgagagagggtctcattttttttaagcttggcgtaatcatggtcatagctgtttcctg

SEQ ID NO: 24 - DNA, 476 bp, Artificial Sequence
  pUC19-U6-AksgRNA(U6-AksgRNA1_ scaffold-BasI-BasI-)
tgtaaaacgacggccagtgaattcgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggtcgtctataggacggcgaggacaacgggaagtgccaatgtgctctttccaagagcaaacaccccgttggcttcaagatgaccgctcgctcagcgatctgacaacggatcgctgagcgagcggtctgagaagtggcacagagaccgagagagggtctcattttttttaagcttggcgtaatcatggtcatagctgtttcctg

SEQ ID NO: 25 - DNA, 484 bp, Artificial Sequence
  pUC19-U6-AmsgRNA(U6-AmsgRNA1_ scaffold-BasI-BasI-)
tgtaaaacgacggccagtgaattcgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggggaattgccgatctataggacggcagattcaacgggatgtgccaatgcactctttccaggagtgaacaccccgttggcttcaacatgatcgcccgctcaacggtccgatgtcggatcgttgagcgggcgatctgagaagtggcacagagaccgagagagggtctcattttttttaagcttggcgtaatcatggtcatagctgtttcctg

SEQ ID NO: 26 - DNA, 439 bp, Artificial Sequence
  pUC19-U6-BhsgRNA (U6-BhsgRNA1_ scaffold-BasI-BasI-)
tgtaaaacgacggccagtgaattcgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgggaggttctgtcttttggtcaggacaaccgtctagctataagtgctgcagggtgtgagaaactcctattgctggacgatgtctcttacgaggcattagcacagagaccgagagagggtctcattttttttaagcttggcgtaatcatggtcatagctgtttcctg

SEQ ID NO: 27 - DNA, 471 bp, Artificial Sequence
  pUC19-U6-BssgRNA (U6-BssgRNA1_ scaffold-BasI-BasI-)
tgtaaaacgacggccagtgaattcgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggccataagtcgacttacatatccgtgcgtgtgcattatgggcccatccacaggtctattcccacggataatcacgactttccactaagctttcgaatgttcgaaagcttagtggaaagcttcgtggttagcacagagaccgagagagggtctcattttttttaagcttggcgtaatcatggtcatagctgtttcctg

SEQ ID NO: 28 - DNA, 469 bp, Artificial Sequence
  pUC19-U6-Bs3sgRNA(U6-BssgRNA1_ scaffold-BasI-BasI-)
tgtaaaacgacggccagtgaattcgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggggtgacctatagggtcaatgaatctgtgcgtgtgccataagtaattaaaaattacccaccacaggattatcttatttctgctaagtgtttagttgcctgaatacttagcagaaataatgatgattggcacagagaccgagagagggtctcattttttttaagcttggcgtaatcatggtcatagctgtttcctg

SEQ ID NO: 29 - DNA, 457 bp, Artificial Sequence
  pUC19-U6-LssgRNA (U6-BssgRNA1_ scaffold-BasI-BasI-)
tgtaaaacgacggccagtgaattcgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggggcaaagaatactgtgcgtgtgctaaggatggaaaaaatccattcaaccacaggattacattatttatctaatcacttaaatctttaagtgattagatgaattaaatgtgattagcacagagaccgagagagggtctcattttttttaagcttggcgtaatcatggtcatagctgtttcctg

SEQ ID NO: 30 - DNA, 465 bp, Artificial Sequence
  pUC19-U6-SbsgRNA (U6-BssgRNA1_ scaffold-BasI-BasI-)
tgtaaaacgacggccagtgaattcgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgggtcttagggtatatcccaaatttgtcttagtatgtgcattgcttacagcgacaactaaggtttgtttatcttttttttacattgtaagatgttttacattataaaaagaagataatcttattgcacagagaccgagagagggtctcattttttttaagcttggcgtaatcatggtcatagctgtttcctg

SEQ ID NO: 31 - DNA, 172 bp, Artificial Sequence
  AasgRNA_ scaffold
  variation (138)..(172): Nucleotides 138-172 can either all be missing or up to 17 n′s can be missing
  misc_feature (138)..(172): n is a, c, g, or t
gtctaaaggacagaatttttcaacgggtgtgccaatggccactttccaggtggcaaagcccgttgaacttctcaaaaagaacgctcgctcagtgttctgacgtcggatcactgagcgagcgatctgagaagtggcacnnnnnnnnnnnnnnnnnnnnnnnnnnnnnnnnnnn

SEQ ID NO: 32 - DNA, 172 bp, Artificial Sequence
  AksgRNA1_ scaffold
  variation (138)..(172): Nucleotides 138-172 can either all be missing or up to 17 n′s can be missing
  misc_feature (138)..(172): n is a, c, g, or t
tcgtctataggacggcgaggacaacgggaagtgccaatgtgctctttccaagagcaaacaccccgttggcttcaagatgaccgctcgctcagcgatctgacaacggatcgctgagcgagcggtctgagaagtggcacnnnnnnnnnnnnnnnnnnnnnnnnnnnnnnnnnnn

SEQ ID NO: 33 - DNA, 180 bp, Artificial Sequence
  AmsgRNA1_ scaffold
  variation (146)..(180): Nucleotides 146-180 can either all be missing or up to 17 n′s can be missing
  misc_feature (146)..(180): n is a, c, g, or t
ggaattgccgatctataggacggcagattcaacgggatgtgccaatgcactctttccaggagtgaacaccccgttggcttcaacatgatcgcccgctcaacggtccgatgtcggatcgttgagcgggcgatctgagaagtggcacnnnnnnnnnnnnnnnnnnnnnnnnnnnnnnnnnnn

SEQ ID NO: 34 - DNA, 176 bp, Artificial Sequence
  BhsgRNA_ scaffold
  variation (142)..(176): Nucleotides 142-176 can either all be missing or up to 17 n′s can be missing
  misc_feature (142)..(176): n is a, c, g, or t
gaggttctgtcttttggtcaggacaaccgtctagctataagtgctgcagggtgtgagaaactcctattgctggacgatgtctcttttatttcttttttcttggatgtccaagaaaaaagaaatgatacgaggcattagcacnnnnnnnnnnnnnnnnnnnnnnnnnnnnnnnnnnn

SEQ ID NO: 35 - DNA, 167 bp, Artificial Sequence
  BssgRNA_ scaffold
  variation (133)..(167): Nucleotides 133-167 can either all be missing or up to 17 n′s can be missing
  misc_feature (133)..(167): n is a, c, g, or t
ccataagtcgacttacatatccgtgcgtgtgcattatgggcccatccacaggtctattcccacggataatcacgactttccactaagctttcgaatgttcgaaagcttagtggaaagcttcgtggttagcacnnnnnnnnnnnnnnnnnnnnnnnnnnnnnnnnnnn

SEQ ID NO: 36 - DNA, 165 bp, Artificial Sequence
  Bs3sgRNA_ scaffold
  variation (131)..(165): Nucleotides 131-165 can either all be missing or up to 17 n′s can be missing
  misc_feature (131)..(165): n is a, c, g, or t
ggtgacctatagggtcaatgaatctgtgcgtgtgccataagtaattaaaaattacccaccacaggattatcttatttctgctaagtgtttagttgcctgaatacttagcagaaataatgatgattggcacnnnnnnnnnnnnnnnnnnnnnnnnnnnnnnnnnnn

SEQ ID NO: 37 - DNA, 153 bp, Artificial Sequence
  LssgRNA_ scaffold
  variation (119)..(153): Nucleotides 119-153 can either all be missing or up to 17 n′s can be missing
  misc_feature (119)..(153): n is a, c, g, or t
ggcaaagaatactgtgcgtgtgctaaggatggaaaaaatccattcaaccacaggattacattatttatctaatcacttaaatctttaagtgattagatgaattaaatgtgattagcacnnnnnnnnnnnnnnnnnnnnnnnnnnnnnnnnnnn

SEQ ID NO: 38 - DNA, 161 bp, Artificial Sequence
  SbsgRNA_ scaffold
  variation (127)..(161): Nucleotides 127-161 can either all be missing or up to 17 n′s can be missing
  misc_feature (127)..(161): n is a, c, g, or t
gtcttagggtatatcccaaatttgtcttagtatgtgcattgcttacagcgacaactaaggtttgtttatcttttttttacattgtaagatgttttacattataaaaagaagataatcttattgcacnnnnnnnnnnnnnnnnnnnnnnnnnnnnnnnnnnn

SEQ ID NO: 39 - DNA, 121 bp, Artificial Sequence
  artsgRA1 scaffold
  variation (87)..(121): Nucleotides 87-121 can either all be missing or up to 17 n′s can be missing
  misc_feature (87)..(121): n is a, c, g, or t
ggtctaaaggacagaatttttcaacgggtgtgccaatggccactttccaggtggcaaagcccgttgaacttcaagcgaagtggcacnnnnnnnnnnnnnnnnnnnnnnnnnnnnnnnnnnn

SEQ ID NO: 40 - DNA, 119 bp, Artificial Sequence
  artsgRA2 scaffold
  variation (85)..(119): Nucleotides 85-119 can either all be missing or up to 17 n′s can be missing
  misc_feature (85)..(119): n is a, c, g, or t
ggtctaaaggacagaagacaacgggaagtgccaatgtgctctttccaagagcaaacaccccgttgacttcaagcgaagtggcacnnnnnnnnnnnnnnnnnnnnnnnnnnnnnnnnnnn

SEQ ID NO: 41 - DNA, 114 bp, Artificial Sequence
  artsgRA3 scaffold
  variation (80)..(114): Nucleotides 80-114 can either all be missing or up to 17 n′s can be missing
  misc_feature (80)..(114): n is a, c, g, or t
ggtctaaaggacagaaaatctgtgcgtgtgccataagtaattaaaaattacccaccacagacttcaagcgaagtggcacnnnnnnnnnnnnnnnnnnnnnnnnnnnnnnnnnnn

SEQ ID NO: 42 - DNA, 126 bp, Artificial Sequence
  artsgRA4 scaffold
  variation (92)..(126): Nucleotides 92-126 can either all be missing or up to 17 n′s can be missing
  misc_feature (92)..(126): n is a, c, g, or t
ggtcgtctataggacggcgagtttttcaacgggtgtgccaatggccactttccaggtggcaaagcccgttgaacttcaagcgaagtggcacnnnnnnnnnnnnnnnnnnnnnnnnnnnnnnnnnnn

SEQ ID NO: 43 - DNA, 124 bp, Artificial Sequence
  artsgRA5 scaffold
  variation (90)..(124): Nucleotides 90-124 can either all be missing or up to 17 n′s can be missing
  misc_feature (90)..(124): n is a, c, g, or t
ggtcgtctataggacggcgaggacaacgggaagtgccaatgtgctctttccaagagcaaacaccccgttgacttcaagcgaagtggcacnnnnnnnnnnnnnnnnnnnnnnnnnnnnnnnnnnn

SEQ ID NO: 44 - DNA, 119 bp, Artificial Sequence
  artsgRA6 scaffold
  variation (85)..(119): Nucleotides 85-119 can either all be missing or up to 17 n′s can be missing
  misc_feature (85)..(119): n is a, c, g, or t
ggtcgtctataggacggcgagaatctgtgcgtgtgccataagtaattaaaaattacccaccacagacttcaagcgaagtggcacnnnnnnnnnnnnnnnnnnnnnnnnnnnnnnnnnnn

SEQ ID NO: 45 - DNA, 125 bp, Artificial Sequence
  artsgRA7 scaffold
  variation (91)..(125): Nucleotides 91-125 can either all be missing or up to 17 n′s can be missing
  misc_feature (91)..(125): n is a, c, g, or t
ggtgacctatagggtcaatgtttttcaacgggtgtgccaatggccactttccaggtggcaaagcccgttgaacttcaagcgaagtggcacnnnnnnnnnnnnnnnnnnnnnnnnnnnnnnnnnnn

SEQ ID NO: 46 - DNA, 123 bp, Artificial Sequence
  artsgRA8 scaffold
  variation (89)..(123): Nucleotides 89-123 can either all be missing or up to 17 n′s can be missing
  misc_feature (89)..(123): n is a, c, g, or t
ggtgacctatagggtcaatggacaacgggaagtgccaatgtgctctttccaagagcaaacaccccgttgacttcaagcgaagtggcacnnnnnnnnnnnnnnnnnnnnnnnnnnnnnnnnnnn

SEQ ID NO: 47 - DNA, 118 bp, Artificial Sequence
  artsgRA9 scaffold
  variation (84)..(118): Nucleotides 84-118 can either all be missing or up to 17 n′s can be missing
  misc_feature (84)..(118): n is a, c, g, or t
ggtgacctatagggtcaatgaatctgtgcgtgtgccataagtaattaaaaattacccaccacagacttcaagcgaagtggcacnnnnnnnnnnnnnnnnnnnnnnnnnnnnnnnnnnn

SEQ ID NO: 48 - DNA, 120 bp, Artificial Sequence
  artsgRA10 scaffold
  variation (86)..(120): Nucleotides 86-120 can either all be missing or up to 17 n′s can be missing
  misc_feature (86)..(120): n is a, c, g, or t
ggtctaaaggacagaatttttcaacgggtgtgccaatggccactttccaggtggcaaagcccgttgagcttcaaagaagtggcacnnnnnnnnnnnnnnnnnnnnnnnnnnnnnnnnnnn

SEQ ID NO: 49 - DNA, 118 bp, Artificial Sequence
  artsgRA11 scaffold
  variation (84)..(118): Nucleotides 84-118 can either all be missing or up to 17 n′s can be missing
  misc_feature (84)..(118): n is a, c, g, or t
ggtctaaaggacagaagacaacgggaagtgccaatgtgctctttccaagagcaaacaccccgttggcttcaaagaagtggcacnnnnnnnnnnnnnnnnnnnnnnnnnnnnnnnnnnn

SEQ ID NO: 50 - DNA, 113 bp, Artificial Sequence
  artsgRA12 scaffold
  variation (79)..(113): Nucleotides 79-113 can either all be missing or up to 17 n′s can be missing
  misc_feature (79)..(113): n is a, c, g, or t
ggtctaaaggacagaaaatctgtgcgtgtgccataagtaattaaaaattacccaccacaggcttcaaagaagtggcacnnnnnnnnnnnnnnnnnnnnnnnnnnnnnnnnnnn

SEQ ID NO: 51 - DNA, 125 bp, Artificial Sequence
  artsgRA13 scaffold
  variation (91)..(125): Nucleotides 91-125 can either all be missing or up to 17 n′s can be missing
  misc_feature (91)..(125): n is a, c, g, or t
ggtcgtctataggacggcgagtttttcaacgggtgtgccaatggccactttccaggtggcaaagcccgttgagcttcaaagaagtggcacnnnnnnnnnnnnnnnnnnnnnnnnnnnnnnnnnnn

SEQ ID NO: 52 - DNA, 123 bp, Artificial Sequence
  artsgRA14 scaffold
  variation (89)..(123): Nucleotides 89-123 can either all be missing or up to 17 n′s can be missing
  misc_feature (89)..(123): n is a, c, g, or t
ggtcgtctataggacggcgaggacaacgggaagtgccaatgtgctctttccaagagcaaacaccccgttggcttcaaagaagtggcacnnnnnnnnnnnnnnnnnnnnnnnnnnnnnnnnnnn

SEQ ID NO: 53 - DNA, 118 bp, Artificial Sequence
  artsgRA15 scaffold
  variation (84)..(118): Nucleotides 84-118 can either all be missing or up to 17 n′s can be missing
  misc_feature (84)..(118): n is a, c, g, or t
ggtcgtctataggacggcgagaatctgtgcgtgtgccataagtaattaaaaattacccaccacaggcttcaaagaagtggcacnnnnnnnnnnnnnnnnnnnnnnnnnnnnnnnnnnn

SEQ ID NO: 54 - DNA, 124 bp, Artificial Sequence
  artsgRA16 scaffold
  variation (90)..(124): Nucleotides 90-124 can either all be missing or up to 17 n′s can be missing
  misc_feature (90)..(124): n is a, c, g, or t
ggtgacctatagggtcaatgtttttcaacgggtgtgccaatggccactttccaggtggcaaagcccgttgagcttcaaagaagtggcacnnnnnnnnnnnnnnnnnnnnnnnnnnnnnnnnnnn

SEQ ID NO: 55 - DNA, 122 bp, Artificial Sequence
  artsgRA17 scaffold
  variation (88)..(122): Nucleotides 88-122 can either all be missing or up to 17 n′s can be missing
  misc_feature (88)..(122): n is a, c, g, or t
ggtgacctatagggtcaatggacaacgggaagtgccaatgtgctctttccaagagcaaacaccccgttggcttcaaagaagtggcacnnnnnnnnnnnnnnnnnnnnnnnnnnnnnnnnnnn

SEQ ID NO: 56 - DNA, 117 bp, Artificial Sequence
  artsgRA18 scaffold
  variation (83)..(117): Nucleotides 83-117 can either all be missing or up to 17 n′s can be missing
  misc_feature (83)..(117): n is a, c, g, or t
ggtgacctatagggtcaatgaatctgtgcgtgtgccataagtaattaaaaattacccaccacaggcttcaaagaagtggcacnnnnnnnnnnnnnnnnnnnnnnnnnnnnnnnnnnn

SEQ ID NO: 57 - DNA, 124 bp, Artificial Sequence
  artsgRA19 scaffold
  variation (90)..(124): Nucleotides 90-124 can either all be missing or up to 17 n′s can be missing
  misc_feature (90)..(124): n is a, c, g, or t
ggtctaaaggacagaatttttcaacgggtgtgccaatggccactttccaggtggcaaagcccgttgagattatctatgatgattggcacnnnnnnnnnnnnnnnnnnnnnnnnnnnnnnnnnnn

SEQ ID NO: 58 - DNA, 122 bp, Artificial Sequence
  artsgRA20 scaffold
  variation (88)..(122): Nucleotides 88-122 can either all be missing or up to 17 n′s can be missing
  misc_feature (88)..(122): n is a, c, g, or t
ggtctaaaggacagaagacaacgggaagtgccaatgtgctctttccaagagcaaacaccccgttggattatctatgatgattggcacnnnnnnnnnnnnnnnnnnnnnnnnnnnnnnnnnnn

SEQ ID NO: 59 - DNA, 117 bp, Artificial Sequence
  artsgRA21 scaffold
  variation (83)..(117): Nucleotides 83-117 can either all be missing or up to 17 n′s can be missing
  misc_feature (83)..(117): n is a, c, g, or t
ggtctaaaggacagaaaatctgtgcgtgtgccataagtaattaaaaattacccaccacaggattatctatgatgattggcacnnnnnnnnnnnnnnnnnnnnnnnnnnnnnnnnnnn

SEQ ID NO: 60 - DNA, 129 bp, Artificial Sequence
  artsgRA22 scaffold
  variation (95)..(129): Nucleotides 95-129 can either all be missing or up to 17 n′s can be missing
  misc_feature (95)..(129): n is a, c, g, or t
ggtcgtctataggacggcgagtttttcaacgggtgtgccaatggccactttccaggtggcaaagcccgttgagattatctatgatgattggcacnnnnnnnnnnnnnnnnnnnnnnnnnnnnnnnnnnn

SEQ ID NO: 61 - DNA, 127 bp, Artificial Sequence
  artsgRA23 scaffold
  variation (93)..(127): Nucleotides 93-127 can either all be missing or up to 17 n′s can be missing
  misc_feature (93)..(127): n is a, c, g, or t
ggtcgtctataggacggcgaggacaacgggaagtgccaatgtgctctttccaagagcaaacaccccgttggattatctatgatgattggcacnnnnnnnnnnnnnnnnnnnnnnnnnnnnnnnnnnn

SEQ ID NO: 62 - DNA, 122 bp, Artificial Sequence
  artsgRA24 scaffold
  variation (88)..(122): Nucleotides 88-122 can either all be missing or up to 17 n′s can be missing
  misc_feature (88)..(122): n is a, c, g, or t
ggtcgtctataggacggcgagaatctgtgcgtgtgccataagtaattaaaaattacccaccacaggattatctatgatgattggcacnnnnnnnnnnnnnnnnnnnnnnnnnnnnnnnnnnn

SEQ ID NO: 63 - DNA, 128 bp, Artificial Sequence
  artsgRA25 scaffold
  variation (94)..(128): Nucleotides 94-128 can either all be missing or up to 17 n′s can be missing
  misc_feature (94)..(128): n is a, c, g, or t
ggtgacctatagggtcaatgtttttcaacgggtgtgccaatggccactttccaggtggcaaagcccgttgagattatctatgatgattggcacnnnnnnnnnnnnnnnnnnnnnnnnnnnnnnnnnnn

SEQ ID NO: 64 - DNA, 126 bp, Artificial Sequence
  artsgRA26 scaffold
  variation (92)..(126): Nucleotides 92-126 can either all be missing or up to 17 n′s can be missing
  misc_feature (92)..(126): n is a, c, g, or t
ggtgacctatagggtcaatggacaacgggaagtgccaatgtgctctttccaagagcaaacaccccgttggattatctatgatgattggcacnnnnnnnnnnnnnnnnnnnnnnnnnnnnnnnnnnn

SEQ ID NO: 65 - DNA, 121 bp, Artificial Sequence
  artsgRA27 scaffold
  variation (87)..(121): Nucleotides 87-121 can either all be missing or up to 17 n′s can be missing
  misc_feature (87)..(121): n is a, c, g, or t
ggtgacctatagggtcaatgaatctgtgcgtgtgccataagtaattaaaaattacccaccacaggattatctatgatgattggcacnnnnnnnnnnnnnnnnnnnnnnnnnnnnnnnnnnn

SEQ ID NO: 66 - DNA, 117 bp, Artificial Sequence
  artsgRA28 scaffold
  variation (83)..(117): Nucleotides 83-117 can either all be missing or up to 17 n′s can be missing
  misc_feature (83)..(117): n is a, c, g, or t
ggtctaaaggacagaacaacgggatgtgccaatgcactctttccaggagtgaacaccccgttgacttcaagcgaagtggcacnnnnnnnnnnnnnnnnnnnnnnnnnnnnnnnnnnn

SEQ ID NO: 67 - DNA, 122 bp, Artificial Sequence
  artsgRA29 scaffold
  variation (88)..(122): Nucleotides 88-122 can either all be missing or up to 17 n′s can be missing
  misc_feature (88)..(122): n is a, c, g, or t
ggtcgtctataggacggcgagcaacgggatgtgccaatgcactctttccaggagtgaacaccccgttgacttcaagcgaagtggcacnnnnnnnnnnnnnnnnnnnnnnnnnnnnnnnnnnn

SEQ ID NO: 68 - DNA, 134 bp, Artificial Sequence
  artsgRA30 scaffold
  variation (100)..(134): Nucleotides 100-134 can either all be missing or up to 17 n′s can be missing
  misc_feature (100)..(134): n is a, c, g, or t
ggaattgccgatctataggacggcagatttttttcaacgggtgtgccaatggccactttccaggtggcaaagcccgttgaacttcaagcgaagtggcacnnnnnnnnnnnnnnnnnnnnnnnnnnnnnnnnnnn

SEQ ID NO: 69 - DNA, 132 bp, Artificial Sequence
  artsgRA31 scaffold
  variation (98)..(132): Nucleotides 98-132 can either all be missing or up to 17 n′s can be missing
  misc_feature (98)..(132): n is a, c, g, or t
ggaattgccgatctataggacggcagattgacaacgggaagtgccaatgtgctctttccaagagcaaacaccccgttgacttcaagcgaagtggcacnnnnnnnnnnnnnnnnnnnnnnnnnnnnnnnnnnn

SEQ ID NO: 70 - DNA, 130 bp, Artificial Sequence
  artsgRA32 scaffold
  variation (96)..(130): Nucleotides 96-130 can either all be missing or up to 17 n′s can be missing
  misc_feature (96)..(130): n is a, c, g, or t
ggaattgccgatctataggacggcagattcaacgggatgtgccaatgcactctttccaggagtgaacaccccgttgacttcaagcgaagtggcacnnnnnnnnnnnnnnnnnnnnnnnnnnnnnnnnnnn

SEQ ID NO: 71 - DNA, 116 bp, Artificial Sequence
  artsgRA33 scaffold
  variation (82)..(116): Nucleotides 82-116 can either all be missing or up to 17 n′s can be missing
  misc_feature (82)..(116): n is a, c, g, or t
ggtctaaaggacagaacaacgggatgtgccaatgcactctttccaggagtgaacaccccgttggcttcaaagaagtggcacnnnnnnnnnnnnnnnnnnnnnnnnnnnnnnnnnnn

SEQ ID NO: 72 - DNA, 121 bp, Artificial Sequence
  artsgRA34 scaffold
  variation (87)..(121): Nucleotides 87-121 can either all be missing or up to 17 n′s can be missing
  misc_feature (87)..(121): n is a, c, g, or t
ggtcgtctataggacggcgagcaacgggatgtgccaatgcactctttccaggagtgaacaccccgttggcttcaaagaagtggcacnnnnnnnnnnnnnnnnnnnnnnnnnnnnnnnnnnn

SEQ ID NO: 73 - DNA, 133 bp, Artificial Sequence
  artsgRA35 scaffold
  variation (99)..(133): Nucleotides 99-133 can either all be missing or up to 17 n′s can be missing
  misc_feature (99)..(133): n is a, c, g, or t
ggaattgccgatctataggacggcagatttttttcaacgggtgtgccaatggccactttccaggtggcaaagcccgttgagcttcaaagaagtggcacnnnnnnnnnnnnnnnnnnnnnnnnnnnnnnnnnnn

SEQ ID NO: 74 - DNA, 131 bp, Artificial Sequence
  artsgRA36 scaffold
  variation (97)..(131): Nucleotides 97-131 can either all be missing or up to 17 n′s can be missing
  misc_feature (97)..(131): n is a, c, g, or t
ggaattgccgatctataggacggcagattgacaacgggaagtgccaatgtgctctttccaagagcaaacaccccgttggcttcaaagaagtggcacnnnnnnnnnnnnnnnnnnnnnnnnnnnnnnnnnnn

SEQ ID NO: 75 - DNA, 129 bp, Artificial Sequence
  artsgRA37 scaffold
  variation (95)..(129): Nucleotides 95-129 can either all be missing or up to 17 n′s can be missing
  misc_feature (95)..(129): n is a, c, g, or t
ggaattgccgatctataggacggcagattcaacgggatgtgccaatgcactctttccaggagtgaacaccccgttggcttcaaagaagtggcacnnnnnnnnnnnnnnnnnnnnnnnnnnnnnnnnnnn

SEQ ID NO: 76 - DNA, 20 bp, Artificial Sequence
  CCR5 site 10
tccttctcctgaacaccttc

SEQ ID NO: 77 - DNA, 20 bp, Artificial Sequence
  CCR5 site 28
tttggcctgaataattgcag

SEQ ID NO: 78 - DNA, 20 bp, Artificial Sequence
  DNMT1 site 16
cccttcagctaaaataaagg

SEQ ID NO: 79 - DNA, 20 bp, Artificial Sequence
  RNF2 site 8
tagtcatggtgttcttcaac

SEQ ID NO: 80 - DNA, 20 bp, Artificial Sequence
  Site 5
gctctcaagacccacaatcc

SEQ ID NO: 81 - protein, 5 aa, Artificial Sequence
  Artificially synthesized peptide sequence
KKRKV

SEQ ID NO: 82 - protein, 7 aa, Artificial Sequence
  Artificially synthesized peptide sequence
PKKKRKV

SEQ ID NO: 83 - protein, 11 aa, Artificial Sequence
  Artificially synthesized peptide sequence
SGGSPKKKRKV